(19) United States
(12) Patent Application Publication
(10) Pub. No.: US 2025/0041214 A1
(43) Pub. Date: Feb. 6, 2025

(54) INHALABLE OR INGESTIBLE LACTIC ACID COMPOSITIONS FOR THE TREATMENT OF CHRONIC LUNG DISEASE

(71) Applicant: Alveolus Bio, Inc., Birmingham, AL (US)

(72) Inventors: Arthur Tipton, Birmingham, AL (US); Andrew O'Connor, Birmingham, AL (US)

(21) Appl. No.: 18/717,210

(22) PCT Filed: Dec. 15, 2022
(86) PCT No.: PCT/US2022/052972

§ 371 Date: Jun. 6, 2024

Related U.S. Application Data
(60) Provisional application No. 63/290,297, filed on Dec. 16, 2021.

(51) Int. Cl. A61K 9/00; A61K 9/16; A61K 31/19; A61K 31/765; A61K 45/06; A61P 31/04

(52) U.S. Cl. CPC A61K 9/0075; A61K 9/1623; A61K 31/19; A61K 31/765; A61K 45/06; A61P 31/04

(57) ABSTRACT

The technology described herein is directed to pharmaceutical compositions comprising a lactic acid-producing compound, which is formulated for administration by inhalation and/or oral administration. Also described herein are unit dosage forms of such pharmaceutical compositions, devices comprising such pharmaceutical compositions, methods of producing such pharmaceutical compositions, and methods of treating bronchopulmonary diseases, using such pharmaceutical compositions.

### CROSS-REFERENCE TO RELATED APPLICATIONS

This application claims benefit under 35 U.S.C. § 119(e) of U.S. Provisional Application No. 63/290,297 filed Dec. 16, 2021, the contents of which are incorporated herein by reference in their entirety.

### TECHNICAL FIELD

The technology described herein relates to lactic acid compositions for the treatment of lung disease.

### BACKGROUND

Chronic inflammatory lung disease is associated with dysbiosis of the lung microbiome. The term “dysbiosis” as used here is used to refer to an imbalance between commensal and pathogenic bacteria. One way to address dysbiosis is to increase the population of commensal bacteria in the microenvironment. Lactobacillus is a family of lactic acid-producing commensals that are known for their anti-inflammatory properties. Lactobacillus acidophilus counteracts E. coli mediated inhibition of butyrate uptake by intestinal epithelial cells. Lacticaseibacillus rhamnosus (previously Lactobacillus rhamnosus; e.g., strain LGG) decreases likelihood of nosocomial respiratory and gastrointestinal infection in the pediatric population. Lactiplantibacillus plantarum (previously Lactobacillus plantarum, e.g., strain 299v) protects against antibiotic mediated short chain fatty acid (SCFA) depletion. A probiotic blend including L. acidophilus, rhamnosus, and plantarum showed a decrease in pro-inflammatory and increase in anti-inflammatory factors in healthy samples. See e.g., Enaud et al. 2020, Front Cell Infect Microbiol 10:9; Saint-Criq et al. 2021, Ageing Res Rev 66:101235; Kumar et al. 2015, Am J Physiol Gastrointest Liver Physiol 309 (7): G602-607; Hojsak et al. 2010, Pediatrics 125 (5): e1171-1177; Wullt et al. 2007, Digestive Diseases and Sciences 52 (9):2082; Moens et al. 2019, International Journal of Pharmaceutics 555:1-10; the contents of each of which are incorporated herein by reference in their entireties.

Studies have aimed to isolate the active extract or metabolite that gives Lactobacillus its anti-inflammatory capacity. Administration of a Lactobacillus blend led to a reduction of neutrophilic inflammatory marker Matrix Metalloproteinase-9 (MMP-9) in dysbiosis models of lung injury in vitro and in vivo. Elevated levels of MMP-9 are associated with chronic inflammatory lung diseases and non-small cell lung cancer (NSCLC). See e.g., International Patent Application WO 2018/191073; Atkinson and Senior 2003, Am J Respir Cell Mol Biol 28 (1):12-24; El-Badrawy et al. 2014, J Bronchology Interv Pulmonol 21 (4):327-334; the contents of each of which are incorporated herein by reference in their entireties.

There is a need for therapeutics comprising anti-inflammatory extracts or metabolites from bacteria such as Lactobacillus, especially as it relates to chronic inflammatory lung disease.

### SUMMARY

Lactic acid has been identified herein as an anti-inflammatory compound produced by microbiota. Accordingly, the technology described herein is directed to pharmaceutical compositions comprising a lactic acid-producing compound, which is formulated for administration by inhalation and/or oral administration. Also described herein are unit dosage forms of such pharmaceutical compositions, devices comprising such pharmaceutical compositions, methods of producing such pharmaceutical compositions, and methods of treating bronchopulmonary diseases, among others, using such pharmaceutical compositions.

Specifically, the present disclosure encompasses embodiments of an inhaled or ingested lactic acid product that delivers a matrix of constituents to the lungs or gut to reduce neutrophilic inflammation characteristic of many chronic lung diseases. Administration of lactic acid led to MMP-9 reduction in E. coli-treated human bronchial epithelial cells (HBE) (see e.g., FIG. 2). The present disclosure thus encompasses embodiments of this inhaled or ingested lactic acid-related product—either in crystalline solid form, amorphous solid form, or a mixture—delivering lactic acid or similar acids.

In one aspect, described herein is a pharmaceutical composition comprising: (a) a lactic acid-producing compound selected from: (i) a polymeric compound that can produce lactic acid; (ii) a non-polymeric compound that can produce lactic acid; or (iii) lactic acid; and (b) a pharmaceutically acceptable excipient, stabilizer, or additive; wherein the composition is formulated for administration to the lungs.

In some embodiments of any of the aspects, the composition is formulated for administration by inhalation.

In some embodiments of any of the aspects, the lactic acid-producing compound produces lactic acid upon delivery to a target tissue.

In some embodiments of any of the aspects, the target tissue is a target bronchopulmonary tissue.

In some embodiments of any of the aspects, the target bronchopulmonary tissue is the lungs, the trachea, the bronchi, the bronchioles, and/or the alveoli.

In some embodiments of any of the aspects, the target tissue is a distal tissue site from the lungs delivered via the cardiovascular system or lymphatic system.

In some embodiments of any of the aspects, the lactic acid-producing compound comprises a D enantiomer of lactic acid, an L enantiomer of lactic acid, or a racemic mixture of D and L enantiomers of lactic acid.

In some embodiments of any of the aspects, the non-polymeric lactic acid-producing compound can be metabolized in the target tissue to produce lactic acid.

In some embodiments of any of the aspects, the non-polymeric lactic acid-producing compound is an inorganic salt of lactic acid, an ester of lactic acid, or lactide.

In some embodiments of any of the aspects, the inorganic salt of lactic acid is sodium lactate, potassium lactate, calcium lactate, or magnesium lactate.

In some embodiments of any of the aspects, the ester of lactic acid is ethyl lactate, propyl lactate, butyl lactate, pentyl lactate, hexyl lactate, heptyl lactate, octyl lactate, nonyl lactate, decyl lactate, undecyl lactate, or dodecyl lactate.

In some embodiments of any of the aspects, the polymeric lactic acid-producing compound can be hydrolyzed in the target tissue to produce lactic acid.

In some embodiments of any of the aspects, the polymeric lactic acid-producing compound is a polylactic acid (PLA).

In some embodiments of any of the aspects, the polylactic acid is poly(L-lactide) (PLLA), poly(D,L-lactide) (PDLLA), or poly(D-lactide) (PDLA).

In some embodiments of any of the aspects, the polylactic acid is poly(D,L-lactide) (PDLLA).

In some embodiments of any of the aspects, the polymeric lactic acid-producing compound is poly(lactic-co-glycolic acid) (PLGA).

In some embodiments of any of the aspects, the polymeric lactic acid-producing compound is poly(lactic acid-co-caprolactone).

In some embodiments of any of the aspects, the composition comprises at least 1.0% lactic acid-producing compound by weight.

In some embodiments of any of the aspects, the composition comprises at least one excipient or at least one stabilizer.

In some embodiments of any of the aspects, the pharmaceutical composition further comprises at least one excipient.

In some embodiments of any of the aspects, the pharmaceutical composition further comprises at least two excipients.

In some embodiments of any of the aspects, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments of any of the aspects, the excipient is leucine and/or trehalose.

In some embodiments of any of the aspects, the composition comprises at least 5.0% excipient by weight.

In some embodiments of any of the aspects, the composition comprises at least 5.0% of a first excipient by weight, and at least 5.0% of a second excipient by weight.

In some embodiments of any of the aspects, the pharmaceutical composition further comprises at least one stabilizer.

In some embodiments of any of the aspects, the stabilizer comprises a surfactant.

In some embodiments of any of the aspects, the stabilizer is selected from the group consisting of: mannitol, carboxymethyl cellulose (CMC), polyvinyl alcohol (PVA), polysorbate, and poloxamer.

In some embodiments of any of the aspects, the stabilizer is a polysorbate; poloxamer; or polyvinyl alcohol.

In some embodiments of any of the aspects, the stabilizer is Polysorbate 20, Polysorbate 40, Polysorbate 60, or Polysorbate 80.

In some embodiments of any of the aspects, the stabilizer is Polysorbate 80.

In some embodiments of any of the aspects, the stabilizer is Poloxamer 184, Poloxamer 185, Poloxamer 188, Poloxamer 234, Poloxamer 235, Poloxamer 238, Poloxamer 333, Poloxamer 334, Poloxamer 335, Poloxamer 338, Poloxamer 403, or Poloxamer 407.

In some embodiments of any of the aspects, the stabilizer is poloxamer 188.

In some embodiments of any of the aspects, the composition comprises at least 0.10% stabilizer by weight.

In some embodiments of any of the aspects, the composition comprises at least one excipient and at least one stabilizer.

In some embodiments of any of the aspects, the pharmaceutical composition further comprises at least one of the following: (a) a pore-forming agent; (b) an adhesion agent; (c) a pH-modulating agent; and/or (d) an ester-hydrolysis-inducing agent.

In some embodiments of any of the aspects, the pore-forming agent is selected from the group consisting of: NaCl, sucrose, polyethylene glycol (PEG), and polyvinylpyrrolidone (PVP).

In some embodiments of any of the aspects, the adhesion agent is selected from the group consisting of: sugars, adhesive polymers, and amine-containing compounds.

In some embodiments of any of the aspects, the pH-modulating agent is a buffer, an acid, or a base.

In some embodiments of any of the aspects, the ester-hydrolysis-inducing agent comprises an amine.

In some embodiments of any of the aspects, the pharmaceutical composition further comprises at least one acid-generating molecule that can generate acid, wherein the at least one acid-generating molecule is not lactic acid or does not comprise lactic acid.

In some embodiments of any of the aspects, the acid-generating molecule is selected from the group consisting of: acetic acid, a hydroxy acid, a multiple functional acid, and an aromatic acid, and esters, salts, and polymers thereof.

In some embodiments of any of the aspects, the acid-generating molecule is selected from the group consisting of: acetic acid, glycolic acid, citric acid, and salicylic acid, and esters, salts, and polymers thereof.

In some embodiments of any of the aspects, the acid-generating molecule is acetylsalicylic acid.

In some embodiments of any of the aspects, the composition comprises at least one additional therapeutic for a chronic bronchopulmonary disorder.

In some embodiments of any of the aspects, the at least one additional therapeutic is microencapsulated.

In some embodiments of any of the aspects, the at least one additional therapeutic is covalently linked with a degradable linker to the lactic acid-producing compound.

In some embodiments of any of the aspects, the pharmaceutical composition is co-administered with at least one additional therapeutic for a chronic or infectious bronchopulmonary disorder.

In some embodiments of any of the aspects, the at least one additional therapeutic is an anti-inflammatory, an antimicrobial, an antiviral, an antifungal, a vasodilator, or a bronchodilator.

In some embodiments of any of the aspects, the anti-inflammatory is selected from the group consisting of: non-steroidal anti-inflammatory drugs (NSAIDs); corticosteroids; glucocorticoids; methotrexate; sulfasalazine; leflunomide; anti-tumor necrosis factor (TNF) medications; cyclophosphamide; pro-resolving lipid mediators; mycophenolate; opiates; and barbiturates.

In some embodiments of any of the aspects, the antimicrobial is selected from the group consisting of: aminoglycosides; ansamycins; beta-lactams; bis-biguanides; carbacephems; carbapenems; cationic polypeptides; cephalosporins; fluoroquinolones; glycopeptides; iron-sequestering glycoproteins; linosamides; lipopeptides; macrolides; monobactams; nitrofurans; oxazolidinones; penicillins; polypeptides; quaternary ammonium compounds; quinolones; silver compounds; sulfonamides; and tetracyclines.

In some embodiments of any of the aspects, the vasodilator is selected from the group consisting of: an angiotensin converting enzyme (ACE) inhibitor; an angiotensin receptor blocker (ARB); a calcium channel blocker (CCB); and a nitric-oxide-producing compound.

In some embodiments of any of the aspects, the bronchodilator is selected from the group consisting of: albuterol, levalbuterol, epinephrine, salmeterol, formoterol, ipratropium bromide, tiotropium bromide, theophylline, and aminophylline.

In some embodiments of any of the aspects, the composition is formulated as a bolus dose.

In some embodiments of any of the aspects, the pharmaceutical composition comprises: (a) the polymeric lactic acid-producing compound and/or the non-polymeric lactic acid-producing compound; and (b) a bolus dose of lactic acid.

In some embodiments of any of the aspects, the composition is formulated as microspheres.

In some embodiments of any of the aspects, the microspheres have a diameter of at least 1 μm to at most 1 mm.

In some embodiments of any of the aspects, the composition comprises a plurality of dried particles.

In some embodiments of any of the aspects, the dried particles have a Dv50 of at least 0.5 μm.

In some embodiments of any of the aspects, the dried particles have a median mass aerodynamic diameter (MMAD) of at least 1.5 μm to at most 7.5 μm.

In some embodiments of any of the aspects, the dried particles have a median mass aerodynamic diameter (MMAD) of at most 5.0 μm.

In some embodiments of any of the aspects, the dried particles have a dispersibility of less than 2.0.

In some embodiments of any of the aspects, the dried particles have a dispersibility of at least 0.5 to 1.0.

In some embodiments of any of the aspects, the dried particles have a dispersibility of at least 0.9.

In some embodiments of any of the aspects, the dried particles have a delivered dose of at least 25.0% to at most 125% of the composition by mass to a target tissue.

In some embodiments of any of the aspects, the dried particles have a delivered dose of at least 30% of the lactic acid-producing compound by mass to a target tissue.

In some embodiments of any of the aspects, the dried particles have a delivered dose of at least 7.8 mg per unit dose of the lactic acid-producing compound by mass to a target tissue.

In some embodiments of any of the aspects, the dried particles have a delivered dose of at most 50 mg per unit dose of the lactic acid-producing compound by mass to a target tissue.

In some embodiments of any of the aspects, the dried particles have a bulk density of at least 0.1 g/cm³ to 0.8 g/cm³.

In some embodiments of any of the aspects, the dried particles have a bulk density of at least 0.5 g/cm³.

In some embodiments of any of the aspects, the dried particles have a tapped density of at least 0.2 g/cm³ to 1.0 g/cm³.

In some embodiments of any of the aspects, the dried particles have a tapped density of at least 0.6 g/cm³.

In some embodiments of any of the aspects, the dried particles have a moisture content of at least 1.0% to 7.0% water by weight.

In some embodiments of any of the aspects, the dried particles have a moisture content of at least 2.3% water by weight.

In some embodiments of any of the aspects, the composition is formulated for delivery to the trachea, the bronchi, the bronchioles, and/or the alveoli.

In some embodiments of any of the aspects, the composition is formulated for delivery to the lungs.

In some embodiments of any of the aspects, the composition is formulated as a capsule or a tablet.

In some embodiments of any of the aspects, the composition comprises at least 7.8 mg of the lactic acid-producing compound per unit dose that is deliverable to a target tissue.

In some embodiments of any of the aspects, the composition comprises at least 15 mg of the lactic acid-producing compound per unit dose.

In some embodiments of any of the aspects, the composition is formulated for delivery by an inhaler.

In some embodiments of any of the aspects, the composition is formulated for delivery by a dry powder inhaler (DPI).

In some embodiments of any of the aspects, the composition is formulated for delivery by a metered dose inhaler (MDI).

In some embodiments of any of the aspects, the composition is formulated for delivery by a soft mist inhaler (SMI).

In some embodiments of any of the aspects, the pharmaceutical composition is in combination with an inhaler.

In one aspect, described herein is an inhalation device for bronchopulmonary delivery comprising: (a) an inhaler; and (b) a container containing a pharmaceutical composition as described herein.

In some embodiments of any of the aspects, the inhaler is a dry powder inhaler (DPI).

In some embodiments of any of the aspects, the inhaler is a metered dose inhaler (MDI).

In some embodiments of any of the aspects, the inhaler is a soft mist inhaler (SMI).

In some embodiments of any of the aspects, the inhaler comprises: (a) a mouthpiece comprising an opening; and (b) means for aerosolizing or dispersing the pharmaceutical composition in the container.

In one aspect, described herein is a method of preparing a spray-dried pharmaceutical composition comprising a lactic acid-producing compound, comprising: (a) preparing a liquid feedstock comprising the lactic acid-producing compound; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the lactic acid-producing compound.

In one aspect, described herein is a method of preparing a spray-dried pharmaceutical composition comprising a lactic acid-producing compound, comprising: (a) obtaining a liquid feedstock comprising the lactic acid-producing compound; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the lactic acid-producing compound.

In some embodiments of any of the aspects, the step of preparing the liquid feedstock comprises dissolving a solid feedstock into an aqueous solution.

In some embodiments of any of the aspects, the step of preparing the liquid feedstock comprises dissolving a solid feedstock into an organic solution.

In some embodiments of any of the aspects, the step of preparing the liquid feedstock comprises: (a) dissolving a lactic acid-producing compound into an organic solution; (b) dissolving a solid feedstock into an aqueous solution; and (c) combining the resultant solutions from (a) and (b) to produce a liquid feedstock.

In some embodiments of any of the aspects, the solid feedstock comprises: (a) at least 50% lactic acid-producing compound by weight; (b) at least 10% excipient by weight; and/or (c) at least 1% stabilizer by weight.

In some embodiments of any of the aspects, the solid feedstock comprises: (a) at least 50% lactic acid-producing compound by weight; (b) at least 5% of a first excipient by weight; (c) at least 5% of a second excipient by weight; and/or (d) at least 1% stabilizer by weight.

In some embodiments of any of the aspects, the solid feedstock comprises at least 20% to at most 80% lactic acid-producing compound by weight.

In some embodiments of any of the aspects, the solid feedstock comprises at least 1% to at most 15% excipient by weight.

In some embodiments of any of the aspects, the solid feedstock comprises at least 2.5%-7.5% of a first excipient by weight, and at least 2.5%-7.5% of a second excipient by weight.

In some embodiments of any of the aspects, the solid feedstock comprises at least 10% to at most 50% stabilizer by weight.

In some embodiments of any of the aspects, the liquid feedstock comprises at least 0.1 g/L solid feedstock dissolved in an aqueous solution.

In some embodiments of any of the aspects, the liquid feedstock comprises at least 5 g/L solid feedstock dissolved in an aqueous solution.

In some embodiments of any of the aspects, the liquid feedstock comprises at least 0.01% to at most 10% solid feedstock dissolved in an aqueous solution.

In some embodiments of any of the aspects, the liquid feedstock comprises at least 0.5% solid feedstock dissolved in an aqueous solution.

In some embodiments of any of the aspects, 0.5 L of the liquid feedstock comprises: (a) at least 2.00 g lactic acid-producing compound; (b) at least 0.80 g excipient; (c) at least 1.2 g stabilizer; (d) at least 31.36 g organic solution; and/or (e) at least 464.64 g aqueous solution.

In some embodiments of any of the aspects, 0.5 L of the liquid feedstock comprises: (a) at least 2.00 g lactic acid-producing compound; (b) at least 0.40 g of a first excipient; (c) at least 0.40 g of a second excipient; (d) at least 1.2 g stabilizer; (e) at least 31.36 g organic solution; and/or (f) at least 464.64 g aqueous solution.

In some embodiments of any of the aspects, 0.5 L of the liquid feedstock comprises at least 0.1 g to at most 10 g lactic acid-producing compound.

In some embodiments of any of the aspects, 0.5 L of the liquid feedstock comprises at least 0.1 g to at most 10 g excipient.

In some embodiments of any of the aspects, 0.5 L of the liquid feedstock comprises at least 0.05 g to at most 5 g of a first excipient, and at least 0.05 g to at most 5 g of a second excipient.

In some embodiments of any of the aspects, 0.5 L of the liquid feedstock comprises at least 0.1 g to at most 10 g stabilizer.

In some embodiments of any of the aspects, 0.5 L of the liquid feedstock comprises at least 10 g to at most 50 g organic solution.

In some embodiments of any of the aspects, 0.5 L of the liquid feedstock comprises at least 420 g to at most 490 g aqueous solution.

In some embodiments of any of the aspects, the liquid feedstock comprises: (a) at least 0.40% lactic acid-producing compound; (b) at least 0.16% excipient; (c) at least 0.24% stabilizer; (d) at least 6.27% organic solution; and/or (e) at least 92.93% aqueous solution.

In some embodiments of any of the aspects, the liquid feedstock comprises: (a) at least 0.40% lactic acid-producing compound by weight; (b) at least 0.08% of a first excipient by weight; (c) at least 0.08% of a second excipient by weight; (d) at least 0.24% stabilizer; (e) at least 6.27% organic solution; and/or (f) at least 92.93% aqueous solution by weight.

In some embodiments of any of the aspects, the liquid feedstock comprises at least 0.01% to at most 1.0% lactic acid-producing compound by weight.

In some embodiments of any of the aspects, the liquid feedstock comprises at least 0.01% to at most 10% excipient by weight.

In some embodiments of any of the aspects, the liquid feedstock comprises at least 0.005% to at most 5% of a first excipient by weight, and at least 0.005% to at most 5% of a second excipient by weight.

In some embodiments of any of the aspects, the liquid feedstock comprises at least 0.01% to at most 1.0% stabilizer by weight.

In some embodiments of any of the aspects, the liquid feedstock comprises at least 1% to at most 5% organic solution by weight.

In some embodiments of any of the aspects, the liquid feedstock comprises at least 90% to at most 99.9% aqueous solution by weight.

In some embodiments of any of the aspects, the lactic acid-producing compound is selected from: (a) a polymeric compound that can produce lactic acid; (b) a non-polymeric compound that can produce lactic acid; or (c) lactic acid.

In some embodiments of any of the aspects, the non-polymeric lactic acid-producing compound is an inorganic salt of lactic acid, an ester of lactic acid, or lactide.

In some embodiments of any of the aspects, the polymeric lactic acid-producing compound is a polylactic acid (PLA).

In some embodiments of any of the aspects, the polylactic acid is poly(D,L-lactide) (PDLLA).

In some embodiments of any of the aspects, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments of any of the aspects, the excipient is leucine and/or trehalose.

In some embodiments of any of the aspects, the stabilizer is a polysorbate; poloxamer; or polyvinyl alcohol.

In some embodiments of any of the aspects, the stabilizer is Poloxamer 188.

In some embodiments of any of the aspects, the organic solution is acetone.

In some embodiments of any of the aspects, the aqueous solution is water.

In some embodiments of any of the aspects, the liquid feedstock further comprises at least one additional therapeutic.

In some embodiments of any of the aspects, the at least one additional therapeutic is selected from the group consisting of: an anti-inflammatory, an antimicrobial, an antiviral, an antifungal, a vasodilator, and a bronchodilator.

In some embodiments of any of the aspects, the atomization nozzle into the drying chamber has a diameter of at least 1.2 mm.

In some embodiments of any of the aspects, the droplets of liquid feedstock produced by the atomization nozzle into the drying chamber have a diameter of at least 1.2 um.

In some embodiments of any of the aspects, the droplets of liquid feedstock have a flow rate through the drying chamber of at least 5 g/min.

In some embodiments of any of the aspects, the droplets of liquid feedstock have a flow rate through the drying chamber of at least 15 g/min.

In some embodiments of any of the aspects, the droplets of liquid feedstock have a flow rate through the drying chamber of at most 1000 g/min.

In some embodiments of any of the aspects, the heated, pressurized gas is heated before being inlet into the drying chamber.

In some embodiments of any of the aspects, the heated, pressurized gas is inlet into the drying chamber at a temperature of at least 100° C.

In some embodiments of any of the aspects, the heated, pressurized gas is inlet into the drying chamber at a temperature of at least 135° C.

In some embodiments of any of the aspects, the heated, pressurized gas is inlet into the drying chamber at a temperature of at most 195° C.

In some embodiments of any of the aspects, the heated, pressurized gas is outlet from the drying chamber at a temperature of at least 40° C.

In some embodiments of any of the aspects, the heated, pressurized gas is outlet from the drying chamber at a temperature of at least 60° C.

In some embodiments of any of the aspects, the heated, pressurized gas is outlet from the drying chamber at a temperature of at most 85° C.

In some embodiments of any of the aspects, the heated, pressurized gas is pressurized before being inlet into the drying chamber.

In some embodiments of any of the aspects, the heated, pressurized gas in the drying chamber has an atomization gas pressure of at least 10 pounds per square inch gauge (psig).

In some embodiments of any of the aspects, the heated, pressurized gas in the drying chamber has an atomization gas pressure of at least 20 pounds per square inch gauge (psig).

In some embodiments of any of the aspects, the heated, pressurized gas in the drying chamber has an atomization gas pressure of at most 150 pounds per square inch gauge (psig).

In some embodiments of any of the aspects, the heated, pressurized gas has a flow rate through the drying chamber of at least 5 kg/hr.

In some embodiments of any of the aspects, the heated, pressurized gas has a flow rate through the drying chamber of at least 18 kg/hr.

In some embodiments of any of the aspects, the heated, pressurized gas has a flow rate through the drying chamber of at most 150 kg/hr.

In some embodiments of any of the aspects, the heated, pressurized gas is outlet through the cyclone chamber.

In some embodiments of any of the aspects, the step of exposing the liquid feedstock droplets to heated, pressurized gas in the drying chamber takes at most 8 hours.

In some embodiments of any of the aspects, the dried particles isolated in the cyclone chamber have a median mass aerodynamic diameter (MMAD) of at least 1.5 μm to at most 7.5 μm.

In some embodiments of any of the aspects, the dried particles isolated in the cyclone chamber have a median mass aerodynamic diameter (MMAD) of at least 4.0 μm.

In some embodiments of any of the aspects, the dried particles isolated in the cyclone chamber have a median mass aerodynamic diameter (MMAD) of at most 5.0 μm.

In some embodiments of any of the aspects, the step of isolating dried particles of a predetermined range of diameters in the cyclone chamber occurs continuously.

In one aspect, described herein is a method of delivering a spray-dried pharmaceutical composition comprising a lactic acid-producing compound to a subject, comprising: (a) obtaining an inhalation device for bronchopulmonary delivery comprising: (i) an inhaler; and (ii) a container containing a spray-dried pharmaceutical composition comprising a lactic acid-producing compound; (b) activating the inhaler to cause aerosolization or dispersal of the spray-dried pharmaceutical composition; and (c) inhaling the aerosolized or dispersed spray-dried pharmaceutical composition.

In one aspect, described herein is a method of delivering a spray-dried pharmaceutical composition comprising a lactic acid-producing compound to a subject, comprising: (a) obtaining an inhalation device for bronchopulmonary delivery comprising: (i) an inhaler; and (ii) a container containing a pharmaceutical composition as described herein, wherein the pharmaceutical composition is spray-dried; (b) activating the inhaler to cause aerosolization or dispersal of the spray-dried pharmaceutical composition; and (c) inhaling the aerosolized or dispersed spray-dried pharmaceutical composition.

In one aspect, described herein is a method of delivering a spray-dried pharmaceutical composition comprising a lactic acid-producing compound to a subject, comprising: (a) obtaining an inhalation device as described herein; (b) activating the inhaler to cause aerosolization or dispersal of the spray-dried pharmaceutical composition; and (c) inhaling the aerosolized or dispersed spray-dried pharmaceutical composition.

In some embodiments of any of the aspects, the inhaler is a dry powder inhaler (DPI).

In some embodiments of any of the aspects, the inhaler is a metered dose inhaler (MDI).

In some embodiments of any of the aspects, the inhaler is a soft mist inhaler (SMI).

In some embodiments of any of the aspects, the inhaler comprises: (a) a mouthpiece comprising an opening; and (b) means for aerosolizing or dispersing the spray-dried pharmaceutical composition in the container.

In some embodiments of any of the aspects, the inhaler has an inspiration flow rate of at least 15 L/min to at most 60 L/min.

In some embodiments of any of the aspects, at least 25% to at most 125% of the spray-dried pharmaceutical composition by mass is delivered to a target bronchopulmonary tissue.

In some embodiments of any of the aspects, at least 30% of the spray-dried pharmaceutical composition by mass is delivered to a target bronchopulmonary tissue.

In some embodiments of any of the aspects, the target bronchopulmonary tissue is the lungs, the trachea, the bronchi, the bronchioles, and/or the alveoli.

In some embodiments of any of the aspects, the spray-dried pharmaceutical composition is delivered from the bronchopulmonary tissue to a distal tissue site via the cardiovascular system or lymphatic system.

In one aspect, described herein is a method of treating a subject in need thereof comprising administering through inhalation an effective dose of a pharmaceutical composition comprising a lactic acid-producing compound.

In one aspect, described herein is a method of treating a subject in need thereof comprising administering through inhalation an effective dose of a pharmaceutical composition as described herein.

In some embodiments of any of the aspects, the lactic acid-producing compound reduces neutrophilic inflammation in a target tissue.

In some embodiments of any of the aspects, the target tissue is a target bronchopulmonary tissue.

In some embodiments of any of the aspects, the target bronchopulmonary tissue is the lungs, the trachea, the bronchi, the bronchioles, and/or the alveoli.

In some embodiments of any of the aspects, the target tissue is a distal tissue site from the lungs delivered via the cardiovascular system or lymphatic system.

In some embodiments of any of the aspects, the subject has been diagnosed with or is at risk of developing a chronic or infectious bronchopulmonary disease.

In some embodiments of any of the aspects, the chronic bronchopulmonary disease is selected from the group consisting of: asthma, bronchopulmonary dysplasia (BPD), chronic obstructive pulmonary disease (COPD), bronchiectasis, non-cystic fibrosis (CF) bronchiectasis, cystic fibrosis (CF), acute respiratory distress syndrome (ARDS), idiopathic pulmonary fibrosis (IPF), interstitial lung disease (LD), pleural effusion (PE), pulmonary hypertension (PAH), silicosis, and lung cancer.

In some embodiments of any of the aspects, the lung cancer is small cell lung cancer (SCLC) or non-small cell lung cancer (NSCLC).

In some embodiments of any of the aspects, the infectious bronchopulmonary disease is caused by or associated with an infectious agent selected from: adenovirus; coronavirus; influenza virus; parainfluenza virus; parvovirus; respiratory syncytial virus; rhinovirus; enterovirus; measles virus; rubella virus; varicella virus; Corynebacterium diphtheriae; Haemophilus influenzae; Legionella pneumophila; Bordetella pertussis; Mycobacterium tuberculosis; Streptococcus species; Pseudomonas species; Escherichia coli; Aspergillus species; Cryptococcus species; and Pneumocystis species.

In some embodiments of any of the aspects, the pharmaceutical composition is a spray-dried pharmaceutical composition.

In some embodiments of any of the aspects, the pharmaceutical composition is administered using the standard of care for the chronic or infectious bronchopulmonary disease.

In some embodiments of any of the aspects, the pharmaceutical composition is administered using an inhaler.

In some embodiments of any of the aspects, the inhaler is a dry powder inhaler (DPI).

In some embodiments of any of the aspects, the inhaler is a metered dose inhaler (MDI).

In some embodiments of any of the aspects, the inhaler is a soft mist inhaler (SMI).

In some embodiments of any of the aspects, the effective dose of the pharmaceutical composition is at least 7.8 mg lactic acid-producing compound per unit dose.

In some embodiments of any of the aspects, the pharmaceutical composition is co-administered with at least one additional therapeutic for a chronic or infectious bronchopulmonary disorder.

In some embodiments of any of the aspects, the at least one additional therapeutic is an anti-inflammatory, an antimicrobial, an antiviral, an antifungal, a vasodilator, or a bronchodilator.

In one aspect, described herein is a unit dosage form comprising at least 1.0 mg to at most 100.0 mg of a pharmaceutical composition comprising a lactic acid-producing compound.

In one aspect, described herein is a unit dosage form comprising at least 1.0 mg to at most 100.0 mg of a pharmaceutical composition as described herein.

In one aspect, described herein is a unit dosage form comprising at least 1.0 mg to at most 100.0 mg of the spray-dried pharmaceutical composition prepared by a method as described herein.

In one aspect, described herein is a unit dosage form comprising at least 15.0 mg to at most 100.0 mg of a pharmaceutical composition comprising at least 15 mg lactic acid-producing compound per unit dose.

In some embodiments of any of the aspects, the dosage is at least 1.0 mg pharmaceutical composition.

In some embodiments of any of the aspects, the dosage comprises at least 7.8 mg lactic acid-producing compound per unit dose that is deliverable to a target tissue.

In some embodiments of any of the aspects, the dosage comprises at least 15 mg lactic acid-producing compound per unit dose.

In some embodiments of any of the aspects, the pharmaceutical composition is a spray-dried pharmaceutical composition.

In one aspect, described herein is a pharmaceutical composition comprising: (a) a lactic acid-producing compound; and (b) a pharmaceutically acceptable excipient, stabilizer, or additive; wherein the composition is formulated for oral administration.

In some embodiments of any of the aspects, the lactic acid-producing compound is selected from: (a) a polymeric compound that can produce lactic acid; (b) a non-polymeric compound that can produce lactic acid; or (c) lactic acid.

In some embodiments of any of the aspects, the non-polymeric lactic acid-producing compound is an inorganic salt of lactic acid, an ester of lactic acid, or lactide.

In some embodiments of any of the aspects, the polymeric lactic acid-producing compound is a polylactic acid (PLA).

In some embodiments of any of the aspects, the polylactic acid is poly(L-lactide) (PLLA), poly(D,L-lactide) (PDLLA), or poly(D-lactide) (PDLA).

In some embodiments of any of the aspects, the composition comprises at least one excipient or at least one stabilizer.

In some embodiments of any of the aspects, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments of any of the aspects, the excipient is leucine and/or trehalose.

In some embodiments of any of the aspects, the stabilizer is a polysorbate; poloxamer; or polyvinyl alcohol.

In some embodiments of any of the aspects, the stabilizer is Poloxamer 188.

In some embodiments of any of the aspects, the additive is an adhesion agent.

In some embodiments of any of the aspects, the composition comprises a plurality of dried particles.

In some embodiments of any of the aspects, the composition comprises a plurality of spray-dried particles.

In some embodiments of any of the aspects, the composition is formulated as a capsule or a tablet.

In one aspect, described herein is a method of treating a subject in need thereof comprising orally administering an effective dose of a pharmaceutical composition comprising a lactic acid-producing compound.

In one aspect, described herein is a method of treating a subject in need thereof comprising orally an effective dose of a pharmaceutical composition as described herein.

In some embodiments of any of the aspects, the lactic acid-producing compound reduces neutrophilic inflammation in a target tissue.

In some embodiments of any of the aspects, the target tissue is a bronchopulmonary target tissue.

In some embodiments of any of the aspects, the subject has been diagnosed with or is at risk of developing a chronic or infectious bronchopulmonary disorder.

In some embodiments of any of the aspects, the pharmaceutical composition is a spray-dried pharmaceutical composition.

In some embodiments of any of the aspects, the pharmaceutical composition is co-administered with at least one additional therapeutic.

In some embodiments of any of the aspects, the at least one additional therapeutic is an anti-inflammatory, an antimicrobial, a vasodilator, or a bronchodilator.

### BRIEF DESCRIPTION OF THE DRAWINGS

FIG. 1A-1B is a series of bar graphs showing lactic acid production of the bacterial strains AB101, AB102, AB103. FIG. 1A shows the production of L(+)-lactic acid in supernatant of each strain or a blend thereof. FIG. 1B shows the production of D(−)-lactic acid in supernatant of each strain or a blend thereof.

FIG. 2 is a bar graph showing that lactic acid in increasing concentrations decreased Matrix Metalloproteinase-9 (MMP-9) mRNA levels in a dysbiosis model of lung epithelial cells (human primary epithelial (HBE) cells treated with E. coli).

FIG. 3 is a bar graph showing that MMP-9 mRNA levels decreased in human intestinal epithelial cells (IECs) from the Caco-2 cell line that were inoculated with E. coli and then treated with varying concentrations of L-lactic acid (L-LA).

FIG. 4 is a dot plot showing that MMP-9 mRNA levels decreased in A549 non-small cell lung cancer (NSCLC) adenocarcinoma cells inoculated with E. coli and then treated with 0.25 μg to 4 ug L-lactic acid (L-LA).

FIG. 5 is a dot plot showing MMP-9 mRNA levels in human bronchial epithelial (HBE) cells inoculated with E. coli and then treated with 0.25 μg to 2 ug D-lactic acid (D-LA); no statistical difference in MMP9 mRNA levels was seen with D-LA treatment.

FIG. 6 is a bar graph showing L-lactic acid production of the bacterial strains AB101, AB102, or AB103 in vitro. The left-right order of the bars corresponds to the top-down order of the graph legend.

FIG. 7 is a line graph showing the hydrolysis of Poly-Lactic Acid powder (PLA) in a phosphate-buffered saline (PBS) solution into L(+) Lactic Acid.

### DETAILED DESCRIPTION

The technology described herein is directed to pharmaceutical compositions comprising a lactic acid-producing compound, which is formulated for administration by inhalation and/or oral administration. Also described herein are unit dosage forms of such pharmaceutical compositions, devices comprising such pharmaceutical compositions, methods of producing such pharmaceutical compositions, and methods of treating bronchopulmonary disease, among others, using such pharmaceutical compositions.

Providing a dose of a lactic acid-producing compound to the lungs of a chronic disease sufferer can have significant benefits to the patient, especially as it relates to dysbiosis. Dysbiosis occurs when there is an imbalance of commensal (beneficial) and pathogenic (harmful) bacteria. Data indicate that active bacterial extracts and metabolites can produce anti-inflammatory effects systemically. See e.g., Jang et al. (2020) Experimental & Molecular Medicine 52 (7):1128-1139; Weingarden and Vaughn B P (2017) Gut Microbes 8 (3):238-252; Arpaia et al. (2013) Nature 504 (7480):451-455; Iraporda et al. (2015) Immunobiology 220 (10):1161-1169; Vinolo et al. (2011) Nutrients 3 (10):858-876; the contents of each of which are incorporated herein by reference in their entireties. As described herein, it has been discovered that lactic acid itself is an active element or principle in such bacterial extracts and metabolites, such that delivery of lactic acid can provide therapeutic benefit.

### Preparation and Delivery Formats for Lactic Acid Therapeutics

Delivery of a lactic acid-producing compound directly to the lungs requires an inhaled dosing technique, such as nebulized delivery, a metered dose inhaler (MDI), a dry powder inhaler (DPI), or a soft mist inhaler (SMI). Dosing live biotherapeutics, in particular, is challenging due to stability concerns. The presence of moisture makes storage and use of a powdered biotherapeutic a challenge. Stability in this case would also be a concern for biotherapeutics, such as lactic acid-producing compounds, that have been carefully extracted and can require gentle processing.

To avoid these challenges, an inhalable dry powder through a DPI can be used. However, a therapeutic must be formulated as an inhalable dry powder to be used in a DPI successfully. This powder must possess specific moisture contents (usually very low, between 1 and 5%) for stability purposes, as well as particular aerodynamic properties to ensure the powder can be delivered properly and reliably. A process known as spray drying can be used to “engineer” this type of dry powder for use in a DPI.

### Spray Drying

Spray drying is a technique through which multiple raw materials are dissolved, atomized into droplets, and dried quickly in a drying chamber to create a custom, dry, aerosolize-able powder with tuned particle characteristics. It can be used for production of inhaled dry powders to treat diseases such as COPD, cystic fibrosis (CF), asthma, as well as non-respiratory disorders such as diabetes and migraines.

Spray drying involves the creation of a feedstock containing dissolved solid ingredients intended to be present in the dry particle at some defined concentration (e.g., a percent ratio of solute weight to solvent weight, % w/w). The feedstock is then fed through a nozzle at a specified pressure to create a droplet. That droplet is dried by heated gas running through the drying chamber to quickly create a dry particle. That particle is then collected at the bottom of a cyclone—a device designed to capture a reduced range of particle diameters, letting the rest of the “waste” particles be collected at the end of the process. These cyclone-captured particles represent a drug product, intended or designed to be inhaled for the treatment or prevention of disease.

The production of an effective spray-dried powder can include more components than simply the active pharmaceutical ingredient itself. The powder can include the active ingredient, one or more excipients, residual solvent, and/or an emulsification stabilizer. These ingredients are dissolved or suspended in the feedstock to be dried prior to the start of spray drying. The homogenous solution or suspension is then dried to create the inhalable dry powder itself.

The included excipients are used to provide a number of benefits to the spray dried powder. Namely, they are included to provide specific thermodynamic and physical properties. The excipients are often responsible for the shape of the spray dried particle itself due to their solubility properties. In the feedstock solvent, the solubility of the excipient determines how quickly the sprayed droplet forms a solid particle, and how quickly the solid molecules move toward the center of the droplet during drying. Once the particle is dried, the chemical and thermodynamic characteristics of the powder and their stability affect the solubility of the final powder, namely through the polymorphism (or lack thereof) of the final powder over time and across temperature and humidity exposures. Good excipient selection results in a dry powder exhibiting stable crystallinity, high particle density, consistent shape, and high dispersibility. Frequently, amino acids such as leucine are combined with a salt or sugar to optimize for this effect.

Surfactant stabilizers are frequently used when spray drying formulations with hydrophobic or insoluble particles. In some embodiments, the formulation includes a stabilizer when spray drying a suspension so that the suspension emulsifies and disperses evenly, allowing for a homogeneous suspension from which to spray consistent droplets. These surfactants are often included at low rates to minimally affect the final dry powder. Polysorbates of varying purity are frequently used, but other organic acid combinations are possible, along with steric surfactants, such as PLURONIC F68.

These spray dried powders can be encapsulated and used in some form of dry powder delivery device through which a patient inhales the particles deeply, allowing for settling into target depths in the airways. These spray dried powders can be filled into break-able capsules or sachets and broken open at the time of use inside of the delivery device. Once the enclosure is broken, the patient inhales deeply through the mouthpiece on their inhaler, allowing the powder to deposit into the patient's throat, esophagus, and lungs. The intention of a dry powder for inhalation is to create a powder of a certain aerodynamic size and density such that a predictable, safe dose of drug deposits in the appropriate portion of the lung airway.

Described herein is a drug delivery mechanism by which a patient can dose their lungs directly with a lactic acid-producing compound via inhalation of a spray dried powder for the treatment of a chronic lung disease marked by inflammation.

### Pharmaceutical Compositions

Described herein are pharmaceutical compositions comprising a lactic acid-producing compound and a pharmaceutically acceptable excipient, stabilizer, or additive. In some embodiments, the lactic acid-producing compound is selected from: a polymeric compound that can produce lactic acid; a non-polymeric compound that can produce lactic acid; or lactic acid. In some embodiments, the lactic acid-producing compound is a derivative, polymer, or intermediate of lactic acid. In some embodiments, the pharmaceutical composition is formulated for administration to the lungs (e.g., by inhalation). In some embodiments, the pharmaceutical composition is formulated for administration by inhalation. In some embodiments, the pharmaceutical composition is formulated for oral administration. The spray-drying methods as described further herein can be used to prepare a pharmaceutical composition is formulated for inhalation and/or oral administration.

In one aspect, described herein is a pharmaceutical composition comprising: (a) a lactic acid-producing compound selected from: (i) a polymeric compound that can produce lactic acid; (ii) a non-polymeric compound that can produce lactic acid; or (iii) lactic acid; and (b) a pharmaceutically acceptable excipient, stabilizer, or additive; wherein the composition is formulated for administration by inhalation.

In one aspect, described herein is a pharmaceutical composition comprising: (a) lactic acid; and (b) a pharmaceutically acceptable excipient, stabilizer, or additive; wherein the composition is formulated for administration by inhalation. In one aspect, described herein is a pharmaceutical composition comprising: (a) a non-polymeric compound that can produce lactic acid; and (b) a pharmaceutically acceptable excipient, stabilizer, or additive; wherein the composition is formulated for administration by inhalation. In one aspect, described herein is a pharmaceutical composition comprising: (a) a polymeric compound that can produce lactic acid; and (b) a pharmaceutically acceptable excipient, stabilizer, or additive; wherein the composition is formulated for administration by inhalation. In some embodiments, the composition comprises at least one excipient and at least stabilizer, as described further herein (see e.g., Table 1)

TABLE 1
Exemplary pharmaceutical composition formulated for administration
by inhalation, 30 mg capsule fill dosed (e.g., dry powder for DPI)

| Constituent | % by weight | mg |
|---|---|---|
| Lactic acid-producing compound | 50.00% | 15.00 |
| Excipient 1 | 23.75% | 7.125 |
| Excipient 2 | 23.75% | 7.125 |
| Stabilizer | 1.50% | 0.450 |

TABLE 11
Minimum constituent concentrations in an exemplary pharmaceutical
composition (e.g., for administration by inhalation; each
constituent represents an exemplary minimum % value that
can be made up for the other constituents in the composition;
e.g., a low % of lactic acid-producing compound can be
counteracted by higher than minimum percentage of excipient
1, excipient 2, and/or stabilizer).

| Constituent | % by weight |
|---|---|
| Lactic acid-producing compound | 1.0 |
| Excipient 1 | 5.0 |
| Excipient 2 | 5.0 |
| Stabilizer | 0.10% |

In some embodiments, the lactic acid-producing compound produces lactic acid upon delivery to a target tissue. In some embodiments, for example when the pharmaceutical composition is formulated for administration by inhalation and/or administration to the lungs, the target tissue is a target bronchopulmonary tissue. In some embodiments, the target bronchopulmonary tissue is the lungs, the trachea, the bronchi, the bronchioles, and/or the alveoli. In some embodiments, the target bronchopulmonary tissue is the lungs. In some embodiments, the target bronchopulmonary tissue is the trachea. In some embodiments, the target bronchopulmonary tissue is the bronchi. In some embodiments, the target bronchopulmonary tissue is the bronchioles. In some embodiments, the target bronchopulmonary tissue is the alveoli. In some embodiments, the target tissue is a distal tissue site from the lungs. In some embodiments, the composition is delivered to a distal target site via the cardiovascular system or lymphatic system.

In one aspect, described herein is a pharmaceutical composition comprising: (a) a lactic acid-producing compound selected from: (i) a polymeric compound that can produce lactic acid; (ii) a non-polymeric compound that can produce lactic acid; or (iii) lactic acid; and (b) a pharmaceutically acceptable excipient, stabilizer, or additive; wherein the composition is formulated for oral administration. In some embodiments, the additive is an adhesion agent, e.g., for oral formulation.

In one aspect, described herein is a pharmaceutical composition comprising: (a) lactic acid; and (b) a pharmaceutically acceptable excipient, stabilizer, or additive; wherein the composition is formulated for oral administration. In one aspect, described herein is a pharmaceutical composition comprising: (a) a non-polymeric compound that can produce lactic acid; and (b) a pharmaceutically acceptable excipient, stabilizer, or additive; wherein the composition is formulated for oral administration. In one aspect, described herein is a pharmaceutical composition comprising: (a) a polymeric compound that can produce lactic acid; and (b) a pharmaceutically acceptable excipient, stabilizer, or additive; wherein the composition is formulated for oral administration. In some embodiments, the composition comprises at least one excipient, at least one stabilizer, and at least one additive (e.g., an adhesion agent) as described further herein (see e.g., Table 2).

In one aspect, described herein is a pharmaceutical composition comprising: (a) a lactic acid-producing compound selected from: (i) a polymeric compound that can produce lactic acid; (ii) a non-polymeric compound that can produce lactic acid; or (iii) lactic acid; (b) at least one excipient; (c) at least one stabilizer; and (d) at least one adhesion agent; wherein the composition is formulated for oral administration. The pharmaceutical compositions formulated for oral administration described herein can be produced by first spray-drying and then tableting the spray-dried matrix; such a method allows for modulation of the stability characteristics of the spray-dried matrix as well as the size of the active ingredient (e.g., a lactic acid-producing compound) itself in the tableted matrix.

TABLE 2
Exemplary pharmaceutical composition
formulated for oral administration

| Constituent | % by weight | mg |
|---|---|---|
| Lactic acid-producing compound | 25.0% | 50.0 |
| Excipient 1 | 36.0% | 72.0 |
| Excipient 2 | 36.0% | 72.0 |
| Stabilizer | 1.50% | 3.00 |
| Adhesion agent | 1.50% | 3.00 |

### Lactic Acid-Producing Compounds

The pharmaceutical compositions as described herein comprise at least one lactic acid-producing compound. As used herein, the term “lactic acid-producing compound” refers to a compound that produces lactic acid, either because it comprises lactic acid or lactic acid is produced when the compound is hydrolyzed, metabolized, or otherwise chemically altered. In some embodiments, the pharmaceutical composition comprises at least 1, 2, 3, 4, 5, 6, 7, 8, 9, 10, or more lactic acid-producing compounds. In some embodiments, the lactic acid-producing compound is selected from: (i) lactic acid; (ii) a non-polymeric compound that can produce lactic acid; or (iii) a polymeric compound that can produce lactic acid.

In some embodiments, the pharmaceutical composition comprises: (i) lactic acid. In some embodiments, the pharmaceutical composition comprises: (ii) a non-polymeric compound that can produce lactic acid. In some embodiments, the pharmaceutical composition comprises: (iii) a polymeric compound that can produce lactic acid. In some embodiments, the pharmaceutical composition comprises: (i) lactic acid; and (ii) a non-polymeric compound that can produce lactic acid. In some embodiments, the pharmaceutical composition comprises: (i) lactic acid; and (iii) a polymeric compound that can produce lactic acid. In some embodiments, the pharmaceutical composition comprises: (ii) a non-polymeric compound that can produce lactic acid; and (iii) a polymeric compound that can produce lactic acid. In some embodiments, the pharmaceutical composition comprises: (i) lactic acid; (ii) a non-polymeric compound that can produce lactic acid; and (iii) a polymeric compound that can produce lactic acid.

Lactic acid is a byproduct of metabolism. In animals, L-lactate is produced from pyruvate via the enzyme lactate dehydrogenase (LDH) in a process of fermentation during normal metabolism and exercise. The concentration of human blood lactate is usually 1 mM-2 mM at rest, but can rise to over 20 mM during intense exertion and as high as 25 mM afterward. In addition to other biological roles, L-lactic acid is the primary endogenous agonist of hydroxycarboxylic acid receptor 1 (HCA1), which is a G_{i/o}-coupled G protein-coupled receptor (GPCR). Lactic acid fermentation can also be performed by lactic acid bacteria, which convert simple carbohydrates such as glucose, sucrose, or galactose to lactic acid.

Lactic acid is a chiral compound, consisting of two enantiomers. One enantiomer is known as L-lactic acid, (S)-lactic acid, or (+)-lactic acid (see e.g., Formula I, below), and the other enantiomer, its mirror image, is D-lactic acid, (R)-lactic acid, or (−)-lactic acid (see e.g., Formula II, below). A mixture of the two enantiomers, e.g., in equal amounts, is called DL-lactic acid, or racemic lactic acid. As used, here “racemic mixture” refers to a solution in which there is 50:50 ratio of both enantiomers of a compound. Contemplated herein is aerosol delivery into the lungs of both the D or L form as well as the DL racemic mix.

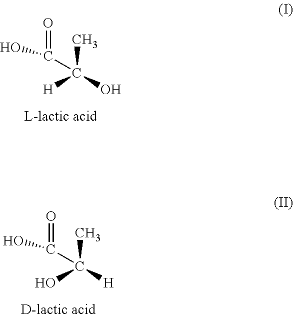

Both L and D enantiomers of lactic acid have advantages. L-lactic acid is the naturally-occurring enantiomer (e.g., in humans). D-lactic acid can be bioavailable for longer periods, e.g., as compared to L-lactic acid. Described herein are methods of inhaled delivery of such lactic acid enantiomers; it is contemplated herein that altering the frequency, dose, and/or delivery device (e.g., metered dose inhalers (MDIs) can optimize or tailor therapeutic effects, e.g., for certain indications. D, L, and racemic mixtures may also be used in oral tablet or capsule formulations.

Accordingly, in some embodiments, the lactic acid-producing compound comprises a D enantiomer of lactic acid, an L enantiomer of lactic acid, or a racemic mixture of D and L enantiomers of lactic acid. In some embodiments, the lactic acid-producing compound comprises a D enantiomer of lactic acid. In some embodiments, the lactic acid-producing compound comprises an L enantiomer of lactic acid. In some embodiments, the lactic acid-producing compound comprises a racemic mixture of D and L enantiomers of lactic acid.

In some embodiments, the lactic acid-producing compound is a non-polymeric compound that can produce lactic acid. As used herein, the term “non-polymeric compound” refers to a molecule that does not comprise repeating monomers. In some embodiments, the non-polymeric lactic acid-producing compound can be metabolized in the target tissue to produce lactic acid. In some embodiments, the non-polymeric lactic acid-producing compound can be metabolized using hydrolysis in the target tissue to produce lactic acid. In some embodiments, the non-polymeric lactic acid-producing compound can be completely metabolized (i.e., all of the compound is broken down into lactic acid and other byproducts) or partially metabolized (i.e., at least a portion of the compound is broken down into lactic acid and other byproducts) in the target tissue.

In some embodiments, the non-polymeric lactic acid-producing compound is an inorganic salt of lactic acid, an ester of lactic acid, or lactide. In some embodiments, the pharmaceutical composition comprises an inorganic salt of lactic acid, an ester of lactic acid, or lactide. In some embodiments, the pharmaceutical composition comprises an inorganic salt of lactic acid. In some embodiments, the pharmaceutical composition comprises an ester of lactic acid. In some embodiments, the pharmaceutical composition comprises lactide. In some embodiments, the pharmaceutical composition comprises an inorganic salt of lactic acid and an ester of lactic acid. In some embodiments, the pharmaceutical composition comprises an inorganic salt of lactic acid and lactide. In some embodiments, the pharmaceutical composition comprises an ester of lactic acid and lactide. In some embodiments, the pharmaceutical composition comprises an inorganic salt of lactic acid, an ester of lactic acid, and lactide.

In some embodiments, the non-polymeric lactic acid-producing compound is an inorganic salt of lactic acid. In this approach, rather than deliver lactic acid itself, a simple salt, such as sodium lactate, is delivered. One advantage of such an inorganic salt is that it is slowly converted to lactic acid and thus does not immediately lower the pH (i.e., through increased levels of lactic acid) of the microenvironment in the target tissue. In some embodiments, the inorganic salt of lactic acid is sodium lactate (NaC₃H₅O₃). In some embodiments, the inorganic salt of lactic acid is potassium lactate (KC₃H₅O₃). In some embodiments, the inorganic salt of lactic acid is calcium lactate (Ca(C₃H₅O₃)₂). In some embodiments, the inorganic salt of lactic acid is magnesium lactate (Mg(C₃H₅O₃)₂).

In some embodiments, the non-polymeric lactic acid-producing compound is an ester of lactic acid. Esters of lactic acid can be hydrolyzed to generate lactic acid in vivo. The range of esters can include the water-soluble ethyl lactate to the hydrophobic dodecyl lactate. A range of compounds can be used that can form hydrolyzable linkages to lactic acid including esters and amines. In some embodiments, the ester of lactic acid is ethyl lactate (CH₃CH(OH)CO₂CH₂CH₃). In some embodiments, the ester of lactic acid is dodecyl lactate (CH₃CH(OH)CO₂CH₂CH₂CH₂CH₂CH₂CH₂CH₂CH₂CH₂CH₂CH₂CH₃). In some embodiments, the ester of lactic acid is selected from ethyl lactate, propyl lactate, butyl lactate, pentyl lactate, hexyl lactate, heptyl lactate, octyl lactate, nonyl lactate, decyl lactate, undecyl lactate, or dodecyl lactate.

In some embodiments, the non-polymeric lactic acid-producing compound is lactide. Lactide is a cyclic dimer formed from lactic acid (see e.g., Formula III, below). Both L and D enantiomeric forms of lactide, or racemic mixtures thereof, can be used. Lactide compounds are stable while dry but generate two molecules of lactic acid upon hydrolysis in vivo.

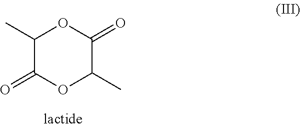

In some embodiments, a pharmaceutical composition comprising at least one non-polymeric lactic acid-producing compound further comprises an additive. In some embodiments, the additive is a buffer salt or surfactant. In some embodiments, the additive allows for encapsulation. For example, lactic acid can be encapsulated into polyethylene glycol (PEG) or polyvinylpyrrolidone (PVP) capsules, which allows for easier administration. Such capsules can dissolve upon administration, allowing for deeper penetration, long duration, and slower pH change from the administered lactic acid-producing compound.

In some embodiments, the lactic acid-producing compound is a polymeric compound that can produce lactic acid. As used herein, the term “polymeric compound” refers to a molecule comprising repeating monomers. In some embodiments, at least one of the monomers in the polymeric compound comprises lactic acid or a derivative thereof. In some embodiments, the polymeric lactic acid-producing compound can be hydrolyzed in the target tissue to produce lactic acid; as such hydrolysis occurs wherever this is water, such a step does not require an enzyme. In some embodiments, the polymeric lactic acid-producing compound can be completely hydrolyzed (i.e., all of the compound is broken down into lactic acid and other byproducts) or partially hydrolyzed (i.e., at least a portion of the compound is broken down into lactic acid and other byproducts) in the target tissue.

The use of biodegradable polymers based on hydroxy acids is known in the art. Uses include a range of medical device indications where the polymer performs a mechanical use, to forms which are the basis of pharmaceutical drug delivery, where the polymer is used to sustain delivery over a period of days to months. In such cases, the polymer degrades to the corresponding hydroxy acid during or after its functional use or administration. Examples include both poly (L-lactide, poly (DL-lactide) and copolymers of these with glycolic acid (a family generally abbreviated as PLGA) and with caprolactone. Several deliverable forms of polymeric lactic acid-producing compound are possible. In some embodiments, the polymeric lactic acid-producing compound comprises a particulate form of poly-lactic acid, prepared for example by milling to a particular particle size. Controlled variables can include particle size, polymer molecular weight (MW), chirality, and porosity.

In some embodiments, the polymeric lactic acid-producing compound is a polylactic acid (PLA) (see e.g., Formula IV, below). Polylactic acid, also known as poly(lactic acid) or polylactide is a thermoplastic polyester with backbone formula (C₃H₄O₂), or [—C(CH₃)HC(═O)O—]ₙ, formally obtained by condensation of lactic acid C(CH₃)(OH)HCOOH with loss of water. PLA can also be prepared by ring-opening polymerization of lactide [—C(CH₃)HC(═O)O—]₂, the cyclic dimer of the basic repeating unit. In some embodiments, the PLA polymer comprises at least 2 monomers, at least 3 monomers, at least 4 monomers, at least 5 monomers, at least 10 monomers, at least 50 monomers, at least 100 monomers, at least 10³ monomers, at least 10⁴ monomers, at least 10⁵ monomers, at least 10⁶ monomers, at least 10⁷ monomers, at least 10⁸ monomers, at least 10⁹ monomers, at least 10¹⁰ monomers or more of lactic acid.

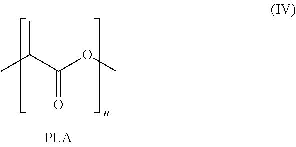

In some embodiments, the polylactic acid is poly(L-lactide) (PLLA), poly(D,L-lactide) (PDLLA), or poly(D-lactide) (PDLA). In some embodiments, the pharmaceutical composition comprises PLLA, PDLLA, or PDLA. In some embodiments, the pharmaceutical composition comprises PLLA. In some embodiments, the pharmaceutical composition comprises PDLLA. In some embodiments, the pharmaceutical composition comprises PDLA. In some embodiments, the pharmaceutical composition comprises PLLA and PDLLA. In some embodiments, the pharmaceutical composition comprises PLLA and PDLA. In some embodiments, the pharmaceutical composition comprises PDLLA and PDLA. In some embodiments, the pharmaceutical composition comprises PLLA, PDLLA, and PDLA. In some embodiments, the polylactic acid is poly(L-lactide) (PLLA). In some embodiments, the polylactic acid is poly(D-lactide) (PDLA). In some embodiments, the polylactic acid is poly(D,L-lactide) (PDLLA).

In some embodiments, the polymeric lactic acid-producing compound is poly(lactic-co-glycolic acid) (PLGA) (see e.g., Formula V, below). PLGA, PLG, or poly(lactic-co-glycolic acid) is a copolymer has high biodegradability and biocompatibility. In some embodiments, the PLGA polymer comprises at least 2 monomers, at least 3 monomers, at least 4 monomers, at least 5 monomers, at least 10 monomers, at least 50 monomers, at least 100 monomers, at least 10³ monomers, at least 10⁴ monomers, at least 10⁵ monomers, at least 10⁶ monomers, at least 10⁷ monomers, at least 10⁸ monomers, at least 10⁹ monomers, at least 10¹⁰ monomers or more of lactic acid.

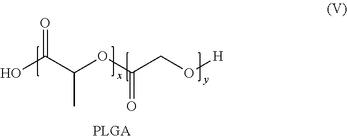

PLGA is synthesized through ring-opening co-polymerization of two different monomers, the cyclic dimers (e.g., 1,4-dioxane-2,5-diones) of glycolic acid and lactic acid. PLGA polymers can be synthesized as either random or block copolymers thereby imparting additional polymer properties. Common catalysts used in the preparation of the PLGA polymer include tin(II) 2-ethylhexanoate, tin(II) alkoxides, or aluminum isopropoxide. During polymerization, successive monomeric units (of glycolic or lactic acid) are linked together in PLGA by ester linkages, thus yielding a linear, aliphatic polyester as a product.

Depending on the ratio of lactide to glycolide used for the polymerization, different forms of PLGA can be obtained: these are usually identified in regard to the molar ratio of the monomers used (e.g. PLGA 75:25 identifies a copolymer whose composition is 75% lactic acid and 25% glycolic acid). In some embodiments, the pharmaceutical composition comprises: PLGA 10:90, PLGA 20:80, PLGA 25:75, PLGA 30:70, PLGA 40:60, PLGA 50:50, PLGA 60:40, PLGA 70:30, PLGA 75:25, PLGA 80:20, PLGA 90:10, of lactic acid to glycolic acid. The crystallinity of PLGAs varies from fully amorphous to fully crystalline depending on block structure and molar ratio. PLGAs typically show a glass transition temperature in the range of 40-60° C.

PLGA undergoes hydrolysis in the body to produce the original monomers: lactic acid and glycolic acid. PLGA degrades by hydrolysis of its ester linkages in the presence of water. PLGA materials can be tailored to degrade more quickly than a PLA homopolymer. The time required for degradation of PLGA is related to the monomers' ratio used in production: the higher the content of glycolide units, the lower the time required for degradation as compared to predominantly lactide materials. An exception to this rule is the copolymer with 50:50 monomers' ratio which exhibits the faster degradation (about two months). In addition, polymers that are end-capped with esters (as opposed to the free carboxylic acid) demonstrate longer degradation half-lives. Microspheres can be used in drug delivery with PLGA, e.g., PLGA particles in the range of a few microns to 100s of microns in diameter.

In some embodiments, the polymeric lactic acid-producing compound comprises caprolactone (see e.g., Formula VI, below). In some embodiments, the polymeric lactic acid-producing compound is produced from caprolactone. ε-Caprolactone or simply caprolactone is a lactone (a cyclic ester) possessing a seven-membered ring. Its name is derived from caproic acid. This colorless liquid is miscible with most organic solvents and water. Several other caprolactones are known. These isomers include α-, β-, γ-, and δ-caprolactones, all of which are chiral. Caprolactone is prepared industrially by Baeyer-Villiger oxidation of cyclohexanone with peracetic acid. Ring-opening polymerization of caprolactone produces polycaprolactone.

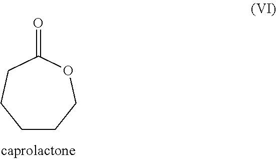

In some embodiments, the polymeric lactic acid-producing compound is poly(lactic acid-co-caprolactone) (PLCL; see e.g., Formula VII, below). In some embodiments, the polymeric lactic acid-producing compound is poly(D,L-lactide-co-caprolactone). In some embodiments, the poly(lactic acid-co-caprolactone) polymer comprises at least 2 monomers, at least 3 monomers, at least 4 monomers, at least 5 monomers, at least 10 monomers, at least 50 monomers, at least 100 monomers, at least 10³ monomers, at least 10⁴ monomers, at least 10⁵ monomers, at least 10⁶ monomers, at least 10⁷ monomers, at least 10⁸ monomers, at least 10⁹ monomers, at least 10¹⁰ monomers or more of lactic acid.

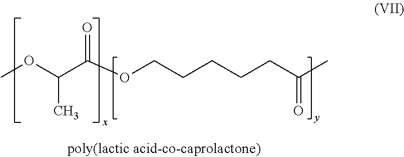

In some embodiments, the polymeric lactic acid-producing compound is poly(glycolic acid-epsilon-caprolactone) (PGCL) or polyglecaprone, which is a polymer of e-Caprolactone and glycolic acid. In some embodiments, the PGCL polymer comprises at least 2 monomers, at least 3 monomers, at least 4 monomers, at least 5 monomers, at least 10 monomers, at least 50 monomers, at least 100 monomers, at least 10³ monomers, at least 10⁴ monomers, at least 10⁵ monomers, at least 10⁶ monomers, at least 10⁷ monomers, at least 10⁸ monomers, at least 10⁹ monomers, at least 10¹⁰ monomers or more of lactic acid.

In some embodiments, the pharmaceutical composition comprises at least 1.0% lactic acid-producing compound by weight. In some embodiments, the pharmaceutical composition comprises at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more lactic acid-producing compound by weight.

In some embodiments, the pharmaceutical composition comprises at least 1.0 mg lactic acid-producing compound per unit dose. In some embodiments, the pharmaceutical composition comprises at least 5.0 mg lactic acid-producing compound per unit dose. In some embodiments, the pharmaceutical composition comprises at most 100.0 mg lactic acid-producing compound per unit dose. In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg lactic acid-producing compound per unit dose.

### Pharmaceutically Acceptable Excipients, Stabilizers, and Additives

In some embodiments, the technology described herein relates to a pharmaceutical composition comprising a lactic acid-producing compound as described herein, and optionally a pharmaceutically acceptable excipient, stabilizer, and/or additive. In some embodiments, the active ingredients of the pharmaceutical composition comprise a lactic acid-producing compound as described herein. In some embodiments, the active ingredients of the pharmaceutical composition consist essentially of a lactic acid-producing compound as described herein. In some embodiments, the active ingredients of the pharmaceutical composition consist of a lactic acid-producing compound as described herein. In some embodiments, pharmaceutically acceptable excipients, stabilizers, and/or additives include, for example, saline, aqueous buffer solutions, solvents and/or dispersion media. The use of such excipients, stabilizers, and/or additives is well known in the art. Some non-limiting examples of materials which can serve as pharmaceutically-acceptable excipients, stabilizers, and/or additives include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. In some embodiments, the pharmaceutically acceptable excipient, stabilizer, and/or additive inhibits the degradation of the active agent, e.g. a lactic acid-producing compound as described herein. In some embodiments, the composition comprises at least one pharmaceutically acceptable excipient and/or at least one pharmaceutically acceptable stabilizer.

### Excipients

In some embodiments, the pharmaceutical composition comprises at least one excipient. In some embodiments, the pharmaceutical composition comprises at least 1, 2, 3, 4, 5, 6, 7, 8, 9, 10, or more excipients. As used herein, the term “excipient” refers to an inactive substance that serves as the vehicle, diluent, or medium for the active substance, e.g., the lactic acid-producing compound as described herein. Excipients can ease processability of the pharmaceutical composition (e.g., formulated for respiratory or oral administration) and maintain physical structure of the pharmaceutical composition for added long term stability. In some embodiments, the pharmaceutical composition comprises at least two excipients. In some embodiments, the composition comprises two excipients. In some embodiments, the composition comprises three excipients. In some embodiments, the pharmaceutical composition comprises at least 2, at least 3, at least 4, at least 5, at least 6, at least 7, at least 8, at least 9, at least 10, or more excipients.

Typically, excipients are used when spray drying pharmaceutical drug products for two purposes: adding dispersibility to the final powder, and glass stabilization of crystalline or amorphous particles with high viscosity; see e.g., Vehring (2008) Pharm Res 25 (5):999-1022; the content of which is incorporated herein by reference in its entirety. Amino acids (e.g., leucine and trileucine) can be used for the effect of adding dispersibility to the final powder. A dispersible powder tends to clump together less readily, and tends to be more flowable in bulk, making encapsulation easier.

As spray drying creates a particle in an energetically unfavorable state, excipients used for glass stabilization are helpful in providing longer-term stability advantages and reliable solid-state chemical features, such as stable crystallinity or co-crystallinity leading to consistent bioavailability in vivo. The effectiveness of these excipients is typically evaluated by measuring the glass transition temperature of the dried particle, which is best maintained well above storage temperature (e.g., above −18° C. freezer, 4° C. refrigeration, or 20-22° C. room temperature) to ensure physical stability. In some embodiments, the minimum Tg (glass transition temperature) is 35° C. In some embodiments, the Tg (glass transition temperature) is at least 30° C., at least 35° C., at least 40° C., at least 45° C., or at least 50° C.

In some embodiments, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

In some embodiments, the excipient is selected from Table 9. In some embodiments, the excipient is selected from any combination of excipients listed in Table 9, e.g., a combination of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39 excipients from Table 9.

TABLE 9
Exemplary Excipients

| | | | |
|---|---|---|---|
| Excipients | Inulin | Glycine betaine | Starch |
| MRS growth medium | Sodium caseinate | Glycogen | Lactose |
| Gelatin | Goat's milk | Trehalose | Sucrose |
| Whey Isolate | Cow's milk | Mannose | Glucose |
| Sweet Whey | Proline | Xylose | Leucine and/or |
| | | | Trileucine |
| Reconstituted skim | Carnitine | Mannitol | Sodium salts |
| milk | | | |
| Maltodextrins | Acetylcarnitine | Sorbitol | Potassium salts |
| Gluco-oligosaccharides | Propionylcarnitine | Maltose | Lithium salts |
| Lacto-oligosaccharides | Glutamate | Dextrose | Calcium salts |
| Fructo-oligosaccharides | Sodium citrate | | |

In some embodiments, the excipient is leucine, trehalose, and/or sodium citrate. In some embodiments, the excipient is leucine and/or trehalose. In some embodiments, the excipient is leucine and/or sodium citrate. In some embodiments, the excipient is trehalose and/or sodium citrate.

In some embodiments, the excipient is leucine. In some embodiments, the excipient is L-leucine (see e.g., Formula VIII, below). In some embodiments, the excipient is D-leucine. In some embodiments, the excipient is a racemic mixture of L-leucine and D-leucine. Leucine (symbol Leu or L) is an essential amino acid for humans. In some embodiments, the excipient is trileucine (also referred to as Leu-Leu-Leu), which is a tripeptide composed of three leucine residues (see e.g., Formula IX, below). In some embodiments, trileucine is used in place of leucine as an excipient. In some embodiments, the excipient is trehalose see e.g., Formula X, below). Trehalose is a sugar consisting of two molecules of glucose. Trehalose is also known as mycose or tremalose. Trehalose has high water retention capabilities. Some bacteria, fungi, plants and invertebrate animals synthesize trehalose as a source of energy, and to survive freezing and lack of water. In some embodiments, the excipient is leucine and trehalose. In some embodiments, the first excipient is leucine and the second excipient is trehalose. In some embodiments, the second excipient is leucine and the first excipient is trehalose. In some embodiments, the excipient is sodium citrate (Na₃C₆H₅O₇; see e.g., Formula XI, below). In some embodiments, the first and second excipients are leucine and trehalose, and the third excipient is sodium citrate. In some embodiments, the three excipients are leucine, trehalose, and sodium citrate, and the stabilizer is polysorbate 80.

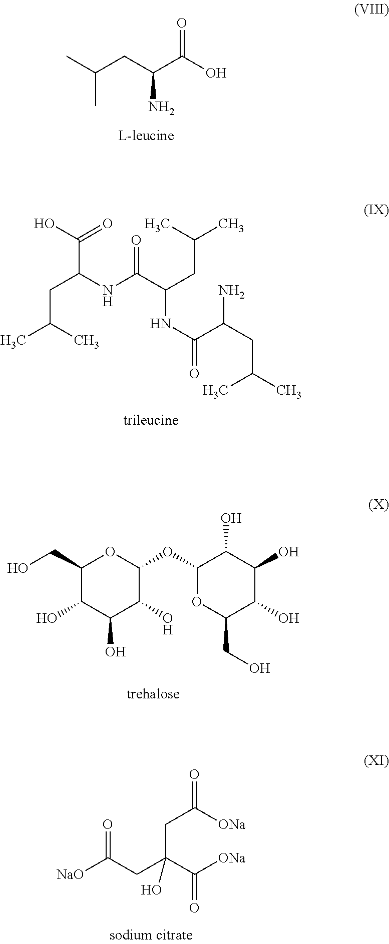

In some embodiments, the pharmaceutical composition comprises at least 5.0% excipient by weight. In some embodiments, the pharmaceutical composition comprises at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more excipient by weight.

In some embodiments, the pharmaceutical composition comprises at least 5.0% of a first excipient by weight, and at least 5.0% of a second excipient by weight. In some embodiments, the pharmaceutical composition comprises at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more of a first excipient by weight, and at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more of a second excipient by weight. In some embodiments, the pharmaceutical composition comprises at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more of a first excipient by weight, and at 5.0% of a second excipient by weight. In some embodiments, the pharmaceutical composition comprises at least 5.0% of a first excipient by weight, and at least 0.1%, at least 0.2%, at least 0.3%, at least 0.4%, at least 0.5%, at least 0.6%, at least 0.7%, at least 0.8%, at least 0.9%, at least 1.0%, at least 2.0%, at least 3.0%, at least 4.0%, at least 5.0%, at least 6.0%, at least 7.0%, at least 8.0%, at least 9.0%, at least 10.0% or more of a second excipient by weight.

In some embodiments, the pharmaceutical composition comprises at least 75 mg excipient per unit dose. In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more excipient per unit dose.

In some embodiments, the pharmaceutical composition comprises at least 75 mg of a first excipient, and at least 75 mg of a second excipient per unit dose. In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a first excipient, and at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a second excipient per unit dose.

In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a first excipient, and at least 75 mg of a second excipient per unit dose.

In some embodiments, the pharmaceutical composition comprises at least 75 mg of a first excipient, and at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 200 mg, at least 300 mg, at least 400 mg, at least 500 mg, or more of a second excipient per unit dose.

### Stabilizers

In some embodiments, the pharmaceutical composition comprises at least one stabilizer. In some embodiments, the pharmaceutical composition comprises at least 1, 2, 3, 4, 5, 6, 7, 8, 9, 10, or more stabilizers. As used herein, the term “stabilizer” refers to a substance which prevents or reduces the breakdown of the pharmaceutical composition. The use of stabilizers aids the process of wetting a hydrophobic substance to create a homogeneous suspension for spray drying. For example, a stabilizer can be included in case a wetting agent is needed to get any poorly soluble compounds into suspension. These stabilizers act as an emulsifier to enhance consistency across the bulk spray dried powder. In addition, some stabilizers (e.g., Poloxamers) can be used to pre-disperse polymers in feedstock solutions, even allowing for tunable wetted particle sizing down to the nanoscale; see e.g., Da Silva et al. (2019) Front Bioeng Biotechnol 7:137; the content of which is incorporated herein by reference in its entirety.

In some embodiments, the stabilizer comprises a surfactant. A surfactant is a substance which tends to reduce the surface tension of a liquid in which it is dissolved. In some embodiments, the stabilizer is selected from the group consisting of: mannitol, carboxymethyl cellulose (CMC), polyvinyl alcohol (PVA), polysorbate, and poloxamer. In some embodiments, the stabilizer is a polysorbate, poloxamer, or polyvinyl alcohol.

In some embodiments, the stabilizer is a Polysorbate. Polysorbate is a synthetic nonionic surfactant and emulsifier. Polysorbate is also referred to as MONTANOX; ALKEST TW; TWEEN; or PS. In some embodiments, the stabilizer is Polysorbate 20, Polysorbate 40, Polysorbate 60, or Polysorbate 80. The number following the ‘polysorbate’ part is related to the type of fatty acid associated with the polyoxyethylene sorbitan part of the molecule. Monolaurate is indicated by 20 (e.g., Polysorbate 20), monopalmitate is indicated by 40 (e.g., Polysorbate 40), monostearate by 60 (e.g., Polysorbate 60), and monooleate by 80 (e.g., Polysorbate 80).

In some embodiments, the stabilizer is Polysorbate 80 (see e.g., Formula XII, below). Polysorbate 80 is also referred to as Polyoxyethylene (20) sorbitan monooleate (number 20 following the ‘polyoxyethylene’ part refers to the total number of oxyethylene —(CH₂CH₂O)—groups found in the molecule); (x)-sorbitan mono-9-octadecenoate poly(oxy-1,2-ethanediyl); MONTANOX 80; ALKEST TW 80; TWEEN 80; or PS 80. Polysorbate 80 is derived from polyethoxylated sorbitan and oleic acid. The hydrophilic groups in this compound are polyethers also known as polyoxyethylene groups, which are polymers of ethylene oxide. In the nomenclature of polysorbates, the numeric designation following polysorbate refers to the lipophilic group, in this case, the oleic acid. The critical micelle concentration of polysorbate 80 in pure water is reported as 0.012 mM.

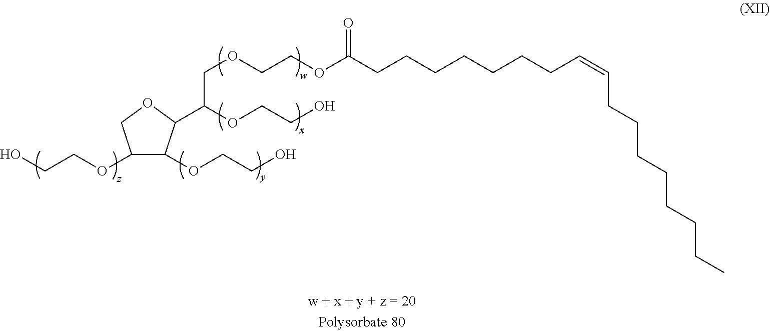

In some embodiments, the stabilizer is a poloxamer. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers are referred to as PLURONIC, KOLLIPHOR, or SYNPERONIC. Because the lengths of the polymer blocks can be customized, many different poloxamers exist that have slightly different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mo, and a 70% polyoxyethylene content). For the PLURONIC and SYNPERONIC names, coding of these copolymers starts with a letter to define its physical form at room temperature (L=liquid, P=paste, F=flake (solid)) followed by two or three digits, The first digit (or two digits in a three-digit number) in the numerical designation, multiplied by 300, indicates the approximate molecular weight of the hydrophobe; and the last digit multiplied by 10 gives the percentage polyoxyethylene content (e.g., L61 indicates a polyoxypropylene molecular mass of 1800 g/mol and a 10% polyoxyethylene content). In the example given, poloxamer 181 (P181)=PLURONIC L61 and SYNPERONIC PE/L 61.

One characteristic of poloxamer solutions is their temperature dependent self-assembling and thermo-gelling behavior. Concentrated aqueous solutions of poloxamers are liquid at low temperature and form a gel at higher temperature in a reversible process. The transitions that occur in these systems depend on the polymer composition. Because of their amphiphilic structures, poloxamers have surfactant properties. Among other things, poloxamers can be used to increase the water solubility of hydrophobic, oily substances or otherwise increase the miscibility of two substances with different hydrophobicities. See e.g., Table 10 for physicochemical properties of exemplary poloxamers.

TABLE 10
Physicochemical properties of PLURONIC copolymers.

| Pluronic ® | | PO | EO | cmc at 25° C. | cmc at 30° C. | cmc at 35° C. |
|---|---|---|---|---|---|---|
| Notation | MW | Units | Units | (% w/v) | (% w/v) | (% w/v) |
| L64 | 2900 | 30 | 26 | n/a | 1.5 | 0.4 |
| P65 | 3400 | 17 | 36 | n/a | 4 | 1 |
| P84 | 4200 | 43 | 38 | 2.6 | 0.6 | 0.15 |
| P85 | 4600 | 40 | 52 | 4 | 0.9 | 0.2 |
| F88 | 11,400 | 39 | 206 | n/a | n/a | 1.7 |
| P103 | 4950 | 60 | 34 | 0.07 | 0.01 | 0.002 |
| P104 | 5900 | 61 | 54 | 0.3 | 0.04 | 0.008 |
| P105 | 6500 | 56 | 74 | 0.3 | 0.025 | 0.005 |
| F108 | 14,600 | 50 | 264 | 4.5 | 0.8 | 0.15 |
| P123 | 5750 | 69 | 38 | 0.03 | 0.005 | 0.001 |
| F127 | 12,600 | 65 | 200 | 0.7 | 0.1 | 0.025 |

MW, molecular weight; PO, propylene oxide; EO, ethylene oxide; cmc, critical micellization concentration; L, liquid; P, paste; F, flake. See e.g., Bodratti and Alexandridis, J Funct Biomater, 2018; 9(1): 11; the content of which is incorporated herein by reference in its entirety.

In some embodiments, the stabilizer is Poloxamer 184 (i.e., PLURONIC L64), Poloxamer 185 (i.e., PLURONIC P65), Poloxamer 234 (i.e., PLURONIC P84), Poloxamer 235 (i.e., PLURONIC P85), Poloxamer 238 (i.e., PLURONIC F88), Poloxamer 333 (i.e., PLURONIC P103), Poloxamer 334 (i.e., PLURONIC P104), Poloxamer 335 (i.e., PLURONIC P105), Poloxamer 338 (i.e., PLURONIC F108), Poloxamer 403 (i.e., PLURONIC P123), or Poloxamer 407 (i.e., PLURONIC F127). In some embodiments, the stabilizer is Poloxamer 184, Poloxamer 185, Poloxamer 234, Poloxamer 235, Poloxamer 238, Poloxamer 333, Poloxamer 334, Poloxamer 335, Poloxamer 338, Poloxamer 403, or Poloxamer 407. In some embodiments, the stabilizer is PLURONIC L64, PLURONIC P65, PLURONIC P84, PLURONIC P85, PLURONIC F88, PLURONIC P103, PLURONIC P104, PLURONIC P105, PLURONIC F108, PLURONIC P123, or PLURONIC F127.

In some embodiments, the stabilizer is poloxamer 188, also referred to as PLURONIC F68 or Polyoxyethylene-polyoxypropylene block copolymer (linear formula: (C₃H₆O·C₂H₄O)ₓ; see e.g., Formula XIII, below). In some embodiments, the pharmaceutical composition comprises Polysorbate 80. In some embodiments, the pharmaceutical composition comprises Poloxamer 188 (i.e., PLURONIC F68). In some embodiments, the pharmaceutical composition comprises Polysorbate 80 and Poloxamer 188 (i.e., PLURONIC F68).

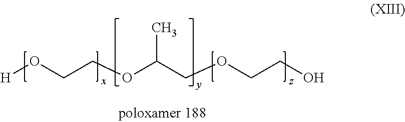

In some embodiments, the stabilizer is polyvinyl alcohol (PVA; see e.g., Formula XIV, below)). PVA is a water-soluble synthetic polymer, with the idealized formula [CH₂CH(OH)]ₙ. PVA can be used as a thickener and emulsion stabilizer. PVA exhibits biocompatibility, a low tendency for protein adhesion, and low toxicity. PVA is prepared by hydrolysis of polyvinyl acetate, or other vinyl ester-derived polymers with formate or chloroacetate groups instead of acetate. The conversion of the polyvinyl esters is can be conducted by base-catalyzed transesterification with ethanol.

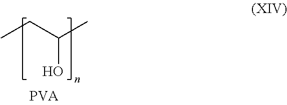

In some embodiments, the pharmaceutical composition comprises at least 0.10% stabilizer by weight. In some embodiments, the pharmaceutical composition comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1.0% or more stabilizer by weight.

In some embodiments, the pharmaceutical composition comprises at least 1.5 mg stabilizer per unit dose. In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, or at least 10 mg stabilizer per unit dose.

In some embodiments, the pharmaceutical composition comprises at least one excipient and at least one stabilizer. In some embodiments, the pharmaceutical composition comprises one excipient and one stabilizer. In some embodiments, the pharmaceutical composition comprises at least two excipients and at least one stabilizer. In some embodiments, the pharmaceutical composition comprises two excipients and one stabilizer. In some embodiments, the pharmaceutical composition comprises leucine and trehalose as excipients and Poloxamer 188 as a stabilizer. In some embodiments, the pharmaceutical composition comprises leucine and trehalose as excipients and Polysorbate 80 as a stabilizer.

### Additives

In some embodiments, the pharmaceutical composition further comprises at least one additive. In some embodiments, the pharmaceutical composition comprises at least 1, 2, 3, 4, 5, 6, 7, 8, 9, 10, or more additives. As described herein, a number of additives can be added to the polymeric particles—these additives can be incorporated by a mixing at a molecule level, a dry blend, a coating onto the particles, or co-administered. In some embodiments, the pharmaceutical composition further comprises at least one of the following: (a) a pore-forming agent; (b) an adhesion agent; (c) a pH-modulating agent; and/or (d) an ester-hydrolysis-inducing agent.

In some embodiments, the pharmaceutical composition further comprises a pore-forming agent. Pore forming agents can decrease the density of the particle (i.e., by forming air spaces or “pores” in the particle) and allow for more rapid water uptake, degradation, and acid production. In some embodiments, the pore-forming agent is selected from the group consisting of: NaCl, sucrose, polyethylene glycol (PEG), and polyvinylpyrrolidone (PVP).

In some embodiments, the pharmaceutical composition further comprises an adhesion agent, which can also be referred to herein as an adhesive compound. Adhesion agents can increase bio-adhesion of the polymer to biological tissue. In some embodiments, the adhesion agent is selected from the group consisting of: sugars, adhesive polymers, and amine-containing compounds. Non-limiting examples of such adhesive sugars include trehalose, mannitol, lactose, or glucose. In some embodiments, the adhesion agent is a tableting agent or a tableting adhesive (e.g., hypromellose or mefenamic acid), which can permit the shaping of a tablet (e.g., for oral administration).

In some embodiments, the pharmaceutical composition further comprises a pH-modulating agent. pH impacting agents can increase or decrease polymer (e.g., polymeric compounds that can produce lactic acid as described herein) degradation. In some embodiments, the pH-modulating agent is a buffer, such as sodium citrate. In some embodiments, the pH-modulating agent is an acid. In some embodiments, the pH-modulating agent is a base (e.g., NaOH); such a base can be a neutralizing agent when creating feedstocks to spray dry for inhaled powders.

In some embodiments, the pharmaceutical composition further comprises an ester-hydrolysis-inducing agent. In some embodiments, the ester-hydrolysis-inducing agent comprises an amine group (e.g., —NH₂). In some embodiments, the ester-hydrolysis-inducing agent is N-hydroxysuccinimide (see e.g., Formula XV below).

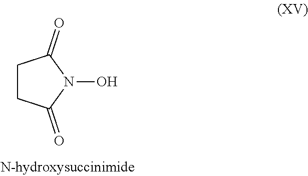

In some embodiments, the pharmaceutical composition further comprises lactic acid. While the polymeric compound (e.g., PLA or PLGA) produces acid upon degradation in the target tissue, a bolus of lactic acid can increase this process or serve as a “loading” level of lactic acid. As used herein, the term “bolus” refers to a single dose of a drug or other medicinal preparation given all at once. In some embodiments, the pharmaceutical composition comprises (a) a lactic acid-producing compound; (b) a pharmaceutically acceptable excipient, stabilizer, or additive; and (c) a bolus dose of lactic acid. In some embodiments, the pharmaceutical composition comprises (a) a non-polymeric lactic acid-producing compound and/or a polymeric lactic acid-producing compound; (b) a pharmaceutically acceptable excipient, stabilizer, or additive; and (c) a bolus dose of lactic acid.

In some embodiments, the pharmaceutical composition comprises a compound that generates acid or produces low pH, in addition to or in place of the lactic acid-producing compound. There are materials other than lactic acid that are organic acids or generate organic acids. These include simple acids such as acetic acid, hydroxy acids such as glycolic acid, multiple functional acids such as citric acid, and aromatic acids such as salicylic acid. Esters, salts, and polymers of these can also be used, including acetylsalicylic acid which can generate acid upon hydrolysis.

In some embodiments, the pharmaceutical composition further comprises at least one acid-generating molecule that can generate acid, wherein the at least one acid-generating molecule is not lactic acid or does not comprise lactic acid. In some embodiments, the acid-generating molecule is selected from the group consisting of: acetic acid, a hydroxy acid, a multiple functional acid, and an aromatic acid, and esters, salts, and polymers thereof. In some embodiments, the acid-generating molecule is selected from the group consisting of: acetic acid, glycolic acid, citric acid, and salicylic acid, and esters, salts, and polymers thereof. In some embodiments, the acid-generating molecule is acetylsalicylic acid. In one aspect, described herein is a pharmaceutical composition comprising: (a) an acid-producing compound (e.g., production of acetylsalicylic acid), and (b) a pharmaceutically acceptable excipient, stabilizer, or additive; wherein the composition is formulated for administration by inhalation, or by oral administration.

In addition to the focus on the generation of therapies based on the production of lactic acid, other acids, or lowering pH, the above constructs can also be used to deliver an additional therapeutic. In some embodiments, the pharmaceutical composition comprises at least one additional therapeutic, e.g., for chronic lung disease. A range of drug classes include anti-inflammatories, antimicrobials, and vasodilators. These drugs can be incorporated using techniques such as microencapsulation, co-formulation, co-administration, or covalently linking with a degradable linkage.

In some embodiments, the pharmaceutical composition comprises an anti-inflammatory. In some embodiments, the anti-inflammatory is selected from the group consisting of: non-steroidal anti-inflammatory drugs (NSAIDs); corticosteroids; glucocorticoids; methotrexate; sulfasalazine; leflunomide; anti-tumor necrosis factor (TNF) medications; cyclophosphamide; pro-resolving lipid mediators; mycophenolate; opiates; and barbiturates.

In some embodiments, the pharmaceutical composition comprises an antimicrobial, an antiviral, and/or an antifungal. In some embodiments, the antimicrobial is selected from the group consisting of: aminoglycosides; ansamycins; beta-lactams; bis-biguanides; carbacephems; carbapenems; cationic polypeptides; cephalosporins; fluoroquinolones; glycopeptides; iron-sequestering glycoproteins; linosamides; lipopeptides; macrolides; monobactams; nitrofurans; oxazolidinones; penicillins; polypeptides; quaternary ammonium compounds; quinolones; silver compounds; sulfonamides; and tetracyclines.

Some exemplary specific antimicrobial agents include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefietazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

Non-limiting examples of antivirals include Abacavir, Acyclovir, Adefovir, Amantadine, Ampligen, Amprenavir, antiretroviral, Arbidol, Atazanavir, Atripla, Cidofovir, Combivir, Darunavir, Delavirdine, Didanosine, Docosanol, Dolutegravir, Ecoliever, Edoxudine, Efavirenz, Emtricitabine, Enfuvirtide, Entecavir, Famciclovir, Fomivirsen, Fosamprenavir, Foscamet, Fosfonet, Fusion inhibitor, Ibacitabine, Idoxuridine, Imiquimod, Imunovir, Indinavir, Inosine, Integrase inhibitor, Interferon, Interferon type I, Interferon type II, Interferon type III, Lamivudine, Lopinavir, Loviride, Maraviroc, Methisazone, Moroxydine, Nelfinavir, Nevirapine, Nexavir, Nitazoxanide, Norvir, Nucleoside analogues, Oseltamivir (Tamiflu), Peginterferon alfa-2a, Penciclovir, Peramivir, Pleconaril, Podophyllotoxin, viral protease inhibitor, Pyramidine, Raltegravir, Reverse transcriptase inhibitor, Ribavirin, Rimantadine, Ritonavir, Saquinavir, Sofosbuvir, Stavudine, Synergistic enhancer (antiretroviral), Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), or Zidovudine.

Non-limiting examples of anti-fungals (also referred to as antimycotics) include polyene antifungals, Amphotericin B, Candicidin, Filipin, Hamycin, Natamycin, Nystatin, Rimocidin, imidazole antifungals, triazole antifungals, thiazole antifungals, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Triazoles, Albaconazole, Efinaconazole, Epoxiconazole, Fluconazole, Isavuconazole, Itraconazole, Posaconazole, Propiconazole, Ravuconazole, Terconazole, Voriconazole, Abafungin, Allylamines, amorolfin, butenafine, naftifine, terbinafine, Echinocandins, Anidulafungin, Caspofungin, Micafungin, Aurones, Benzoic acid, Ciclopirox, Flucytosine, 5-fluorocytosin, Griseofulvin, Haloprogin, Tolnaftate, Undecylenic acid, Triacetin, Crystal violet, Castellani's paint, Orotomide, Miltefosine, Potassium iodide, Coal tar, Copper(II) sulfate, Selenium disulfide, Sodium thiosulfate, Piroctone olamine, Iodoquinol, clioquinol, Acrisorcin, Zinc pyrithione, and Sulfur.

In some embodiments, the pharmaceutical composition comprises at least one bacteriophage. Non-limiting examples of bacteriophage include: Caudovirales such as Myoviridae, Siphoviridae and Podiviridae; Ligamenvirales such as Lipothrixviridae, Rudiviridae, Ampullaviridae, Bicaudaviridae, Clavaviridae, Corticoviridae, Cystoviridae and Fuselloviridae; and other families such as Globuloviridae, Inoviridae, Leviviridae, Microviridae, Plasmaviridae and Techtiviridae.

In some embodiments, the pharmaceutical composition comprises a vasodilator. In some embodiments, the vasodilator is selected from the group consisting of: an angiotensin converting enzyme (ACE) inhibitor; an angiotensin receptor blocker (ARB); a calcium channel blocker (CCB); and a nitric-oxide-producing compound.

In some embodiments, the vasodilator is an ACE inhibitor selected from the group consisting of: benazepril (LOTENSIN); captopril (CAPOTEN); enalapril (VASOTEC, EPANED); fosinopril (MONOPRIL); lisinopril (PRINIVIL, ZESTRIL); moexipril (UNIVASC); perindopril (ACEON); quinapril (ACCUPRIL); ramipril (ALTACE); and trandolapril (MAVIK).

In some embodiments, the vasodilator is an ARB selected from the group consisting of: azilsartan (EDARBI); candesartan (ATACAND); eprosartan (TEVETEN); irbesartan (AVAPRO); telmisartan (MICARDIS); valsartan (DIOVAN); losartan (COZAAR); and olmesartan (BENICAR).

In some embodiments, the vasodilator is a CCB selected from the group consisting of: amlodipine (NORVASC); clevidipine (CLEVIPREX); diltiazem (CARDIZEM); felodipine (CARDENE, CARDENE SR); isradipine; nicardipine; nimodipine; nisoldipine (SULAR); and verapamil (CALAN).

In some embodiments, the vasodilator is a nitric-oxide-producing compound selected from the group consisting of: nitroglycerin (GONITRO, NITROBID, NITROMIST, NITROLINGUAL, NITROSTAT, NITROBID); isosorbide mononitrate (ISMO, MONEKET); isosorbide dinitrate (IMDUR, ISORDIL); hydralazine (APRESOLINE); minoxidil; fenoldopam (CARLOPAM); and nitroprusside (NIPRIDE, NITROPRESS).

In some embodiments, the pharmaceutical composition further comprises a bronchodilator as additional therapeutic. In some embodiments, the bronchodilator is selected from the group consisting of: a short-acting beta-adrenergic bronchodilator (e.g., albuterol, levalbuterol, epinephrine); a long-acting beta-adrenergic bronchodilator (e.g., salmeterol, formoterol); an anticholinergic bronchodilator (e.g., ipratropium bromide, tiotropium bromide); and a xanthine derivative (e.g., theophylline, aminophylline). In some embodiments of any of the aspects, the bronchodilator is albuterol.

In some embodiments, the at least one additional therapeutic is microencapsulated. In some embodiments, the at least one additional therapeutic is microencapsulated within the lactic acid-producing component, e.g., within the matrix of the lactic acid-producing component. In some embodiments, the at least one additional therapeutic is microencapsulated within the matrix of PLA or the matrix of PLGA. In some embodiments, the pharmaceutical composition comprises separate particles or microspheres for the therapeutics and lactic acid, e.g., that are mixed together during administration. In some embodiments, the additional therapeutic is microencapsulated together with the lactic acid-producing compound.

In some embodiments, the at least one additional therapeutic is covalently linked with a biodegradable, degradable, cleavable, or otherwise reversible linker to the lactic acid-producing compound. A cleavable linker means that the linker can be cleaved to release the two parts the linker is holding together. In some embodiments, the cleavable linker is polyethylene glycol. A cleavable linker can be susceptible to cleavage agents, such as, but not limited to, enzymes, pH, redox potential, or the presence of degradative molecules. Examples of such cleavage agents include: redox agents which are selected for particular substrates or which have no substrate specificity, including, e.g., oxidative or reductive enzymes or reductive agents, such as mercaptans, present in cells that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific), proteases, and phosphatases.

In some embodiments, the pharmaceutical composition is co-administered with the at least one additional therapeutic, e.g., for chronic lung disease. In some embodiments, the pharmaceutical composition is administered at the same time as the at least one additional therapeutic, e.g., for chronic lung disease. In some embodiments, the pharmaceutical composition is administered before the at least one additional therapeutic, e.g., for chronic lung disease. In some embodiments, the pharmaceutical composition is administered after at least one additional therapeutic, e.g., for chronic lung disease. In some embodiments, the pharmaceutical composition is administered in alternating times with the at least one additional therapeutic, e.g., for chronic lung disease.

### Formulation

In some embodiments, the pharmaceutical composition is formulated for administration to the lungs. As used herein, the term “formulated for administration to the lungs” means that a composition or formulation is designed or adapted to deliver an active ingredient or agent to the lungs, e.g., by inhalation of small particles or droplets containing the active ingredient. In some embodiments, the pharmaceutical composition is formulated for administration by inhalation. As used herein, the term “formulated for administration by inhalation” means that a composition or formulation is designed or adapted to deliver an active ingredient or agent to bronchopulmonary tissue by inhalation of small particles or droplets containing the active ingredient. As such, a pharmaceutical composition formulated for administration by inhalation generally comprises such particles (e.g., as a powder) or can readily generate such droplets (e.g., via a nebulizer). In some embodiments, the pharmaceutical composition is formulated for delivery to the trachea, the bronchi, the bronchioles, and/or the alveoli. In some embodiments, the pharmaceutical composition is formulated for delivery to the trachea. In some embodiments, the pharmaceutical composition is formulated for delivery to the bronchi. In some embodiments, the pharmaceutical composition is formulated for delivery to the bronchioles. In some embodiments, the pharmaceutical composition is formulated for delivery to the alveoli. In some embodiments, the composition is formulated for delivery to the lungs. In some embodiments, the composition is formulated as a capsule or a tablet for administration by inhalation (e.g., using an inhaler).

In some embodiments, the composition is formulated for delivery by an inhaler. In some embodiments, the composition is formulated for delivery by a metered dose inhaler (MDI). A metered-dose inhaler (MDI) is a device that delivers a specific amount of medication to the lungs, in the form of a short burst of aerosolized medicine that is usually self-administered by the patient via inhalation. In some embodiments, the composition is formulated for delivery by a dry powder inhaler (DPI). A dry-powder inhaler (DPI) is a device that delivers medication to the lungs in the form of a dry powder. In some embodiments, the composition is formulated for delivery by a soft mist inhaler (SMI). A soft mist inhaler (SMI) is a device that delivers medication to the lungs in the form of a mist.

In some embodiments, the pharmaceutical composition is formulated for oral administration. As used herein, the term “formulated for oral administration” means that a composition or formulation is designed or adapted to deliver an active ingredient or agent to gastrointestinal tissue by ingestion of small particles or droplets containing the active ingredient. In some embodiments, the composition formulated for oral administration comprises a tableting agent or a tableting adhesive (e.g., hypromellose or mefenamic acid), which can permit the shaping of a tablet. As such, a pharmaceutical composition formulated for oral administration can be chewed or swallowed whole or otherwise ingestible. In some embodiments, the pharmaceutical composition is formulated for delivery to the colon, mouth, esophagus, stomach, small intestine, duodenum, jejunum, ileum, cecum, ileocecum, appendix, ascending colon, transverse colon, descending colon, sigmoid colon, rectum, or anus. In some embodiments, the composition is formulated as a capsule or a tablet for oral administration.

In some embodiments, the composition is formulated as a bolus dose. In some embodiments, the pharmaceutical composition comprises a bolus dose of lactic acid. In some embodiments, the pharmaceutical composition comprises (a) a non-polymeric lactic acid-producing compound and/or the polymeric lactic acid-producing compound; and (b) a bolus dose of lactic acid. In some embodiments, the pharmaceutical composition comprises (a) a non-polymeric lactic acid-producing compound and/or the polymeric lactic acid-producing compound; and (b) a bolus dose of a non-polymeric compound that can produce lactic acid. In some embodiments, the pharmaceutical composition comprises (a) a non-polymeric lactic acid-producing compound and/or the polymeric lactic acid-producing compound; and (b) a bolus dose of a polymeric compound that can produce lactic acid.

In some embodiments, the pharmaceutical composition comprises at least 7.8 mg of the lactic acid-producing compound per unit dose that is deliverable to a target tissue. In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of the lactic acid-producing compound per unit dose that is deliverable to a target tissue.

In some embodiments, the pharmaceutical composition comprises at least 15 mg of the lactic acid-producing compound per unit dose. In some embodiments, the pharmaceutical composition comprises at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg lactic acid-producing compound per unit dose.

In some embodiments, pharmaceutical composition is formulated as microspheres, e.g., using a method other than spray-drying. Such microspheres can be produced using methods known in the art, including but not limited to solvent extraction, solvent evaporation, and complex coacervation. The four major substeps of microsphere preparation by solvent extraction and evaporation comprise: (i) incorporation of the bioactive compound (e.g., a lactic acid-producing compound as described herein), (ii) formation of the microdroplets, (iii) solvent removal and (iv) harvesting and drying the particles. Complex coacervation is a microencapsulation technique that involves the interaction of oppositely charged polyelectrolytes in aqueous form. In some embodiments, the complex coacervation uses a cationic polymer (e.g., gelatin), and a variety of natural and synthetic anionic water-soluble polymers (e.g., gum arabic) interact with gelatin to form a complex coacervate. Coacervation is a process during which a homogeneous solution of charged macromolecules undergoes liquid-liquid phase separation, giving rise to a polymer-rich dense phase at the bottom and a transparent solution above. See e.g., Freitas et al., Journal of Controlled Release 102(2): 2 313-332 (2005); Timilsena et al., International Journal of Biological Macromolecules 121: 1276-1286 (2019); the contents of each of which are incorporated herein by reference in their entireties.

In some embodiments, the microspheres have a diameter of at least 1 μm to at most 1 mm. In some embodiments, the microspheres have a diameter of at least 1 μm, at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 100 μm, at least 200 μm, at least 300 μm, at least 400 μm, at least 500 μm, at least 600 μm, at least 700 μm, at least 800 μm, at least 900 μm, or at least 1 mm. In some embodiments, the microspheres have a diameter of at most 1 μm, at most 2 μm, at most 3 μm, at most 4 μm, at most 5 μm, at most 6 μm, at most 7 μm, at most 8 μm, at most 9 μm, at most 10 μm, at most 20 μm, at most 30 μm, at most 40 μm, at most 50 μm, at most 60 μm, at most 70 μm, at most 80 μm, at most 90 μm, at most 100 μm, at most 200 μm, at most 300 μm, at most 400 μm, at most 500 μm, at most 600 μm, at most 700 μm, at most 800 μm, at most 900 μm, or at most 1 mm.

### Dried Particles

In some embodiments, the pharmaceutical composition comprises a plurality of dried particles. In some embodiments, the pharmaceutical composition is a dry powder comprising particles with the physical characteristics described in Table 3A, Table 3B, or Table 4.

TABLE 3A
Exemplary Particle Characteristics

| | Range | | |
|---|---|---|---|
| Characteristic Range | | Min | Max |

| MMAD (by impaction) | 3.5 | μm | 7.5 | μm |
|---|---|---|---|---|
| Dispersibility (2/4 KPA | 0.5 | | 1.0 | |
| impaction delivered dose) | | | | |
| Delivered dose (% mass drug | 25.0% | | 80.0% | |
| product/active) | | | | |
| Bulk density | 0.2 | g/cm³ | 0.8 | g/cm³ |
| Tapped density | 0.3 | g/cm³ | 1.0 | g/cm³ |
| Moisture (by Karl Fischer) | 1.0% | w/w | 7.0% | w/w |

TABLE 3B
Exemplary Particle Characteristics

| | Range | | |
|---|---|---|---|
| Characteristic | | Min | Max |

| MMAD (by impaction) | 1.5 | μm | 7.5 | μm |
|---|---|---|---|---|
| Dispersibility (2/4 KPA | 0.5 | | 1.0 | |
| impaction delivered dose) | | | | |
| Delivered dose (% mass drug | 25.0% | | 125% | |
| product/active) | | | | |
| Bulk density | 0.1 | g/cm³ | 0.8 | g/cm³ |
| Tapped density | 0.2 | g/cm³ | 1.0 | g/cm³ |
| Moisture (by Karl Fischer) | 1.0% | w/w | 7.0% | w/w |

TABLE 4
Exemplary Final Particle Characteristics

| Characteristic | Value | Units |
|---|---|---|
| MMAD (by impaction) | 4.8 | μm |
| Dispersibility (2/4 KPA impaction delivered dose) | 0.9 | — |
| Delivered dose (% mass drug product/active) | 52.0 | % |
| Bulk density | 0.5 | g/cm³ |
| Tapped density | 0.6 | g/cm³ |
| Moisture (by Karl Fischer) | 2.3 | % water |

In some embodiments, the dried particles have a Dv50 of at least 0.5 μm. In some embodiments, the dried particles have a Dv50 of at least 2.0 μm. Dv50 is the size at which 50% of the particles are smaller than that size. Dv50 is effectively the median particle diameter, as measured by the technique used. In some embodiments, Dv50 is measured via laser diffraction techniques. Dv50 can apply to aerosolized particle or liquid suspended particles. Dv50 is purely volumetric in nature and does not take into account aerosol properties of the particle. Dv50 can be a useful measure to estimate dispersibility of the powder. Dispersibility refers to how readily a powder aerosolizes at different pressures. A perfectly dispersible powder can aerosolize consistently independent of the pressure applied. In some embodiments, the dried particles have a Dv50 of at least 0.5 um, at least 0.6 um, at least 0.7 um, at least 0.8 um, at least 0.9 um, at least 1 μm, at least 1.5 μm, at least 2 μm, at least 2.5 μm, at least 3 μm, at least 3.5 μm, at least 4 μm, at least 4.5 μm, at least 5 μm, at least 5.5 μm, at least 6 μm, at least 6.5 μm, at least 7 μm, at least 7.5 μm, at least 8 μm, at least 8.5 μm at least 9 μm, at least 9.5 μm, or at least 10 μm.

In some embodiments, the dried particles have a median mass aerodynamic diameter (MMAD) of at least 1.5 μm to at most 7.5 μm. In some embodiments, the dried particles have a median mass aerodynamic diameter (MMAD) of at least 3.5 μm to at most 7.5 μm. In some embodiments, the dried particles have a median mass aerodynamic diameter (MMAD) of at least 2.5 μm to at most 7.5 μm. In some embodiments, the dried particles have a median mass aerodynamic diameter (MMAD) of at least 2.0 μm to at most 7.5 μm. In some embodiments, the dried particles have a median mass aerodynamic diameter (MMAD) of at least 1.0 μm to at most 10 μm. As used herein, the term “MMAD” refers to the value of aerodynamic diameter for which 50% of some quantity in a given aerosol is associated with particles smaller than the MMAD, and 50% of the quantity is associated with particles larger than the MMAD. In some embodiments, the dried particles have a median mass aerodynamic diameter (MMAD) of at least 4.0 μm. In some embodiments, the dried particles have a median mass aerodynamic diameter (MMAD) of at least 4.8 μm. In some embodiments, the dried particles have a median mass aerodynamic diameter (MMAD) of at most 5.0 μm. In some embodiments, the dried particles have a median mass aerodynamic diameter (MMAD) of at least 1 μm, at least 1.5 μm, at least 2 μm, at least 2.5 μm, at least 3 μm, at least 3.5 μm, at least 4 μm, at least 4.5 μm, at least 5 μm, at least 5.5 μm, at least 6 μm, at least 6.5 μm, at least 7 μm, at least 7.5 μm, at least 8 μm, at least 8.5 μm at least 9 μm, at least 9.5 μm, or at least 10 μm. In some embodiments, the dried particles have a median mass aerodynamic diameter (MMAD) of at most 1 μm, at most 1.5 μm, at most 2 μm, at most 2.5 μm, at most 3 μm, at most 3.5 μm, at last 4.0 μm, at most 4.5 μm, at most 5.0 μm, at most 5.5 μm, at most 6.0 μm, at most 6.5 μm, at most 7.0 μm, at most 7.5 μm, at most 8.5 μm at most 9 μm, at most 9.5 μm, or at most 10 μm.

In some embodiments, the dried particles described herein have a fine particle fraction<5.0 m of at least 70%, at least 75%, at least 80%, at least 85%, at least 90%, at least 91%, at least 92%, at least 93%, at least 94%, at least 95%, at least 96%, at least 97%, at least 98%, at least 99%, or more. In some embodiments, the dried particles described herein have a fine particle fraction<3.3 μm of at least 50%, at least 55%, at least 60%, at least 65%, at least 70%, at least 75%, at least 80%, at least 85%, at least 90%, at least 95%, or more.

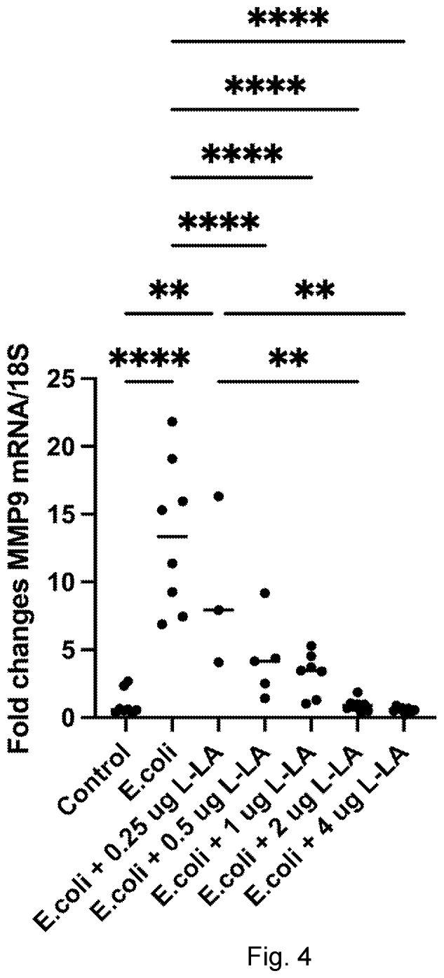

Fine particle fraction (FPF) is a value obtained from cascade impaction testing of a dry powder. In a cascade impaction test, powder is run across stages of different, progressively smaller sizes. The amount of powder deposited at each stage indicates the size cutoff percentages for powder as a whole. Two such methods are Next Generation Impaction (NGI) which flows powder horizontally across stages and Anderson Cascade Impaction (ACI) which flows powder vertically across stages. Typically, two values are provided: the % of the delivered dose smaller than 5.0 μm and the % of the delivered dose smaller than 3.3 um. <5.0 μm is the size cutoff typically regarded to deposit in the central and distal airways and <3.3 μm typically deposited in the distal airways. See e.g., Bianco et al., Respiratory Research volume 22, Article number: 71 (2021) (see e.g., FIG. 4 of Bianco); Darquenne, J Aerosol Med Pulm Drug Deliv. 2012; 25(3): 140-147; next generation impactor description (available on the world at sanyo-si.com/wp-content/uploads/NGI.pdf); the contents of each of which are incorporated herein by reference in their entireties.

In some embodiments, the dried particles have a dispersibility of less than 2.0. In some embodiments, the dried particles have a dispersibility of at least 0.5 to 1.0. As used herein, the term “dispersibility” refers to the ability of a particle to be spatially well-distributed, in terms of size and concentration, in a liquid after a controlled dispersion process. Dispersibility is unit-less. Dispersibility represents a ratio of the particle size measured for the same powder at different air pressures used to send the particles through the system. Specifically, dispersibility is a measurement taken by the Dv50 of a powder sent through a system at a “low” pressure, divided by the Dv50 of a powder sent through a system at a “high” pressure. In some embodiments, the dispersibility of the dried particles is measured as Dv50 measured at a pressure of 2 kPa divided by Dv50 measured at a pressure of 4 kPa (such a measurement can be referred to as “2/4 KPA herein”). Dispersibility can be measured for a delivered (e.g., emitted) dose. Dv50 can be measured either via impaction, laser diffraction (e.g., RODOS or MASTERSIZER), or both. Low pressure systems can have a higher Dv50 when measured using laser diffraction. Bulk and particle density affect dispersibility, as well as “stickiness” and shape of the dried particles, and vice versa. Because dispersibility comprises so many other attributes, dispersibility is a good dimensionless measure of how “well” a particle becomes airborne even if the pressure from air applied to it is not constant. In some embodiments, an “optimal” powder has a dispersibility ratio at 1 or close to 1, meaning its Dv50 does not change across air pressures in an aerodynamic system. As a ratio, the minimum value for dispersibility is zero; dispersibility can be less than 1 or greater than 1.

In some embodiments, the dried particles have a dispersibility of at least 0.9. In some embodiments, the dried particles have a dispersibility of at least 0.50, at least 0.55, at least 0.60, at least 0.65, at least 0.70, at least 0.75, at least 0.80, at least 0.85, at least 0.90, at least 0.91, at least 0.92, at least 0.93, at least 0.94, at least 0.95, at least 0.96, at least 0.97, at least 0.98, at least 0.99, at least 1.0, at least 1.1, at least 1.2, at least 1.3, at least 1.4, at least 1.5, at least 1.6, at least 1.7, at least 1.8, at least 1.9, or 2.0. In some embodiments, the dried particles have a dispersibility of 0.50-0.55, 0.55-0.60, 0.60-0.65, 0.65-0.70, 0.70-0.75, 0.75-0.80, 0.80-0.85, 0.85-0.90, 0.90-0.91, 0.91-0.92, 0.92-0.93, 0.93-0.94, 0.94-0.95, 0.95-0.96, 0.96-0.97, 0.97-0.98, 0.98-0.99, 0.99-1.0, 1.0-1.1, 1.1-1.2, 1.2-1.3, 1.3-1.4, 1.4-1.5, 1.5-1.6, 1.6-1.7, 1.7-1.8, 1.8-1.9, 1.9-2.0, 0.5-0.75, 0.75-1.0, 0.75-1.25, 1.0-1.25, 1.25-1.5, or 0.5-1.5.

In some embodiments, the dried particles have a delivered dose of at least 25.0% to at most 125% of the composition by mass to a target tissue. In some embodiments, the dried particles have a delivered dose of at least 25.0% to at most 80.0% of the composition by mass to a target tissue. As used herein, the term “delivered dose” refers to the percentage of the composition or active ingredient thereof (e.g., lactic acid-producing compound) that is expelled from a delivery device (e.g., inhaler) and/or delivered to a target tissue. In some embodiments, the delivered dose can be greater than 100% when the expected dose in the delivery device (e.g., 1.0 mg) is less than the actual dose in the delivery device (e.g., 1.25 mg; e.g., due to over-filling), and greater than the expected dose (e.g., >1.0 mg; e.g., >100%) but less than the actual dose (e.g., 1.25 mg; e.g., <125%) is delivered to the target tissue. The factors that influence delivered dose are ultimately related to dispersibility, powder density, hygroscopicity, and static charge (e.g., some of these factors affect dispersibility more than other factors). As a non-limiting example, higher density particles can aerosolize and fly better than lower density particles, which can fly unpredictably or be too large. As another non-limiting example, excipients that make the powder less “flowable” or sticky due to being too hygroscopic can make less powder leave the capsule when the inhaler (e.g., a DPI) is actuated. It is anticipated that not all powder from the capsule containing the spray-dried biotherapeutic matrix composition as described herein can make it to the target tissue (e.g., alveoli). A certain portion of the composition can: stay in the capsule, stay in the device, or hit the tongue or esophagus, and thus not be delivered. In some embodiments, the delivered dose refers to the amount of spray-dried biotherapeutic matrix composition as described herein that leaves the device and capsule.

Delivered dose can be measured as the mass % of powder that escapes a delivery system and lands in the target region(s). Emitted dose is a good measurement of what escapes the delivery device in an actual system. Content uniformity of that emitted dose can be measured to ensure that what is emitted from the dosage device is consistent. In some embodiments, the dried particles have a delivered dose of at least 30% of the lactic acid-producing compound by mass to a target tissue. In some embodiments, the dried particles have a delivered dose of at least 52% of the lactic acid-producing compound by mass to a target tissue. In some embodiments, the dried particles have a delivered dose of at least 25%, at least 30%, at least 35%, at least 40%, at least 45%, at least 50%, at least 55%, at least 60%, at least 65%, at least 70%, at least 75%, at least 80% of the lactic acid-producing compound by mass that is expelled from a delivery device (e.g., inhaler) and/or delivered to a target tissue.

In some embodiments, the dried particles have a delivered dose of at least 7.8 mg per unit dose of the lactic acid-producing compound by mass to a target tissue. In some embodiments, the dried particles have a delivered dose of at most 50 mg per unit dose of the lactic acid-producing compound by mass to a target tissue. In some embodiments, the dried particles have a delivered dose of at least 5 mg, at least 10 mg, at least 15 mg, at least 20 mg, at least 25 mg, at least 30 mg, at least 35 mg, at least 40 mg, at least 45 mg, or at least 50 mg per unit dose of the lactic acid-producing compound by mass to a target tissue. In humans, lactic acidosis occurs at levels of 4 mmol/L systemically, while the normal range of lactic acid is 0.5 to 1.5 mmol/L. In some embodiments, the administration of the composition described increases the systemic level of lactic acid by at most 2.5 mmol/L. Based on molecular weight of lactate (89 mg/mmol), the delivered dose of lactic acid is at most 222.5 mg/L or at most 1000 mg of lactic acid overall.

In some embodiments, the particles have a bulk density of at least 0.1 g/cm³ to 0.8 g/cm³. In some embodiments, the particles have a bulk density of at least 0.2 g/cm³ to 0.8 g/cm³. As used herein, “bulk density” is defined as the mass of the many particles of the material divided by the total volume they occupy; the total volume includes particle volume, inter-particle void volume, and internal pore volume. In some embodiments, the particles have a bulk density of at least 0.5 g/cm³. In some embodiments, the particles have a bulk density of at least 0.1 g/cm³, at least 0.2 g/cm³, at least 0.3 g/cm³, at least 0.4 g/cm³, at least 0.5 g/cm³, at least 0.6 g/cm³, at least 0.7 g/cm³, or at least 0.8 g/cm³.

In some embodiments, the particles have a tapped density of at least 0.2 g/cm³ to 1.0 g/cm³. In some embodiments, the particles have a tapped density of at least 0.3 g/cm³ to 1.0 g/cm³. As used herein, “tapped density” refers to the bulk density of the powder after a specified compaction process, e.g., involving vibration of a container containing the powder of dried particles. In some embodiments, the particles have a tapped density of at least 0.6 g/cm³. In some embodiments, the particles have a tapped density of at least 0.1 g/cm³, at least 0.2 g/cm³, at least 0.3 g/cm³, at least 0.4 g/cm³, at least 0.5 g/cm³, at least 0.6 g/cm³, at least 0.7 g/cm³, at least 0.8 g/cm³, at least 0.9 g/cm³, or at least 1.0 g/cm³.

In some embodiments, the particles have a moisture content of at least 1.0% to 7.0% water by weight. As used herein, “moisture content” refers to how much water is in a product, measured as a percentage. The moisture content can influence the physical properties of a substance, including weight, density, viscosity, conductivity, and others. Moisture content can be determined by weight loss upon drying (loss-on drying, LOD). In some embodiments, moisture content is measured using Karl Fischer Titration. Karl Fischer uses coulometric or volumetric titration to determine trace amounts of water in a sample. The elementary reaction responsible for water quantification in the Karl Fischer titration is oxidation of sulfur dioxide with iodine: H₂O+SO₂+I₂→SO₃+2HI. This elementary reaction consumes exactly one molar equivalent of water vs. iodine. Iodine is added to the solution until it is present in excess, marking the end point of the titration, which can be detected by potentiometry. The reaction is run in an alcohol solution containing a base, which consumes the sulfur trioxide and hydroiodic acid produced. The end point is detected most commonly by a bipotentiometric titration method.

In some embodiments, the particles have a moisture content of at least 2.3% water by weight. In some embodiments, the particles have a moisture content of at most 2.3% water by weight. In some embodiments, the particles have a moisture content of at least 1%, at least 1.5%, at least 2%, at least 2.5%, at least 3%, at least 3.5%, at least 4%, at least 4.5%, at least 5%, at least 5.5%, at least 6%, at least 6.5%, at least 7% water by weight. In some embodiments, the particles have a moisture content of at most 1%, at most 1.5%, at most 2%, at most 2.5%, at most 3%, at most 3.5%, at most 4%, at most 4.5%, at most 5%, at most 5.5%, at most 6%, at most 6.5%, at most 7% water by weight.

### Devices

In one aspect, the pharmaceutical composition (e.g., a lactic acid-producing pharmaceutical composition) as described herein is in combination with a delivery device, such as an inhaler. In one aspect, described herein is an inhalation device for bronchopulmonary delivery comprising: (a) an inhaler; and (b) a container containing a pharmaceutical composition (e.g., a lactic acid-producing pharmaceutical composition) as described herein. In some embodiments, the inhaler comprises: (a) a mouthpiece comprising an opening; and (b) means for aerosolizing or dispersing the pharmaceutical composition in the container. In some embodiments, the means for aerosolizing or dispersing include, but are not limited to propellants, pressurized air, a spring, or another chemical or mechanical production of pressure. In some embodiments, the inhaler is a dry powder inhaler (DPI). In some embodiments, the inhaler is a metered dose inhaler (MDI). In some embodiments, the inhaler is a soft mist inhaler (SMI).

In some embodiments, the pharmaceutical composition (e.g., a lactic acid-producing pharmaceutical composition) as described herein is administered as an aerosolized composition. Aerosolization is the process or act of converting some physical substance (e.g., a solid) into the form of particles small and light enough to be carried on the air i.e. into an aerosol. In some embodiments, the pharmaceutical composition (e.g., a lactic acid-producing pharmaceutical composition) as described herein can also be administered directly to the airways in the form of a dry powder. For use as a dry powder, the pharmaceutical composition (e.g., a lactic acid-producing pharmaceutical composition) as described herein can be administered by use of an inhaler. Inhalers are portable, handheld devices that can be available as metered dose inhalers (MDI), dry powder inhalers (DPI), or soft mist inhalers (SMI).

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Especially preferred propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent. The metered dose inhaler allows for the correct dosage of the composition to be delivered to the patient.

A dry powder inhaler (i.e. TURBUHALER (ASTRA AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations of the pharmaceutical composition (e.g., a lactic acid-producing pharmaceutical composition) as described herein thoroughly intermixed with lactose, or other inert powders acceptable for intrabronchial administration. The powder compositions can be administered via an aerosol dispenser or encased in a breakable capsule which may be inserted by the patient into a device that punctures the capsule and blows the powder out in a steady stream suitable for inhalation. The compositions can include propellants, surfactants, and co-solvents and may be filled into conventional aerosol containers that are closed by a suitable metering valve.

A soft mist inhaler (SMI; e.g., Respimat® Soft Mist™ Inhaler) is a multidose, propellant-free, hand-held, liquid inhaler. The aerosol cloud generated by an SMI contains a higher fraction of fine particles than most pressurized metered dose inhalers (pMDIs) and dry powder inhalers (DPIs), and the aerosol spray exits the inhaler more slowly and for a longer duration than with pMDIs. This translates into higher lung drug deposition and lower oropharyngeal deposition, allowing lower nominal doses of delivered drugs without lowering efficacy. In some embodiments, the inhaler formulation does not comprise a propellant. Medication is stored as a solution in the SMI drug cartridge, e.g., an aluminum cylinder containing a double-walled, plastic, collapsible bag that contracts as the solution is used. The SMI solution can be formulated with ethanol or water, and preservatives (e.g., benzalkonium chloride or ethylene diamine tetra-acetic acid (EDTA)). The amount of preservatives in each administration (e.g., puff) can be about 0.44 g for benzalkonium chloride or about 2.2 g for EDTA.

In some embodiments of any of the aspects, the pharmaceutical composition (e.g., a lactic acid-producing pharmaceutical composition) as described herein is administered using a nasal spray or a nebulizer. In some embodiments of any of the aspects, the pharmaceutical composition (e.g., a lactic acid-producing pharmaceutical composition) is formulated as a nasal spray. In one aspect, the pharmaceutical composition (e.g., a lactic acid-producing pharmaceutical composition) as described herein is in combination with a nebulizer. In some embodiments of any of the aspects, the pharmaceutical composition (e.g., a lactic acid-producing pharmaceutical composition) is formulated for delivery by a nebulizer.

A nasal spray typically comprises a saline solution comprising the pharmaceutical composition (e.g., a lactic acid-producing pharmaceutical composition) as described herein, which is sprayed as a mist into the nasal passages using a mechanical spray nozzle. Nebulizers are electric- or battery-powered machines that convert a liquid pharmaceutical composition (e.g., a lactic acid-producing pharmaceutical composition) as described herein into a fine mist that is inhaled into the lungs. The inhaler or nebulizer described herein can further comprise a mouthpiece or facemask. A nasal spray typically administers the pharmaceutical composition (e.g., a lactic acid-producing pharmaceutical composition) as described herein to the upper respiratory tract, whereas the inhaler or nebulizer typically administers the pharmaceutical composition (e.g., a lactic acid-producing pharmaceutical composition) as described herein to the lower respiratory tract.

A pharmaceutical composition (e.g., a lactic acid-producing pharmaceutical composition) as described herein can be administered directly to the airways of a subject in the form of an aerosol or by nebulization. For use as aerosols, a pharmaceutical composition (e.g., a lactic acid-producing pharmaceutical composition) as described herein in solution or suspension can be packaged in a pressurized aerosol container together with suitable propellants, for example, hydrocarbon propellants like propane, butane, or isobutane with conventional adjuvants. The pharmaceutical composition (e.g., a lactic acid-producing pharmaceutical composition) as described herein can also be administered in a non-pressurized form such as in a nebulizer or atomizer.

The term “nebulization” is well known in the art to include reducing liquid to a fine spray. Preferably, by such nebulization small liquid droplets of uniform size are produced from a larger body of liquid in a controlled manner. Nebulization can be achieved by any suitable means therefore, including by using many nebulizers known and marketed today. For example, an AEROMIST pneumatic nebulizer available from Inhalation Plastic, Inc. of Niles, Ill. When the active ingredients are adapted to be administered, either together or individually, via nebulizer(s) they can be in the form of a nebulized aqueous suspension or solution, with or without a suitable pH or tonicity adjustment, either as a unit dose or multi-dose device.

As is well known, any suitable gas can be used to apply pressure during the nebulization, with preferred gases to date being those which are chemically inert to the pharmaceutical composition (e.g., a lactic acid-producing pharmaceutical composition) as described herein. Exemplary gases including, but are not limited to, nitrogen, argon or helium can be used to high advantage.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), the contents of each of which are incorporated herein by reference in their entirety.

### Spray-Drying Methods

Described herein are methods of preparing a spray-dried pharmaceutical compositions, e.g., which comprise a lactic acid-producing compound as described herein. In one aspect, described herein is a method of preparing a spray-dried pharmaceutical composition comprising a lactic acid-producing compound, comprising: (a) preparing a liquid feedstock comprising the lactic acid-producing compound; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the lactic acid-producing compound.

In one aspect, described herein is a method of preparing a spray-dried pharmaceutical composition comprising a lactic acid-producing compound, comprising: (a) obtaining a liquid feedstock comprising the lactic acid-producing compound; (b) introducing droplets of the liquid feedstock through an atomization nozzle into a drying chamber; (c) exposing the liquid feedstock droplets to heated, pressurized gas in the drying chamber to create dried particles; and (d) isolating dried particles of a predetermined range of diameters in a cyclone chamber, wherein the isolated dried particles comprise the lactic acid-producing compound. In some embodiments, the method of preparing a spray-dried pharmaceutical composition comprising a lactic acid-producing compound is performed using at least one of the conditions described in Table 5 or Table 6.

TABLE 5
Exemplary Ranges of Spray Drying Process Conditions

| | Range | | |
|---|---|---|---|
| Condition | | Min | Max |

| | | | | |
|---|---|---|---|---|
| Liquid feedstock solids | 1 | g/L | 100 | g/L |
| concentration | | | | |
| Process gas inlet temperature | 100° | C. | 195° | C. |
| Process gas outlet temperature | 40° | C. | 85° | C. |
| Atomization gas pressure | 10 | psig | 150 | psig |
| Drying gas flow rate | 5 | kg/hr | 150 | kg/hr |
| Feedstock flow rate | 5 | g/min | 1,000 | g/min |

TABLE 6
Exemplary Spray Drying Process Conditions

| Condition | Value | Units |
|---|---|---|
| Process gas inlet temperature | 135 | ° C. |
| Process gas outlet temperature | 60 | ° C. |
| Atomization gas pressure | 20 | psig |
| Drying gas flow rate | 18 | kg/hr |
| Feedstock flow rate | 15 | g/min |

The processing conditions tabulated above in Table 5 and Table 6 provide exemplary ranges across the spray unit operation size. For example, the higher values of gas pressure and flow rate represent conditions closer to a full-scale production unit, and the values on the lower end represent conditions closer to a bench-top or lab scale processing unit.

The drying gas flow rate can be expressed as either kg/hr or L/min. The value of kg/hr can be calculated from the L/min value using the following formula: Mass (kg/hr)=[[density of air (g/L)/volume (L/min)]*(1 kg/1000 g)*(60 min/1 hr)]; the density of air is about 1.225 g/L (e.g., it can vary slightly with temperature, elevation, etc.). For example, 600 L/min is equivalent to 44.1 kg/hr. The value of L/min can be calculated from the kg/hr value using the following formula: Volume (L/min)=[[mass (kg/hr)/density of air (g/L)]*(1000 g/l kg)*(1 hr/60 min)]. For example, 18 kg/hr is equivalent to 245 L/min.

The feedstock flow rate can be expressed as g/min or mL/min; such rates can be converted depending on the density (e.g., g/mL) of the liquid feedstock. The atomization pressure can be expressed as psig, kPa, or other known units for pressure (1 psig=6.89475728 kPa; 345 kPa=−50 psig).

To spray dry compositions comprising lactic acid-producing compound as described herein, target processing conditions are optimized for three characteristics: (1) constituent activity/assay; (2) powder flowability and stability; and (3) aerodynamic size and properties (see e.g., Table 3A-3B and Table 4 for exemplary properties of the spray-dried particles).

In order to protect constituent stability, powder is spray dried at the lowest possible outlet temperature, which is the effective temperature that the dry powder will experience. During drying, a phenomenon known as evaporative cooling keeps the solid constituents relatively cool compared to their heated surroundings. As particle formation completes, the particles heat up to some temperature between room temperature and the temperature of the inlet drying gas. It is this temperature (measured at the outlet of the spray drying chamber) that should be minimized. Pulling in the other direction is the water content of the powder, which can also be minimized to improve long-term stability and physical characteristics. However, higher outlet temperatures for the same formulation typically result from a dryer particle. These two process conditions can be optimized to maximize particle stability and minimize moisture content. In addition, reducing atomization pressure is advantageous to constituent stability; higher pressures put stress on live organisms and sensitive therapeutics, so the atomization pressure used to make droplets should be kept as low as reasonably possible to protect the constituents.

Aerodynamic properties are dominated by the included excipients, particle density, and particle size. The excipients can be chosen to allow for dispersibility (e.g., by addition of an excipient, such as an amino acid, like leucine) and stability (e.g., by addition of a high glass transition temperature, strongly soluble sugar, polyol, or ionic salt; e.g., by the addition of a stabilizer as described herein). Inclusion of agents to increase dispersibility also enhances bulk powder properties like flowability, making the capsule filling process more efficient.

During spray drying, the particle formation is driven by two factors: (1) evaporation rate of the solvent, and (2) solubility of the solutes. A dimensionless number, called the Peclet number (Pe), provides an indication of which of these two phenomena dominate the droplet drying process inside the spray drying chamber, Formula XVI:

$$\begin{matrix}( XVI ) Peclet number calculation & \\ \frac{Droplet evaporation rate}{Diffusion of solutes} = Pe & \end{matrix}$$

When Peclet numbers are high, particles tend to be very low density, large, hollow, and brittle. This is because evaporation of the droplet dominates the movement of solutes to the droplet center (in most systems), leaving a “shell” of solutes. Morphology tends to be spherical. Low Peclet numbers result in small, high-density particles. Solute mass transfer dominates the system as these solids move to the center of the slowly evaporating droplet. Morphology tends to be crumpled.

Often, high process temperatures result in a faster evaporation rate. Lower process temperatures tend to do the opposite, resulting in a dense, crumpled particle, which is the target morphology for our application.

Lower amounts of dissolved solids in the feedstock tend to result in faster diffusion of solutes, which also contributes to a small, dense, crumpled particle. Larger amounts of solids restrict the rate at which solute diffusion can occur but contribute to higher material yield and improved processing.

### Feedstock Preparation

In some embodiments, the method of preparing a spray-dried pharmaceutical composition comprises preparing a liquid feedstock, e g., comprising the lactic acid-producing compound. In some embodiments, the step of preparing the liquid feedstock comprises dissolving a solid feedstock into a solution. In some embodiments, the step of preparing the liquid feedstock comprises dissolving a solid feedstock into an aqueous solution. In some embodiments, the step of preparing the liquid feedstock comprises dissolving a solid feedstock into an organic solution, e.g., for lactic acid-producing compounds (e.g., PLA) that are not soluble in water or aqueous solutions. In some embodiments, the step of preparing the liquid feedstock comprises: (a) dissolving a lactic acid-producing compound into an organic solution; (b) dissolving a solid feedstock into an aqueous solution; (c) combining the resultant solutions from (a) and (b) to produce a liquid feedstock. One example of a solid feedstock for use in preparing an inhaled lactic acid-producing formulation, with the optimal processing condition at lab-scale, can be found below in Table 7.

TABLE 7
Exemplary Solid Feedstock

| Constituent | % w/w |
|---|---|
| Poly(D,L-lactide) polymer | 50.00 |
| Leucine | 10.00 |
| Trehalose | 10.00 |
| Poloxamer 188 (i.e., PLURONIC F68) | 30.00 |

In some embodiments, the solid feedstock comprises: at least 50% lactic acid-producing compound by weight; at least 10% excipient by weight; and at least 1% stabilizer by weight. In some embodiments, the solid feedstock comprises: at least 50% lactic acid-producing compound by weight; at least 20% excipient by weight; and at least 30% stabilizer by weight. In some embodiments, the solid feedstock comprises: at least 50% lactic acid-producing compound by weight. In some embodiments, the solid feedstock comprises: at least 20% excipient by weight. In some embodiments, the solid feedstock comprises: at least 30% stabilizer by weight.

In some embodiments, the solid feedstock comprises: at least 50% lactic acid-producing compound by weight; at least 5% of a first excipient by weight; at least 5% of a second excipient by weight; and at least 1% stabilizer by weight. In some embodiments, the solid feedstock comprises: at least 5% of a first excipient by weight, and at least 5% of a second excipient by weight.

In some embodiments, the solid feedstock comprises: at least 50% lactic acid-producing compound by weight; at least 10% of a first excipient by weight; at least 10% of a second excipient by weight; and at least 30% stabilizer by weight. In some embodiments, the solid feedstock comprises: at least 10% of a first excipient by weight, and at least 10% of a second excipient by weight.

In some embodiments, the solid feedstock comprises at least 20% to at most 80% lactic acid-producing compound by weight. In some embodiments, the solid feedstock comprises: at least 20%, at least 25%, at least 30%, at least 35%, at least 40%, at least 45%, at least 50%, at least 55%, at least 60%, at least 65%, at least 70%, at least 75%, or at least 80%, lactic acid-producing compound by weight.

In some embodiments, the solid feedstock comprises at least 1% to at most 15% excipient by weight. In some embodiments, the solid feedstock comprises at least 5% to at most 15% excipient by weight. In some embodiments, the solid feedstock comprises at least 1%, at least 2%, at least 3%, at least 4%, at least 5%, at least 6%, at least 7%, at least 8%, at least 9%, at least 10%, at least 11%, at least 12%, at least 13%, at least 14%, or at least 15% excipient by weight.

In some embodiments, the solid feedstock comprises at least 0.5%-7.5% of a first excipient by weight, and at least 0.5%-7.5% of a second excipient by weight. In some embodiments, the solid feedstock comprises at least 2.5%-7.5% of a first excipient by weight, and at least 2.5%-7.5% of a second excipient by weight. In some embodiments, the solid feedstock comprises at least 0.5%, at least 1.0%, at least 1.5%, at least 2.0%, at least 2.5%, at least 3.0%, at least 3.5%, at last 4.0%, at least 4.5%, at least 5.0%, at least 5.5%, at least 6.0%, at least 6.5%, at least 7.0%, or at least 7.5% of a first excipient by weight, and at least 0.5%, at least 1.0%, at least 1.5%, at least 2.0%, at least 2.5%, at least 3.0%, at least 3.5%, at last 4.0%, at least 4.5%, at least 5.0%, at least 5.5%, at least 6.0%, at least 6.5%, at least 7.0%, or at least 7.5% of a second excipient by weight. In some embodiments, the solid feedstock comprises at least 0.5%, at least 1.0%, at least 1.5%, at least 2.0%, at least 2.5%, at least 3.0%, at least 3.5%, at last 4.0%, at least 4.5%, at least 5.0%, at least 5.5%, at least 6.0%, at least 6.5%, at least 7.0%, or at least 7.5% of a first excipient by weight, and at least 2.5% of a second excipient by weight. In some embodiments, the solid feedstock comprises at least 2.5% of a first excipient by weight, and at least at least 0.5%, at least 1.0%, at least 1.5%, at least 2.0%, 2.5%, at least 3.0%, at least 3.5%, at last 4.0%, at least 4.5%, at least 5.0%, at least 5.5%, at least 6.0%, at least 6.5%, at least 7.0%, or at least 7.5% of a second excipient by weight.

In some embodiments, the solid feedstock comprises at least 10% to at most 50% stabilizer by weight. In some embodiments, the solid feedstock comprises at least 10%, at least 15%, at least 20%, at least 25%, at least 30%, at least 35%, at least 40%, at least 45%, or at least 50% stabilizer by weight.

As a non-limiting example, the step of preparing the liquid feedstock comprises dissolving 50 mg/mL Poly (D,L-lactide) polymer in acetone (e.g., 2.000 g PLA into 40 mL acetone). The PLA and acetone mixture is added at 1:3-part ratio to water containing 1% Poloxamer 188 (i.e., PLURONIC F68) (e.g., 40 mL PLA & Acetone into ˜120 mL water with 1% Poloxamer 188). An exemplary liquid feedstock formula is provided in Table 8 below; this exemplary liquid feedstock formula is 0.8% w/w solids, or 8 g/L of a 500 g feedstock suspension (i.e., approximately 500 mL, assuming approximately 1 g per 1 mL; as such “0.5 L of the liquid feedstock” can be used interchangeably with “500 g of the liquid feedstock”). Such an exemplary liquid feedstock can be scaled up to a full-scale production unit, e.g., using the same or substantially the same ratios of components.

TABLE 8
Exemplary Liquid Feedstock Formula

| Constituent | Mass (g) | % w/w |
|---|---|---|
| Poly(D,L-lactide) polymer | 2.000 | 0.400 |
| Leucine | 0.400 | 0.080 |
| Trehalose | 0.400 | 0.080 |
| Poloxamer 188 (i.e., | 1.200 | 0.240 |
| PLURONIC F68) | | |
| Acetone | 31.360 | 6.272 |
| Water | 464.640 | 92.93 |

In some embodiments, the liquid feedstock comprises at least 0.1 g/L solid feedstock dissolved in an aqueous solution. In some embodiments, the liquid feedstock comprises at least 0.1 g/L to at most 100 g/L solid feedstock dissolved in an aqueous solution. In some embodiments, the liquid feedstock comprises at least 4 g/L solid feedstock dissolved in an aqueous solution. In some embodiments, the liquid feedstock comprises at least 5 g/L solid feedstock dissolved in an aqueous solution. In some embodiments, the liquid feedstock comprises at least 8 g/L solid feedstock dissolved in an aqueous solution. In some embodiments, the liquid feedstock comprises at least 0.1 g/L, at least 0.2 g/L, at least 0.3 g/L, at least 0.4 g/L, at least 0.5 g/L, at least 0.6 g/L, at least 0.7 g/L, at least 0.8 g/L, at least 0.9 g/L, at least 1 g/L, at least 2 g/L, at least 3 g/L, at least 4 g/L, at least 5 g/L, at least 6 g/L, at least 7 g/L, at least 8 g/L, at least 9 g/L, at least 10 g/L, at least 20 g/L, at least 30 g/L, at least 40 g/L, at least 50 g/L, at least 60 g/L, at least 70 g/L, at least 80 g/L, at least 90 g/L, or at least 100 g/L solid feedstock dissolved in an aqueous solution.

In some embodiments, the liquid feedstock comprises at least 0.01% to at most 10% solid feedstock dissolved in an aqueous solution. In some embodiments, the liquid feedstock comprises at least 0.4% solid feedstock dissolved in an aqueous solution. In some embodiments, the liquid feedstock comprises at least 0.5% solid feedstock dissolved in an aqueous solution. In some embodiments, the liquid feedstock comprises at least 0.8% solid feedstock dissolved in an aqueous solution. In some embodiments, the liquid feedstock comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% or more solid feedstock dissolved in an aqueous solution.

In some embodiments, 0.5 L of the liquid feedstock comprises: (a) at least 2.00 g lactic acid-producing compound; (b) at least 0.80 g excipient; (c) at least 1.2 g stabilizer; (d) at least 31.36 g organic solution; and (e) at least 464.64 g aqueous solution. In some embodiments, 0.5 L of the liquid feedstock comprises: (a) at least 2.00 g lactic acid-producing compound; (b) at least 0.40 g of a first excipient; (c) at least 0.40 g of a second excipient; (d) at least 1.2 g stabilizer; (e) at least 31.36 g organic solution; and (f) at least 464.64 g aqueous solution.

In some embodiments, 0.5 L of the liquid feedstock comprises at least 0.1 g to at most 10 g lactic acid-producing compound. In some embodiments, 0.5 L of the liquid feedstock comprises at least 2 g lactic acid-producing compound. In some embodiments, 0.5 L of the liquid feedstock comprises at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, or at least 10 g lactic acid-producing compound.

In some embodiments, 0.5 L of the liquid feedstock comprises at least 0.1 g to at most 10 g excipient. In some embodiments, 0.5 L of the liquid feedstock comprises at least 0.80 g excipient. In some embodiments, 0.5 L of the liquid feedstock comprises at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, or at least 10 g excipient.

In some embodiments, 0.5 L of the liquid feedstock comprises at least 0.05 g to at most 5 g of a first excipient, and at least 0.05 g to at most 5 g of a second excipient. In some embodiments, 0.5 L of the liquid feedstock comprises at least 0.40 g of a first excipient and at least 0.40 g of a second excipient. In some embodiments, 0.5 L of the liquid feedstock comprises at least 0.05 g, at least 0.06 g, at least 0.07 g, at least 0.08 g, at least 0.09 g, at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, or at least 5 g of a first excipient and at least 0.05 g, at least 0.06 g, at least 0.07 g, at least 0.08 g, at least 0.09 g, at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, or at least 5 g of a second excipient.

In some embodiments, 0.5 L of the liquid feedstock comprises at least 0.05 g, at least 0.06 g, at least 0.07 g, at least 0.08 g, at least 0.09 g, at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, or at least 5 g of a first excipient and at least 0.4 g of a second excipient. In some embodiments, 0.5 L of the liquid feedstock comprises at least 0.4 g of a first excipient and at least 0.05 g, at least 0.06 g, at least 0.07 g, at least 0.08 g, at least 0.09 g, at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, or at least 5 g of a second excipient.

In some embodiments, 0.5 L of the liquid feedstock comprises at least 0.1 g to at most 10 g stabilizer. In some embodiments, 0.5 L of the liquid feedstock comprises at least 1.2 g stabilizer. In some embodiments, 0.5 L of the liquid feedstock comprises at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, or at least 10 g stabilizer.

In some embodiments, 0.5 L of the liquid feedstock comprises at least 10 g to at most 50 g organic solution. In some embodiments, 0.5 L of the liquid feedstock comprises at least 31.36 g organic solution. In some embodiments, 0.5 L of the liquid feedstock comprises at least 10 g, at least 15 g, at least 20 g, at least 25 g, at least 30 g, at least 35 g, at least 40 g, at least 45 g, or at least 50 g organic solution.

In some embodiments, 0.5 L of the liquid feedstock comprises at least 420 g to at most 490 g aqueous solution. In some embodiments, 0.5 L of the liquid feedstock comprises at least 464.64 g aqueous solution. In some embodiments, 0.5 L of the liquid feedstock comprises at least 420 g, at least 425 g, at least 430 g, at least 435 g, at least 440 g, at least 445 g, at least 450 g, at least 455 g, at least 460 g, at least 465 g, at least 470 g, at least 475 g, at least 480 g, at least 485 g, or at least 490 g aqueous solution.

In some embodiments, the liquid feedstock comprises: (a) at least 0.40% lactic acid-producing compound; (b) at least 0.16% excipient; (c) at least 0.24% stabilizer; (d) at least 6.27% organic solution; and (e) at least 92.93% aqueous solution. In some embodiments, the liquid feedstock comprises: (a) at least 0.40% lactic acid-producing compound by weight; (b) at least 0.08% of a first excipient by weight; (c) at least 0.08% of a second excipient by weight; (d) at least 0.24% stabilizer; (e) at least 6.27% organic solution; and (f) at least 92.93% aqueous solution by weight.

In some embodiments, the liquid feedstock comprises at least 0.01% to at most 1.0% lactic acid-producing compound by weight. In some embodiments, the liquid feedstock comprises at least 0.4% lactic acid-producing compound by weight. In some embodiments, the liquid feedstock comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% or more lactic acid-producing compound by weight.

In some embodiments, the liquid feedstock comprises at least 0.01% to at most 10% excipient by weight. In some embodiments, the liquid feedstock comprises at least 0.16% excipient by weight. In some embodiments, the liquid feedstock comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% or more excipient by weight.

In some embodiments, the liquid feedstock comprises at least 0.005% to at most 5% of a first excipient by weight, and at least 0.005% to at most 5% of a second excipient by weight. In some embodiments, the liquid feedstock comprises at least 0.08% of a first excipient by weight, and at least 0.08% of a second excipient by weight. In some embodiments, the liquid feedstock comprises at least 0.005%, at least 0.006%, at least 0.007%, at least 0.008%, at least 0.009%, at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% or more of a first excipient by weight, and at least 0.005%, at least 0.006%, at least 0.007%, at least 0.008%, at least 0.009%, at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% or more of a second excipient by weight.

In some embodiments, the liquid feedstock comprises at least 0.005%, at least 0.006%, at least 0.007%, at least 0.008%, at least 0.009%, at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% or more of a first excipient by weight, and at least 0.08% of a second excipient by weight. In some embodiments, the liquid feedstock comprises at least 0.08% of a first excipient by weight, and at least 0.005%, at least 0.006%, at least 0.007%, at least 0.008%, at least 0.009%, at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, or at least 5% or more of a second excipient by weight.

In some embodiments, the liquid feedstock comprises at least 0.01% to at most 1.0% stabilizer by weight. In some embodiments, the liquid feedstock comprises at least 0.24% stabilizer by weight. In some embodiments, the liquid feedstock comprises at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, or at least 10% or more stabilizer by weight.

In some embodiments, the liquid feedstock comprises at least 1% to at most 5% organic solution by weight. In some embodiments, the liquid feedstock comprises at least 6.27% organic solution by weight. In some embodiments, the liquid feedstock comprises at least 1%, at least 1.5%, at least 2%, at least 2.5%, at least 3%, at least 3.5%, at least 4%, at least 4.5%, or at least 5%, organic solution by weight.

In some embodiments, the liquid feedstock comprises at least 90% to at most 99.9% aqueous solution by weight. In some embodiments, the liquid feedstock comprises at least 92.93% aqueous solution by weight. In some embodiments, the liquid feedstock comprises at least 90%, at least 91%, at least 92%, at least 93%, at least 94%, at least 95%, at least 96%, at least 97%, at least 98%, at least 99%, at least 99.5%, at least 99.9% or more aqueous solution by weight.

In some embodiments, the lactic acid-producing compound is selected from: (a) a polymeric compound that can produce lactic acid; (b) a non-polymeric compound that can produce lactic acid; or (c) lactic acid. In some embodiments, the non-polymeric lactic acid-producing compound is an inorganic salt of lactic acid, an ester of lactic acid, or lactide. In some embodiments, the polymeric lactic acid-producing compound is a polylactic acid (PLA). In some embodiments, the polylactic acid is poly(D,L-lactide) (PDLLA).

In some embodiments, the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts. In some embodiments, the excipient is leucine and/or trehalose.

In some embodiments, the stabilizer is a polysorbate; poloxamer; or polyvinyl alcohol. In some embodiments, the stabilizer is Poloxamer 188 (i.e., PLURONIC F68). In some embodiments, the stabilizer is Polysorbate 80.

In some embodiments, the organic solution is acetone. In some embodiments, the organic solution comprises acetone. In some embodiments, the aqueous solution is water. In some embodiments, the aqueous solution comprises water.

In some embodiments, the liquid feedstock further comprises at least one additional therapeutic. Non-limiting examples of the at least one additional therapeutic include: an anti-inflammatory, an antimicrobial, an antiviral, an antifungal, a vasodilator, or a bronchodilator, as described herein. In some embodiments, the lactic acid-producing compound and at least one additional therapeutic are spray-dried together. In some embodiments, the lactic acid-producing compound and at least one additional therapeutic are spray-dried separately.

### Atomization

Described herein are methods of spray-drying a pharmaceutical composition. Such spray-drying can be performed using spray-drying equipment or technology known in the art. Methods of spray-drying are known in the art and are not limited; see e.g., U.S. Pat. Nos. 7,258,873, 7,378,110, 8,273,374, 8,293,275, 9,238,005, 9,044,497; US Patent Publications US20130022728, US20140086965, US20180027855; the contents of each of which are incorporated herein by reference in their entirety

In one aspect, the method of preparing a spray-dried pharmaceutical composition comprises: (a) introducing droplets of a liquid feedstock as describe herein through an atomization nozzle into a drying chamber; (b) exposing the liquid feedstock droplets to heated, pressurized gas in the drying chamber to create dried particles; and (c) isolating dried particles of a predetermined range of diameters in a cyclone chamber, wherein the isolated dried particles comprise the lactic acid-producing compound.

In some embodiments, the method of preparing a spray-dried pharmaceutical composition comprises introducing droplets of a liquid feedstock as describe herein through an atomization nozzle into a drying chamber. In some embodiments, the atomization nozzle into the drying chamber has a diameter of at least 1.2 mm. Some commercial, full-scale nozzles can have much larger nozzle diameters than 1.2 mm, but spray at much higher atomization pressures (e.g., greater than 150 psig) to re-produce the ideal droplet sizes and dried particle characterizations described herein. In some embodiments, the atomization nozzle into the drying chamber has a diameter of at least 0.5 mm, at least 1 mm, at least 1.5 mm, at least 2 mm, at least 2.5 mm, at least 3 mm, at least 3.5 mm, at least 4 mm, at least 4.5 mm, or at least 5 mm.

In some embodiments, the droplets of liquid feedstock produced by the atomization nozzle into the drying chamber have a diameter of at least 1.2 um. In some embodiments, the droplets of liquid feedstock produced by the atomization nozzle into the drying chamber have a diameter of at least 3.5 um. In some embodiments, the droplets of liquid feedstock produced by the atomization nozzle into the drying chamber have a diameter of at least 0.5 um, at least 1 μm, at least 1.5 um, at least 2 μm, at least 2.5 um, at least 3 μm, at least 3.5 um, at least 4 μm, at least 4.5 um, or at least 5 um.

### Drying Chamber

In some embodiments, the method of preparing a spray-dried pharmaceutical composition comprises exposing the liquid feedstock droplets to heated, pressurized gas in a drying chamber to create dried particles.

In some embodiments, the droplets of liquid feedstock have a flow rate through the drying chamber of at least 5 g/min. In some embodiments, the droplets of liquid feedstock have a flow rate through the drying chamber of at least 15 g/min. In some embodiments, the droplets of liquid feedstock have a flow rate through the drying chamber of at most 1000 g/min. In some embodiments, the droplets of liquid feedstock have a flow rate through the drying chamber of at least 5 g/min, at least 10 g/min, at least 15 g/min, at least 20 g/min, at least 25 g/min, at least 30 g/min, at least 35 g/min, at least 40 g/min, at least 45 g/min, at least 50 g/min, at least 60 g/min, at least 70 g/min, at least 80 g/min, at least 90 g/min, at least 100 g/min, at least 200 g/min, at least 300 g/min, at least 400 g/min, at least 500 g/min, at least 600 g/min, at least 700 g/min, at least 800 g/min, at least 900 g/min, or at least 1000 g/min.

In some embodiments, the heated, pressurized gas is filtered before being inlet into the drying chamber. In some embodiments, the filter is a high efficiency particulate air (HEPA) filter. In some embodiments, the filter removes contaminants of at least 0.3 um. In some embodiments, the filter removes contaminants of at least 0.01 um, at least 0.02 um, at least 0.03 um, at least 0.04 um, at least 0.05 um, at least 0.06 um, at least 0.07 um, at least 0.08 um, at least 0.09 um, at least 0.1 um, at least 0.2 um, at least 0.3 um, at least 0.4 um, at least 0.5 um, at least 0.6 um, at least 0.7 um, at least 0.8 um, at least 0.9 um, or at least 1 um

In some embodiments, the heated, pressurized gas is heated before being inlet into the drying chamber. In some embodiments, the heated, pressurized gas is inlet into the drying chamber at a temperature of at least 100° C. In some embodiments, the heated, pressurized gas is inlet into the drying chamber at a temperature of at least 135° C. In some embodiments, the heated, pressurized gas is inlet into the drying chamber at a temperature of at most 195° C. In some embodiments, the heated, pressurized gas is inlet into the drying chamber at a temperature of at least 100° C., at least 105° C., at least 110° C., at least 115° C., at least 120° C., at least 125° C., at least 130° C., at least 135° C., at least 140° C., at least 145° C., at least 150° C., at least 155° C., at least 160° C., at least 165° C., at least 170° C., at least 175° C., at least 180° C., at least 185° C., at least 190° C., or at least 195° C.

In some embodiments, the heated, pressurized gas is outlet from the drying chamber at a temperature of at least 40° C. In some embodiments, the heated, pressurized gas is outlet from the drying chamber at a temperature of at least 60° C. In some embodiments, the heated, pressurized gas is outlet from the drying chamber at a temperature of at most 85° C. In some embodiments, the heated, pressurized gas is outlet from the drying chamber at a temperature of at least 40° C., at least 45° C., at least 50° C., at least 55° C., at least 60° C., at least 65° C., at least 70° C., at least 75° C., at least 80° C., or at least 85° C.

In some embodiments, the heated, pressurized gas is pressurized before being inlet into the drying chamber. In some embodiments, the heated, pressurized gas in the drying chamber has an atomization gas pressure of at least 10 pounds per square inch gauge (psig). In some embodiments, the heated, pressurized gas in the drying chamber has an atomization gas pressure of at least 20 pounds per square inch gauge (psig). In some embodiments, the heated, pressurized gas in the drying chamber has an atomization gas pressure of at most 150 pounds per square inch gauge (psig). In some embodiments, the heated, pressurized gas in the drying chamber has an atomization gas pressure of at least 10 psig, at least 20 psig, at least 30 psig, at least 40 psig, at least 50 psig, at least 60 psig, at least 70 psig, at least 80 psig, at least 90 psig, at least 100 psig, at least 110 psig, at least 120 psig, at least 130 psig, at least 140 psig, at least 150 psig.

In some embodiments, the heated, pressurized gas has a flow rate through the drying chamber of at least 5 kg/hr. In some embodiments, the heated, pressurized gas has a flow rate through the drying chamber of at least 18 kg/hr. In some embodiments, the heated, pressurized gas has a flow rate through the drying chamber of at most 150 kg/hr. In some embodiments, the heated, pressurized gas has a flow rate through the drying chamber of at least 5 kg/hr, at least 6 kg/hr, at least 7 kg/hr, at least 8 kg/hr, at least 9 kg/hr, at least 10 kg/hr, at least 11 kg/hr, at least 12 kg/hr, at least 13 kg/hr, at least 14 kg/hr, at least 15 kg/hr, at least 16 kg/hr, at least 17 kg/hr, at least 18 kg/hr, at least 19 kg/hr, at least 20 kg/hr, at least 30 kg/hr, at least 40 kg/hr, at least 50 kg/hr, at least 60 kg/hr, at least 70 kg/hr, at least 80 kg/hr, at least 90 kg/hr, at least 100 kg/hr, at least 110 kg/hr, at least 120 kg/hr, at least 130 kg/hr, at least 140 kg/hr, at least 150 kg/hr.

Spray drying can be performed in batches, depending on the stability of the feedstock and availability to run the dryer for a certain amount of time. In some embodiments, spray-drying batches last an 8-hour day, but can be longer depending on the manufacturer. After this time, the equipment is cleaned and prepared for another run with “fresh” equipment. This batch time depends on the size of the drying chamber, which determines the scale at which one can spray dry (assuming the drying gas flow rate and temperature can be increased to match). In some embodiments, the step of exposing the liquid feedstock droplets to heated, pressurized gas in the drying chamber takes at most 8 hours, e.g., 8 hours per batch cycle.

In some embodiments, the step of exposing the liquid feedstock droplets to heated, pressurized gas in the drying chamber takes at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, or at least 24 hours. In some embodiments, the step of exposing the liquid feedstock droplets to heated, pressurized gas in the drying chamber takes at most 1 hour, at most 2 hours, at most 3 hours, at most 4 hours, at most 5 hours, at most 6 hours, at most 7 hours, at most 8 hours, at most 9 hours, at most 10 hours, at most 11 hours, at most 12 hours, at most 18 hours, or at most 24 hours.

In order to establish a gram-per-minute powder production rate, this can be converted from the solids flowrate into the drying chamber via two values: 1) the mass concentration in the feedstock, and 2) the mass flowrate of feedstock into the drying chamber.

### Cyclone Chamber

In some embodiments, the method of preparing a spray-dried pharmaceutical composition comprises isolating dried particles of a predetermined range of diameters in a cyclone chamber. In some embodiments, the heated, pressurized gas is outlet through the cyclone chamber.

In some embodiments, the step of isolating dried particles of a predetermined range of diameters in a cyclone chamber occurs continuously. Powders that settle in the cyclone chamber and collection vessel at the bottom of the cyclone chamber do so continuously. Isolating does not necessarily take a set amount of time

In some embodiments, the dried particles isolated in the cyclone chamber have a median mass aerodynamic diameter (MMAD) of at least 1.5 μm to at most 7.5 μm. In some embodiments, the dried particles isolated in the cyclone chamber have a median mass aerodynamic diameter (MMAD) of at least 3.5 μm to at most 7.5 μm. In some embodiments, the dried particles isolated in the cyclone chamber have a median mass aerodynamic diameter (MMAD) of at least 4.0 μm. In some embodiments, the dried particles isolated in the cyclone chamber have a median mass aerodynamic diameter (MMAD) of at least 4.8 μm. In some embodiments, the dried particles isolated in the cyclone chamber have a median mass aerodynamic diameter (MMAD) of at most 5 μm. In some embodiments, the dried particles isolated in the cyclone chamber have a median mass aerodynamic diameter (MMAD) of at least 3.5 μm, at last 4.0 μm, at least 4.5 μm, at least 5.0 μm, at least 5.5 μm, at least 6.0 μm, at least 6.5 μm, at least 7.0 μm, or at least 7.5 μm.

In embodiments such as those involving inhalation formulations, especially for administration using a DPI, the spray-dried particles can be capsulated (i.e., made into capsules) following their isolation from the cyclone chamber of the spray drier. In embodiments such as those involving oral formulations, the spray-dried particles can be tableted following their isolation from the cyclone chamber of the spray drier. Tableting methods are known in the art and can comprises steps of weighing, milling, mixing, granulation, drying, compaction, coating and/or packaging.

### Administration

In some embodiments, the methods described herein relate to treating a subject having or diagnosed as having a bronchopulmonary disease. In some embodiments, the subject has or is diagnosed as having a chronic bronchopulmonary disease. In some embodiments, the subject has or is diagnosed as having an infectious bronchopulmonary disease. Subjects having a chronic bronchopulmonary disease can be identified by a physician using current methods of diagnosing a chronic bronchopulmonary disease. Symptoms and/or complications of a chronic bronchopulmonary disease which characterize these conditions and aid in diagnosis are well known in the art and include but are not limited to, difficulty breathing; shortness of breath, especially during physical activities; wheezing; chest tightness; a chronic cough that can produce mucus (sputum); frequent respiratory infections; lack of energy; unintended weight loss; and/or swelling in ankles, feet or legs. Tests that may aid in a diagnosis of, e.g. a chronic bronchopulmonary disease include, but are not limited to, lung (pulmonary) function tests; chest x-rays; a lung CT scan; arterial blood gas analysis; and/or laboratory tests (e.g., genetic tests for a gene signature associated with a specific chronic bronchopulmonary disease). A family history of a chronic bronchopulmonary disease, or exposure to risk factors for a chronic bronchopulmonary disease (e.g. long-term exposure to irritating gases or particulate matter; e.g., exposure to cigarette smoke) can also aid in determining if a subject is likely to have a chronic bronchopulmonary disease or in making a diagnosis of a chronic bronchopulmonary disease.

Subjects having an infectious bronchopulmonary disease can be identified by a physician using current methods of diagnosing an infectious bronchopulmonary disease. Symptoms and/or complications of an infectious bronchopulmonary disease which characterize these conditions and aid in diagnosis are well known in the art and include but are not limited to, chest pain when breathing or coughing; confusion or changes in mental awareness (e.g., in adults age 65 and older); cough, which can produce phlegm; fatigue; fever, sweating and shaking chills; lower than normal body temperature (e.g., in adults older than age 65 or people with weak immune systems); nausea, vomiting or diarrhea; and/or shortness of breath. Tests that may aid in a diagnosis of, e.g. an infectious bronchopulmonary disease include, but are not limited to, blood tests; blood cultures; oximetry; arterial blood gases; bronchoscopy; transtracheal mucus culture; lung biopsy; thoracentesis; and/or computed tomography (CT) scan. A family history of infectious bronchopulmonary disease, or exposure to risk factors for infectious bronchopulmonary disease (e.g. long-term exposure to irritating gases or particulate matter; exposure to cigarette smoke; autoimmunity; allergies; asthma; pregnancy) can also aid in determining if a subject is likely to have an infectious bronchopulmonary disease or in making a diagnosis of an infectious bronchopulmonary disease.

In some embodiments, the subject has been diagnosed with or is at risk of developing a bronchopulmonary disease. In some embodiments, the subject has been diagnosed with or is at risk of developing a chronic or infectious bronchopulmonary disease. In some embodiments, the chronic bronchopulmonary disease is selected from the group consisting of: bronchopulmonary dysplasia (BPD), chronic obstructive pulmonary disease (COPD), bronchiectasis, non-cystic fibrosis (CF) bronchiectasis, cystic fibrosis (CF), acute respiratory distress syndrome (ARDS), idiopathic pulmonary fibrosis (IPF), interstitial lung disease (ILD), pleural effusion (PE), pulmonary hypertension (PAH), silicosis, and lung cancer. In some embodiments, the lung cancer is small cell lung cancer (SCLC) or non-small cell lung cancer (NSCLC).

In some embodiments, the infectious bronchopulmonary disease is caused by or associated with an infectious agent selected from: adenovirus; coronavirus (e.g., common cold viruses; Severe Acute Respiratory Syndrome corona virus 1 (SARS-CoV-1); SARS-CoV-2; Middle East Respiratory Syndrome (MERS) CoV); influenza virus (e.g., flu); parainfluenza virus; parvovirus B19 (e.g., parvovirus B19; fifth disease); respiratory syncytial virus (RSV); rhinovirus (e.g., common cold); enterovirus (e.g., EV-D68); measles virus; rubella virus; varicella virus (e.g., chicken pox); Corynebacterium diphtheriae (e.g., diphtheria); Haemophilus influenzae (e.g., type b); Legionella pneumophila (e.g., Legionnaire's disease); Bordetella pertussis (pertussis); Mycobacterium tuberculosis (e.g., tuberculosis); Streptococcus species (e.g., Streptococcus pneumoniae, Streptococcus pyogenes; e.g., pneumonia); Pseudomonas species (e.g., Pseudomonas aeruginosa; e.g., lung infections, chronic recurrent respiratory infections); Escherichia coli (e.g., community-acquired pneumonia); Aspergillus species (e.g., Aspergillus fumigatus, Aspergillus flavus; Aspergillosis); Cryptococcus species (e.g., Cryptococcus neoformans, Cryptococcus gattii; pulmonary cryptococcosis); and Pneumocystis species (e.g., Pneumocystis jirovecii; pneumocystosis); see e.g., Dasaraju and Liu, “Chapter 93: Infections of the Respiratory System,” Medical Microbiology. 4th edition (1996) for non-limiting examples of infectious bronchopulmonary diseases.

In one aspect, described herein is a method of delivering a spray-dried pharmaceutical composition comprising a lactic acid-producing compound to a subject, comprising: (a) obtaining an inhalation device for bronchopulmonary delivery comprising: (i) an inhaler; and (ii) a container containing a spray-dried pharmaceutical composition comprising a lactic acid-producing compound; (b) activating the inhaler to cause aerosolization or dispersal of the spray-dried pharmaceutical composition; and (c) inhaling the aerosolized or dispersed spray-dried pharmaceutical composition.

In one aspect, described herein is a method of delivering a spray-dried pharmaceutical composition (e.g., comprising a lactic acid-producing compound) to a subject, comprising: (a) obtaining an inhalation device for bronchopulmonary delivery comprising: (i) an inhaler; and (ii) a container containing a spray-dried pharmaceutical composition as described herein; (b) activating the inhaler to cause aerosolization or dispersal of the spray-dried pharmaceutical composition; and (c) inhaling the aerosolized or dispersed spray-dried pharmaceutical composition.

In one aspect, described herein is a method of delivering a spray-dried pharmaceutical composition comprising a lactic acid-producing compound to a subject, comprising: (a) obtaining an inhalation device as described herein; (b) activating the inhaler to cause aerosolization or dispersal of the spray-dried pharmaceutical composition; and (c) inhaling the aerosolized or dispersed spray-dried pharmaceutical composition.

In some embodiments, the inhaler is a dry powder inhaler (DPI). In some embodiments, the inhaler is a metered dose inhaler (MDI). In some embodiments, the inhaler is a soft mist inhaler (SMI). In some embodiments, the inhaler comprises: (a) a mouthpiece comprising an opening; and (b) means for aerosolizing or dispersing the spray-dried pharmaceutical composition in the container. In some embodiments, the inhaler has an inspiration flow rate of at least 15 L/min. In some embodiments, the inhaler has an inspiration flow rate of at least 15 L/min to at most 60 L/min. In some embodiments, the inhaler has an inspiration flow rate of at least 15 L/min, at least 20 L/min, at least 25 L/min, at least 30 L/min, at least 35 L/min, at least 40 L/min, at least 45 L/min, at least 50 L/min, at least 55 L/min, or at least 60 L/min.

In some embodiments, at least 25% to at most 125% of the spray-dried pharmaceutical composition by mass is delivered to a target bronchopulmonary tissue. In some embodiments, at least 25% to at most 80% of the spray-dried pharmaceutical composition by mass is delivered to a target bronchopulmonary tissue. In some embodiments, at least 30.0% of the spray-dried pharmaceutical composition by mass is delivered to a target bronchopulmonary tissue. In some embodiments, at least 52.0% of the spray-dried pharmaceutical composition by mass is delivered to a target bronchopulmonary tissue. In some embodiments, at least 25%, at least 30%, at least 35%, at least 40%, at least 45%, at least 50%, at least 55%, at least 60%, at least 65%, at least 70%, at least 75%, or at least 80% of the spray-dried pharmaceutical composition by mass is delivered to a target bronchopulmonary tissue.

In some embodiments, at least 25% to at most 125.0% of the spray-dried pharmaceutical composition by mass is expelled from the inhalation device (e.g., inhaler). In some embodiments, at least 25% to at most 80.0% of the spray-dried pharmaceutical composition by mass is expelled from the inhalation device (e.g., inhaler). In some embodiments, at least 52.0% of the spray-dried pharmaceutical composition by mass is expelled from the inhalation device (e.g., inhaler). In some embodiments, at least 25%, at least 30%, at least 35%, at least 40%, at least 45%, at least 50%, at least 55%, at least 60%, at least 65%, at least 70%, at least 75%, or at least 80% of the spray-dried pharmaceutical composition by mass is expelled from the inhalation device (e.g., inhaler).

In some embodiments, the target bronchopulmonary tissue is the lungs, the trachea, the bronchi, the bronchioles, or the alveoli. In some embodiments, the target bronchopulmonary tissue is the lungs. In some embodiments, the target bronchopulmonary tissue is the trachea. In some embodiments, the target bronchopulmonary tissue is the bronchi. In some embodiments, the target bronchopulmonary tissue is the bronchioles. In some embodiments, the target bronchopulmonary tissue is alveoli.

In some embodiments, the dried particles have a median mass aerodynamic diameter (MMAD) of at least 0.5 μm to at most 10 μm, which can influence which bronchopulmonary regions the particles can reach. Particles from about 0.5 um to about 2.0 um are deposited in the alveoli and small airways, such as bronchioles, which is therapeutically useful. Particles from about 2.0 um to about 10.0 um are deposited in the tracheobronchial region of the airways, which is therapeutically useful. Particles less than 0.5 um are essentially exhaled, which is not therapeutically useful. Particles larger than about 10.0 um are retained in the oropharyngeal region and larynx due to impaction, which is not therapeutically useful. See e.g., Thakur et al., Chapter 22: Mucoadhesive drug delivery systems in respiratory diseases, in Targeting Chronic Inflammatory Lung Diseases Using Advanced Drug Delivery Systems, 2020, pp. 475-491; the content of which is incorporated herein by reference in its entirety.

In some embodiments, the spray-dried pharmaceutical composition is delivered from the bronchopulmonary tissue to a distal tissue site (i.e., non-pulmonary tissue) via the cardiovascular system or lymphatic system. In some embodiments, the distal tissue site (i.e., non-pulmonary tissue) is in the gastrointestinal system, cardiovascular system, lymphatic system, musculoskeletal system, nervous system, urinary system, reproductive system, endocrine system, or integumentary system. In some embodiments, the distal tissue site (i.e., non-pulmonary tissue) is connective tissue, epithelial tissue, muscle tissue, or nervous tissue.

The compositions and methods described herein can be administered to a subject having or diagnosed as having a bronchopulmonary disease, e.g., a chronic bronchopulmonary disease. In some embodiments, the methods described herein comprise administering an effective amount of a composition described herein, e.g., a spray-dried pharmaceutical composition (e.g., comprising a lactic acid-producing compound) as described herein to a subject in order to alleviate a symptom of a chronic bronchopulmonary disease. As used herein, “alleviating a symptom of a chronic bronchopulmonary disease” is ameliorating any condition or symptom associated with the chronic bronchopulmonary disease. As compared with an equivalent untreated control, such reduction is by at least 5%, 10%, 20%, 40%, 50%, 60%, 80%, 90%, 95%, 99% or more as measured by any standard technique.

Oral ingestion and inhalation are two delivery options contemplated herein for modulating the lung microenvironment. The gut-lung axis allows for crosstalk between bacterial metabolic byproducts in the gut and the microenvironment of the lung. Metabolites broken down by commensal bacteria in the gut travel across the intestinal barrier into systemic circulation, thereby affecting inflammation and immune response in the lungs. There are two main methods of creating an orally ingested drug product—tableting and encapsulation. Tableting requires significant granulation and drying followed by high-pressure tableting and coating. The high pressures make tableting live biotherapeutics challenging, as stability rates are low. However, for more robust molecules, tableting can be an effective method for precisely formulating powder blends, which are often spray dried themselves. Encapsulation is an effective way to create an orally ingestible powder blend. Components are blended together with excipients that allow for a flowable powder and a well-sealed capsule for storage and consistent ingestion. Both dosing formats can be coated to protect from certain pH levels in the stomach to allow for more effective absorption and less dose loss and potential side-effects. See e.g., Anand and Mande 2018, Front Microbiol 9:2147; Enaud et al. 2020, Front Cell Infect Microbiol 10:9; Encenarro 2018; the contents of each of which are incorporated herein by reference in their entireties.

Compared with other routes of administration, inhalation offers a number of unique benefits (see e.g., Borghardt et al. 2018, the content of which is incorporated herein by reference in its entirety). Inhalation delivers bacterial byproducts or extracts directly to the lung tissue, resulting in high pulmonary drug concentrations and low systemic drug concentrations, and is associated with minimal systemic side effects. Inhaled formulations containing lactic acid could be created through a number of processing routes, including spray drying to create a dry powder inhaled formulation, compounding to create a nebulizable formulation, or compounding and pressurizing in metered dose inhalers. Each of these routes of administration offer benefits. Dry powder formulations are composed of engineered particles with specific size distributions, allowing for more precisely tuned lung deposition, aerodynamic properties, long-term stability, and relative ease of dosing in patients with normal inhalation capacity. Nebulized solutions containing active pharmaceutical ingredients are well-suited for infant or adolescent lungs, as well as patients with weaker lung capacity. These solutions face stability challenges and have size limitations that can prevent aerosolization during dosing. Metered dose inhalers are common in the market for bronchodilators and inhaled corticosteroids, but typically require a pressurized container and a chemical propellant to work effectively. Some of these propellants have unknown side-effects, and sustained pressure on a drug-containing solution may have stability challenges, especially when formulating with more sensitive biotherapeutics.

The term “effective amount” as used herein refers to the amount of a spray-dried pharmaceutical composition (e.g., comprising a lactic acid-producing compound) as described herein needed to alleviate at least one or more symptom of the disease or disorder, and relates to a sufficient amount of pharmacological composition to provide the desired effect. The term “therapeutically effective amount” therefore refers to an amount of a spray-dried pharmaceutical composition (e.g., comprising a lactic acid-producing compound) as described herein that is sufficient to provide a particular anti-bronchopulmonary disease effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population).

The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a lactic acid-producing compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for pH or apoptosis, among others. The dosage can be determined by a physician and adjusted, as necessary, to suit observed effects of the treatment.

Pharmaceutical compositions comprising a lactic acid-producing compound as described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the lactic acid-producing compound, and can be prepared by methods of pharmacy well known to those skilled in the art. See generally, Remington: The Science and Practice of Pharmacy, 21st Ed., Lippincott, Williams, and Wilkins, Philadelphia PA. (2005).

In some embodiments of any of the aspects, the pharmaceutical composition (e.g., comprising a lactic acid-producing compound) described herein is administered as a monotherapy, e.g., another treatment for the chronic bronchopulmonary disease is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gammalI and calicheamicin omegaIl (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003). In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In some embodiments, the spray-dried biotherapeutic matrix composition is co-administered with the at least one additional therapeutic treatment or therapeutic intervention for a chronic bronchopulmonary disorder.

In some embodiments, the chronic bronchopulmonary disorder is chronic obstructive pulmonary disease (COPD), and the at least one additional therapeutic treatment or therapeutic intervention is: smoking cessation; bronchodilators; inhaled steroids; oral steroids; roflumilast (DALIRESP; a phosphodiesterase-4 inhibitor); theophylline (ELIXOPHYLLIN, THEO-24, THEOCHRON); antibiotics; oxygen therapy; pulmonary rehabilitation (e.g., education, breathing exercises, nutrition advice, and/or counseling); in-home noninvasive ventilation therapy (e.g., bilevel positive airway pressure (BiPAP)); and/or surgery (e.g., lung volume reduction surgery; bullectomy; or lung transplant).

In some embodiments, the chronic bronchopulmonary disorder is lung cancer, and the at least one additional therapeutic treatment or therapeutic intervention is: surgery (e.g., wedge resection; segmental resection; lobectomy; or pneumonectomy); radiation therapy; chemotherapy; stereotactic body radiotherapy; gene-targeted drug therapy; or immunotherapy.

In some embodiments, the chronic bronchopulmonary disorder is asthma, and the at least one additional therapeutic treatment or therapeutic intervention is: bronchodilators; inhaled corticosteroids (e.g., fluticasone propionate (FLOVENT HFA, FLOVENT DISKUS, XHANCE), budesonide (PULMICORT FLEXHALER, PULMICORT RESPULES, RHINOCORT), ciclesonide (ALVESCO), beclomethasone (QVAR REDIHALER), mometasone (ASMANEX HFA, ASMANEX TWISTHALER) and fluticasone furoate (ARNUITY ELLIPTA); leukotriene modifiers. (e.g., montelukast (SINGULAIR), zafirlukast (ACCOLATE) and zileuton (ZYFLO); combination treatments of a long-acting beta agonist and a corticosteroid (e.g., fluticasone-salmeterol (ADVAIR HFA, AIRDUO DIGIHALER), budesonide-formoterol (SYMBICORT), formoterol-mometasone (DULERA) and fluticasone furoate-vilanterol (BREO ELLIPTA); theophylline (ELIXOPHYLLIN, THEO-24, THEOCHRON); short-acting beta agonists (e.g., albuterol (PROAIR HFA, VENTOLIN HFA) and levalbuterol (XOPENEX, XOPENEX HFA); anticholinergic agents (e.g., ipratropium (ATROVENT HFA) and tiotropium (SPIRIVA, SPIRIVA RESPIMAT); oral and intravenous corticosteroids (e.g., prednisone (PREDNISONE INTENSOL, RAYOS) and methylprednisolone (MEDROL, DEPO-MEDROL, SOLU-MEDROL); allergy shots (e.g., injections with a specific allergen to gradually reduce immune system reaction); allergy biologics (e.g., omalizumab (XOLAIR), mepolizumab (NUCALA), dupilumab (DUPIXENT), reslizumab (CINQAIR) or benralizumab (FASENRA)); or bronchial thermoplasty.

In some embodiments, the chronic bronchopulmonary disorder is bronchiectasis, and the at least one additional therapeutic treatment or therapeutic intervention is: antibiotics; macrolides; mucus thinning medication (e.g., guaifenesin (MUCINEX)); airway clearance devices (e.g., positive expiratory pressure (PEP) devices; percussive, wearable devices such as percussive vests); or chest physical therapy (e.g., chest clapping).

In some embodiments, the chronic bronchopulmonary disorder is emphysema, and the at least one additional therapeutic treatment or therapeutic intervention is: bronchodilators; inhaled steroids; antibiotics; pulmonary rehabilitation (e.g., breathing exercises); nutrition therapy; supplemental oxygen; smoking cessation; or surgery (e.g., lung volume reduction surgery; lung transplant).

In some embodiments, the chronic bronchopulmonary disorder is cystic fibrosis (CF), and the at least one additional therapeutic treatment or therapeutic intervention is: cystic fibrosis transmembrane conductance regulator (CFTR) modulators (e.g., a combination medication containing elexacaftor, ivacaftor and tezacaftor (TRIKAFTA); a combination medication containing tezacaftor and ivacaftor (SYMDEKO); a combination medication containing lumacaftor and ivacaftor (ORKAMBI); ivacaftor (KALYDECO); airway clearance devices (e.g., positive expiratory pressure (PEP) devices; percussive, wearable devices such as percussive vests); chest physical therapy (e.g., chest clapping); pulmonary rehabilitation (e.g., breathing exercises); nasal and sinus surgery; oxygen therapy; noninvasive ventilation; feeding tube; bowel surgery; lung transplant; or liver transplant.

In some embodiments, the chronic bronchopulmonary disorder is bronchopulmonary dysplasia (BPD), and the at least one additional therapeutic treatment or therapeutic intervention is: diuretics (e.g., to decrease the amount of fluid in and around the alveoli); bronchodilators; corticosteroids; cardiac medications; or respiratory syncytial virus (RSV) immunization to prevent or reduce respiratory tract infections.

In some embodiments, the chronic bronchopulmonary disorder is acute respiratory disease syndrome (ARDS), and the at least one additional therapeutic treatment or therapeutic intervention is: Supplemental oxygen; Mechanical ventilation; intravenous fluids; smoking cessation; antibiotics; analgesics; anti-inflammatories; blood thinners; gastric reflux drugs (e.g., esomeprazole (NEXIUM)); or sedation.

In some embodiments, the chronic bronchopulmonary disorder is idiopathic pulmonary fibrosis (IPF), and the at least one additional therapeutic treatment or therapeutic intervention is: anti-fibrotics (e.g., Nintedanib (OFEV) or pirfenidone (ESBRIET)); corticosteroids (e.g., prednisone); immunosuppressive drugs (e.g., azathioprine (IMURAN), cyclophosphamide (CYTOXAN), or mycophenolate mofetil (CELLCEPT)); oral or spray antioxidant (e.g., N-acetylcysteine, NAC (MUCOMYST)); oxygen therapy; or pulmonary rehabilitation (e.g., breathing exercises).

In some embodiments, the chronic bronchopulmonary disorder is interstitial lung disease (ILD), and the at least one additional therapeutic treatment or therapeutic intervention is: a corticosteroid (e.g., prednisone); an immunosuppressant; a medication that slows the progression of idiopathic pulmonary fibrosis, such as pirfenidone (ESBRIET) or nintedanib (OFEV); H-2-receptor antagonists or proton pump inhibitors such as lansoprazole (PREVACID 24HR), omeprazole (Prilosec OTC) or pantoprazole (PROTONIX), e.g., for symptoms of gastroesophageal reflux disease (GERD) that affect the majority of people with idiopathic pulmonary fibrosis; oxygen therapy; pulmonary rehabilitation; and/or surgery, such as lung transplantation.

In some embodiments, the chronic bronchopulmonary disorder is pleural effusion (PE), and the at least one additional therapeutic treatment or therapeutic intervention is: a diuretic; chemotherapy; radiation therapy; therapeutic thoracentesis; tube thoracostomy; a sclerosing agent (e.g., talc, doxycycline, and tetracycline); and/or surgery, such as video-assisted thoracoscopic surgery (VATS) or thoracotomy.

In some embodiments, the chronic bronchopulmonary disorder is pulmonary hypertension (PAH), and the at least one additional therapeutic treatment or therapeutic intervention is: a vasodilator; a guanylate cyclase (GSC) stimulator such as riociguat (ADEMPAS); an endothelin receptor antagonist such as bosentan (TRACLEER), macitentan (OPSUMIT) or ambrisentan (LETAIRIS); phosphodiesterase 5 (PDE5) inhibitors such as sildenafil (REVATIO, VIAGRA) or tadalafil (ADCIRCA, CIALIS, ALYQ); a calcium channel blocker such as amlodipine (NORVASC), diltiazem (CARDIZEM, TIAZAC) or nifedipine (PROCARDIA); an anticoagulant such as warfarin (JANTOVEN); digoxin (LANOXIN); diuretics; oxygen therapy; and/or surgery, such as atrial septostomy or lung or heart-lung transplant.

In some embodiments, the chronic bronchopulmonary disorder is silicosis, and the at least one additional therapeutic treatment or therapeutic intervention is: a bronchodilator; cessation of smoking; supplemental oxygen; pulmonary rehabilitation; and/or surgery, such as lung transplantation.

In some embodiments, the at least one additional therapeutic treatment or therapeutic intervention for the infectious bronchopulmonary disorder is an antiviral. an antibiotic, or an antifungal e.g., non-limiting examples of which are listed above.

In certain embodiments, an effective dose of a composition comprising a lactic acid-producing compound as described herein can be administered to a patient once. In certain embodiments, an effective dose of a composition comprising a lactic acid-producing compound can be administered to a patient repeatedly. For systemic administration, subjects can be administered a therapeutic amount of a composition comprising a lactic acid-producing compound, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of the chronic bronchopulmonary disease by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the lactic acid-producing compound. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a lactic acid-producing compound can be administered over a period of time, such as over a 5 minute, 10 minute, 15 minute, 20 minute, or 25 minute period.

The dosage ranges for the administration of a pharmaceutical composition (e.g., comprising a lactic acid-producing compound), according to the methods described herein depend upon, for example, the form of the lactic acid-producing compound, its potency, and the extent to which symptoms, markers, or indicators of a condition described herein are desired to be reduced. The dosage should not be so large as to cause adverse side effects, such as acidosis or tissue damage. Generally, the dosage will vary with the age, condition, and sex of the patient and can be determined by one of skill in the art. The dosage can also be adjusted by the individual physician in the event of any complication.

The efficacy of the pharmaceutical composition (e.g., comprising a lactic acid-producing compound) in, e.g. the treatment of a condition described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms; or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of bronchopulmonary dysplasia (BPD), chronic obstructive pulmonary disease (COPD), bronchiectasis, non-cystic fibrosis (CF) bronchiectasis, cystic fibrosis (CF), acute respiratory distress syndrome (ARDS), idiopathic pulmonary fibrosis (IPF), or lung cancer. When using an experimental animal model, efficacy of treatment is evidenced when a statistically significant change in a marker is observed.

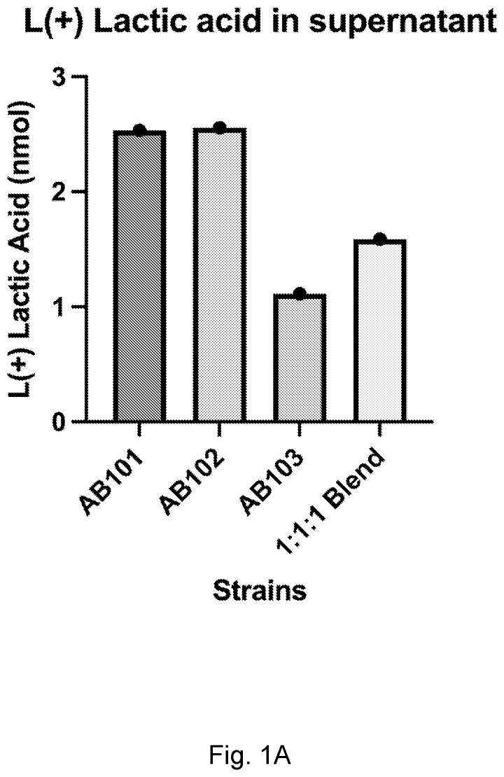

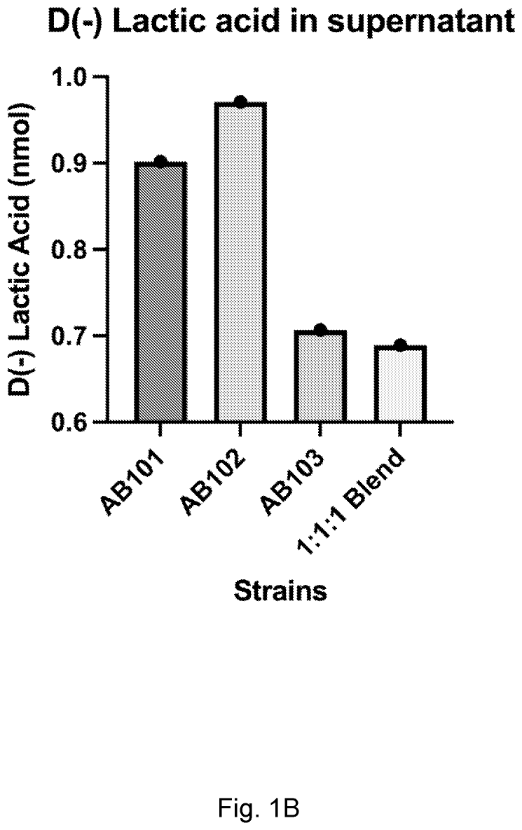

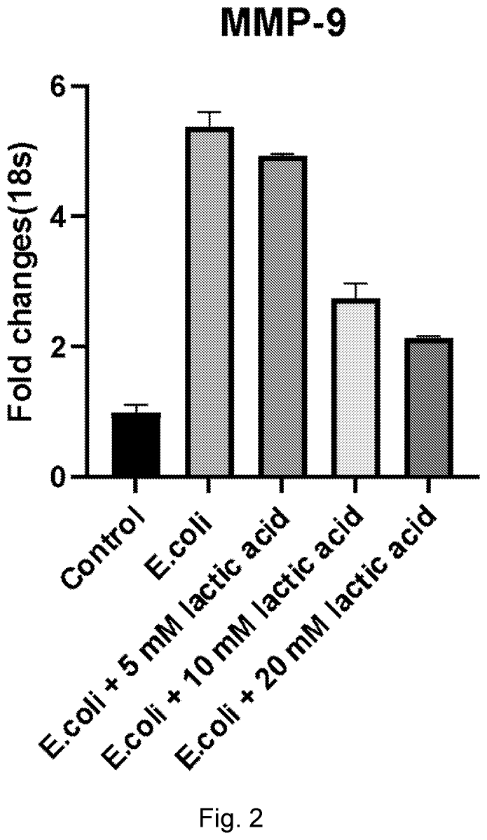

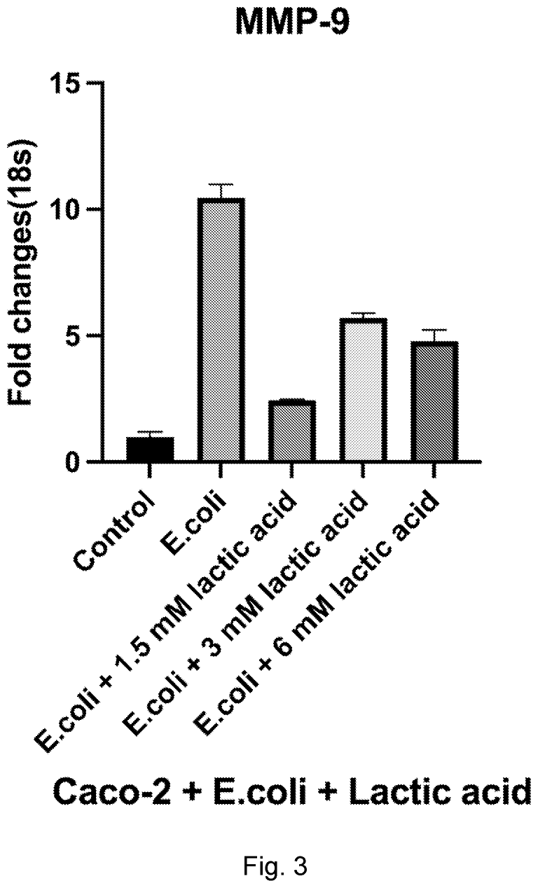

In vitro and animal model assays are provided herein which allow the assessment of a given dose of a pharmaceutical composition (e.g., comprising a lactic acid-producing compound) (see e.g., FIG. 1-7, Example 1). By way of non-limiting example, the effects of a dose of a pharmaceutical composition (e.g., comprising a lactic acid-producing compound) can be assessed using: a dysbiosis model of lung epithelial cells using human primary epithelial (HBE) cells (see e.g., FIG. 2, 5), human intestinal epithelial cells (IECs) from the Caco-2 cell line (see e.g., FIG. 3), A549 non-small cell lung cancer (NSCLC) adenocarcinoma cells (see e.g., FIG. 4), or a murine model of dysbiosis. Non-limiting examples of protocols for such assays are provided in Example 1.

### Treatment Methods

The compositions described herein can be administered to a subject in need thereof, for instance for the treatment of a bronchopulmonary disease, including, but not limited to a chronic bronchopulmonary disease. In some embodiments, the method of treatment can comprise first diagnosing a subject or patient who can benefit from treatment by a composition described herein. In some embodiments, the method further comprises administering to the patient a composition as described herein.

In one aspect, described herein is a method of treating a subject in need thereof comprising administering through inhalation an effective dose of a pharmaceutical composition comprising a lactic acid-producing compound. In one aspect, described herein is a method of treating a subject in need thereof comprising administering through inhalation an effective dose of a pharmaceutical composition as described herein.

In some embodiments, the lactic acid-producing compound reduces neutrophilic inflammation in a target tissue. In some embodiments, the lactic acid-producing compound reduces neutrophilic inflammation in a target tissue by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more.

In some embodiments, the target tissue is a target bronchopulmonary tissue. In some embodiments, the target bronchopulmonary tissue is the lungs, the trachea, the bronchi, the bronchioles, and/or the alveoli. In some embodiments, the target tissue is a distal tissue site from the lungs delivered via the cardiovascular system or lymphatic system.

In some embodiments, the subject has been diagnosed with or is at risk of developing a chronic bronchopulmonary disease. In some embodiments, the chronic bronchopulmonary disease is selected from the group consisting of: asthma, bronchopulmonary dysplasia (BPD), chronic obstructive pulmonary disease (COPD), bronchiectasis, non-cystic fibrosis (CF) bronchiectasis, cystic fibrosis (CF), acute respiratory distress syndrome (ARDS), idiopathic pulmonary fibrosis (IPF), interstitial lung disease (ILD), pleural effusion (PE), pulmonary hypertension (PAH), silicosis, and lung cancer. In some embodiments, the lung cancer is small cell lung cancer (SCLC) or non-small cell lung cancer (NSCLC).

In some embodiments, the chronic bronchopulmonary disease is bronchopulmonary dysplasia (BPD). In some embodiments, the chronic bronchopulmonary disease is chronic obstructive pulmonary disease (COPD). In some embodiments, the chronic bronchopulmonary disease is bronchiectasis. In some embodiments, the chronic bronchopulmonary disease is non-cystic fibrosis (CF) bronchiectasis. In some embodiments, the chronic bronchopulmonary disease is cystic fibrosis (CF). In some embodiments, the chronic bronchopulmonary disease is acute respiratory distress syndrome (ARDS). In some embodiments, the chronic bronchopulmonary disease is idiopathic pulmonary fibrosis (IPF). In some embodiments, the chronic bronchopulmonary disease is interstitial lung disease (ILD). In some embodiments, the chronic bronchopulmonary disease is pleural effusion (PE). In some embodiments, the chronic bronchopulmonary disease is pulmonary hypertension (PAH). In some embodiments, the chronic bronchopulmonary disease is silicosis. In some embodiments, the chronic bronchopulmonary disease is lung cancer. In some embodiments, the chronic bronchopulmonary disease is small cell lung cancer (SCLC). In some embodiments, the chronic bronchopulmonary disease is non-small cell lung cancer (NSCLC). In some embodiments, the chronic bronchopulmonary disease is asthma. In some embodiments, the chronic bronchopulmonary disease is emphysema.

In some embodiments, the subject has been diagnosed with or is at risk of developing an infectious bronchopulmonary disease. In some embodiments, the infectious bronchopulmonary disease is caused by or associated with an infectious agent selected from: adenovirus; coronavirus (e.g., common cold viruses; Severe Acute Respiratory Syndrome corona virus 1 (SARS-CoV-1); SARS-CoV-2; Middle East Respiratory Syndrome (MERS) CoV); influenza virus (e.g., flu); parainfluenza virus; parvovirus B19 (e.g., parvovirus B19; fifth disease); respiratory syncytial virus (RSV); rhinovirus (e.g., common cold); enterovirus (e.g., EV-D68); measles virus; rubella virus; varicella virus (e.g., chicken pox); Corynebacterium diphtheriae (e.g., diphtheria); Haemophilus influenzae (e.g., type b); Legionella pneumophila (e.g., Legionnaire's disease); Bordetella pertussis (pertussis); Mycobacterium tuberculosis (e.g., tuberculosis); Streptococcus species (e.g., Streptococcus pneumoniae, Streptococcus pyogenes; e.g., pneumonia); Pseudomonas species (e.g., Pseudomonas aeruginosa; e.g., lung infections, chronic recurrent respiratory infections); Escherichia coli (e.g., community-acquired pneumonia); Aspergillus species (e.g., Aspergillus fumigatus, Aspergillus flavus; Aspergillosis); Cryptococcus species (e.g., Cryptococcus neoformans, Cryptococcus gattii; pulmonary cryptococcosis); and Pneumocystis species (e.g., Pneumocystis jirovecii; pneumocystosis).

In some embodiments, the effective dose of the pharmaceutical composition is at least 1 mg lactic acid-producing compound per unit dose. In some embodiments, the effective dose of the pharmaceutical composition is at least 5 mg lactic acid-producing compound per unit dose. In some embodiments, the effective dose of the pharmaceutical composition is at least 7.8 mg lactic acid-producing compound per unit dose. In some embodiments, the effective dose of the pharmaceutical composition is at most 50 mg lactic acid-producing compound per unit dose. In some embodiments, the effective dose of the pharmaceutical composition is at least 5 mg, at least 10 mg, at least 15 mg, at least 20 mg, at least 25 mg, at least 30 mg, at least 35 mg, at least 40 mg, at least 45 mg, or at least 50 mg lactic acid-producing compound per unit dose.

In some embodiments, the pharmaceutical composition formulated for oral administration is co-administered with at least one additional therapeutic for a chronic or infectious bronchopulmonary disorder. In some embodiments, the at least one additional therapeutic is an anti-inflammatory, an antimicrobial, an antiviral, an antifungal, a vasodilator, or a bronchodilator, as described further herein. In some embodiments, the pharmaceutical composition comprises lactic acid-producing compound and the at least one additional therapeutic in the same composition or unit dosage. As a non-limiting example, the lactic acid-producing compound and the at least one additional therapeutic can be spray-dried together and formulated for administration together, e.g., in a single capsule. In some embodiments, the lactic acid-producing compound and the at least one additional therapeutic are each spray-dried separately and formulated for administration, e.g., in the same or different capsules. In some embodiments, the lactic acid-producing compound and the at least one additional therapeutic co-administered using a combination delivery device, such as an inhaler device that administers multiple different formulations at once.

In one aspect, described herein is a method of treating a subject in need thereof comprising orally administering an effective dose of a pharmaceutical composition comprising a lactic acid-producing compound. In one aspect, described herein is a method of treating a subject in need thereof comprising orally administering an effective dose of a pharmaceutical composition as described herein. In some embodiments, the target tissue (e.g., of the orally formulated pharmaceutical composition as described herein) is a bronchopulmonary target tissue. In some embodiments, the subject (e.g., who is being treated with the orally formulated pharmaceutical composition as described herein) has been diagnosed with or is at risk of developing a bronchopulmonary disorder, including but not limited to a chronic or infectious bronchopulmonary disorder.

In some embodiments, the pharmaceutical composition comprising a lactic acid-producing compound is administered in conjunction with a standard of care for the chronic or infectious bronchopulmonary disease, as known to a person of skill in the art. As used herein, the term “standard of care” refers to the level at which the average, prudent provider in a given community would practice, e.g., in treating a given indication.

As a non-limiting example, the COPD standard of care changes based on GOLD stage classification of severity, but can include a variety of therapies combining anti-inflammatory action (w.g., inhaled steroids) and symptom relief (e.g., long-acting beta-agonist (LABA), long-acting muscarinic antagonist (LAMA)). Pulmonary fibrosis standard of care is typically at least one of two antifibrotics: nintedanib and pirfenidone. Bronchopulmonary dysplasia standard of care includes a variety of treatments based on the risk profile of the patient including supplemental oxygen, surfactants, and bronchodilators. See e.g., Safka et al., Chronic Obstr Pulm Dis. 2017; 4(1): 45-55 (see e.g., Table 1 of Safka); 2022 Gold Reports, available on the world wide web at goldcopd.org/2022-gold-reports (see e.g., Page 49-57; Page 49 Table 3.3; Page 51 Table 3.4; Page 53 Table 3.5 of the Gold Reports); Maher et al. “Respiratory Research volume 20, Article number: 205 (2019). Non-limiting examples of additional therapeutic treatment or therapeutic intervention for specific chronic or infectious bronchopulmonary diseases are described further herein.

### Unit Dosage Forms

In one aspect, described herein is a unit dosage form comprising a pharmaceutical composition as described herein. In some embodiments, a single unit dosage is one capsule comprising a pharmaceutical composition as described herein. In some embodiments, a single unit dosage is a plurality of (e.g., 1, 2, 3, 4, 5, or more) capsules, each comprising a pharmaceutical composition as described herein. In some embodiments, the unit dosage is administered (e.g., inhaled) during one inhalation (e.g., in less than 10, 20, or 30 seconds). In some embodiments, the unit dosage is administered (e.g., inhaled) over a period of time (e.g., greater than 30, 60, or 120 seconds) and/or administered (e.g., inhaled) with multiple usages of an administering inhaler.

In some embodiments, the unit dosage comprising a pharmaceutical composition as described herein results in about 7.8 mg of PLA being dosed directly to the lungs via inhalation per dose. Intravenous doses of PLA used previously for encapsulation of active drugs were safely used in the range of 40-165 mg per dose; see e.g., Jain et al. (2016) Adv Drug Deliv Rev 107:213-227; the content of which is incorporated by reference herein in its entirety. Once the final dry powder is produced, it can be filled into capsules or tablets for ingestion. The dry powder blend itself can go through a number of process steps that would result in a uniform blend and strong solid dosage form. Such a unit dosage can be formulated for administration by inhalation or by oral administration (see e.g., Tables 1, 2, 11).

In one aspect, described herein is a unit dosage form comprising at least 1.0 mg to at most 100 mg of a pharmaceutical composition comprising a lactic acid-producing compound. In one aspect, described herein is a unit dosage form comprising at least 5.0 mg to at most 100 mg of a pharmaceutical composition comprising a lactic acid-producing compound. In some embodiments, the unit dosage form comprises at least 15.0 mg lactic acid-producing compound per unit dose. In some embodiments, the unit dosage form comprises at least 50.0 mg lactic acid-producing compound per unit dose. In some embodiments, the unit dosage form comprises at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg lactic acid-producing compound per unit dose.

In one aspect, described herein is a unit dosage form comprising at least 1.0 mg to at most 100 mg of a pharmaceutical composition as described herein. In one aspect, described herein is a unit dosage form comprising at least 5.0 mg to at most 100 mg of a pharmaceutical composition as described herein. In one aspect, described herein is a unit dosage form comprising at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a pharmaceutical composition as described herein.

In one aspect, described herein is a unit dosage form comprising at least 1.0 mg to at most 100 mg of the spray-dried pharmaceutical composition prepared by the methods as described herein. In one aspect, described herein is a unit dosage form comprising at least 5.0 mg to at most 100 mg of the spray-dried pharmaceutical composition prepared by the methods as described herein. In one aspect, described herein is a unit dosage form comprising at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of the spray-dried pharmaceutical composition prepared by the methods as described herein.

In one aspect, described herein is a unit dosage form comprising at least 15.0 mg to at most 100 mg of a pharmaceutical composition comprising at least 15 mg lactic acid-producing compound per unit dose. In one aspect, described herein is a unit dosage form comprising at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a pharmaceutical composition comprising at least 15 mg lactic acid-producing compound per unit dose.

In some embodiments, the dosage is at least 1.0 mg pharmaceutical composition. In some embodiments, the dosage is at least 5.0 mg pharmaceutical composition. In some embodiments, the dosage is at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg pharmaceutical composition.

In some embodiments, the dosage comprises at least 1.0 mg lactic acid-producing compound per unit dose that is deliverable to a target tissue. In some embodiments, the dosage comprises at least 5.0 mg lactic acid-producing compound per unit dose that is deliverable to a target tissue. In some embodiments, the dosage comprises at least 7.8 mg lactic acid-producing compound per unit dose that is deliverable to a target tissue. In some embodiments, the dosage comprises at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg lactic acid-producing compound per unit dose that is deliverable to a target tissue.

In some embodiments, the dosage comprises at least 1.0 mg lactic acid-producing compound per unit dose that is expelled by from a delivery device (e.g., inhaler). In some embodiments, the dosage comprises at least 5.0 mg lactic acid-producing compound per unit dose that is expelled by from a delivery device (e.g., inhaler). In some embodiments, the dosage comprises at least 7.8 mg lactic acid-producing compound per unit dose that is expelled by from a delivery device (e.g., inhaler). In some embodiments, the dosage comprises at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg lactic acid-producing compound per unit dose that is expelled by from a delivery device (e.g., inhaler).

In some embodiments, the dosage comprises at least 1 mg lactic acid-producing compound per unit dose. In some embodiments, the dosage comprises at least 5 mg lactic acid-producing compound per unit dose. In some embodiments, the dosage comprises at least 15 mg lactic acid-producing compound per unit dose. In some embodiments, the dosage comprises at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg lactic acid-producing compound per unit dose. In some embodiments, the dosage comprises at most 1.0 mg, at most 2.0 mg, at most 3.0 mg, at most 4.0 mg, at most 5.0 mg, at most 10 mg, at most 15 mg, at most 20 mg, at most 25 mg, at most 30 mg, at most 35 mg, at most 40 mg, at most 45 mg, at most 50 mg, at most 55 mg, at most 60 mg, at most 65 mg, at most 70 mg, at most 75 mg, at most 80 mg, at most 85 mg, at most 90 mg, at most 95 mg, or at most 100 mg lactic acid-producing compound per unit dose.

In some embodiments, the dosage comprises at least 14.25 mg excipient(s) per unit dose. In some embodiments, the dosage comprises at least 144 mg excipient(s) per unit dose. In some embodiments, the dosage comprises at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg or more excipient(s) per unit dose. In some embodiments, the dosage comprises at most 1.0 mg, at most 2.0 mg, at most 3.0 mg, at most 4.0 mg, at most 5.0 mg, at most 10 mg, at most 15 mg, at most 20 mg, at most 25 mg, at most 30 mg, at most 35 mg, at most 40 mg, at most 45 mg, at most 50 mg, at most 55 mg, at most 60 mg, at most 65 mg, at most 70 mg, at most 75 mg, at most 80 mg, at most 85 mg, at most 90 mg, at most 95 mg, at most 100 mg, at most 110 mg, at most 120 mg, at most 130 mg, at most 140 mg, at most 150 mg, at most 160 mg, at most 170 mg, at most 180 mg, at most 190 mg, or at most 200 mg excipient(s) per unit dose.

In some embodiments, the dosage comprises at least 7.125 mg of a first excipient and at least 7.125 mg of a second excipient per unit dose. In some embodiments, the dosage comprises at least 72 mg of a first excipient and at least 72 mg of a second excipient per unit dose. In some embodiments, the dosage comprises at least 0.5 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a first excipient per unit dose, and at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a second excipient per unit dose.

In some embodiments, the dosage comprises at least 7.125 mg of a first excipient, and at least 0.5 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a second excipient per unit dose. In some embodiments, the dosage comprises at least 0.5 mg, at least 1.0 mg, at least 2.0 mg, at least 3.0 mg, at least 4.0 mg, at least 5.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, or at least 100 mg of a first excipient per unit dose, and at least 7.125 mg of a second excipient per unit dose.

In some embodiments, the dosage comprises at least 0.45 mg stabilizer per unit dose. In some embodiments, the dosage comprises at least 3.0 mg stabilizer per unit dose. In some embodiments, the dosage comprises at least 0.1 mg, at least 0.2 mg, at least 0.3 mg, at least 0.4 mg, at least 0.5 mg, at least 0.6 mg, at least 0.7 mg, at least 0.8 mg, at least 0.9, at least 1.0 mg, at least 2.0 mg, at least 3.0 mg, at least 4.0 mg, at least 5.0 mg, at least 10 mg, at least 15 mg, or at least 20 mg stabilizer(s) per unit dose. In some embodiments, the dosage comprises at most 0.45 mg stabilizer per unit dose. In some embodiments, the dosage comprises at most 0.1 mg, at most 0.2 mg, at most 0.3 mg, at most 0.4 mg, at most 0.5 mg, at most 0.6 mg, at most 0.7 mg, at most 0.8 mg, at most 0.9, at most 1.0 mg, at most 2.0 mg, at most 3.0 mg, at most 4.0 mg, at most 5.0 mg, at most 10 mg, at most 15 mg, or at most 20 mg stabilizer(s) per unit dose.

In some embodiments, such as orally formulated dosages, the dosage comprises at least 3.0 mg of an adhesion agent per unit dose. In some embodiments, the dosage comprises at least 0.1 mg, at least 0.2 mg, at least 0.3 mg, at least 0.4 mg, at least 0.5 mg, at least 0.6 mg, at least 0.7 mg, at least 0.8 mg, at least 0.9, at least 1.0 mg, at least 2.0 mg, at least 3.0 mg, at least 4.0 mg, at least 5.0 mg, at least 10 mg, at least 15 mg, or at least 20 mg stabilizer(s) per unit dose. In some embodiments, the dosage comprises at most 0.45 mg of an adhesion agent per unit dose. In some embodiments, the dosage comprises at most 0.1 mg, at most 0.2 mg, at most 0.3 mg, at most 0.4 mg, at most 0.5 mg, at most 0.6 mg, at most 0.7 mg, at most 0.8 mg, at most 0.9, at most 1.0 mg, at most 2.0 mg, at most 3.0 mg, at most 4.0 mg, at most 5.0 mg, at most 10 mg, at most 15 mg, or at most 20 mg of an adhesion agent per unit dose.

In some embodiments, the unit dosage comprises at least 1.0% lactic acid-producing compound by dry weight. In some embodiments, the unit dosage comprises at least 25.0% lactic acid-producing compound by dry weight. In some embodiments, the unit dosage comprises at least 50% lactic acid-producing compound by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more lactic acid-producing compound by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, or at most 95% lactic acid-producing compound by dry weight.

In some embodiments, the unit dosage comprises at least 10.0% excipient(s) by dry weight. In some embodiments, the unit dosage comprises at least 47.2% excipient(s) by dry weight. In some embodiments, the unit dosage comprises at least 70% excipient(s) by dry weight. In some embodiments, the unit dosage comprises at least 72% excipient(s) by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more excipient(s) by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% excipient(s) by dry weight.

In some embodiments, the unit dosage comprises at least 5.0% of a first excipient and at least 5.0% of a second excipient. In some embodiments, the unit dosage comprises at least 23.75% of a first excipient and at least 23.75% of a second excipient. In some embodiments, the unit dosage comprises at least 36% of a first excipient and at least 36% of a second excipient. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of a first excipient by dry weight, and at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of a second excipient by dry weight.

In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of a first excipient by dry weight, and at least 5.0% of a second excipient by dry weight. In some embodiments, the unit dosage comprises at least 5.0% of a first excipient by dry weight, and at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of a second excipient by dry weight.

In some embodiments, the unit dosage comprises at least 0.1% stabilizer(s) by dry weight. In some embodiments, the unit dosage comprises at least 1.5% stabilizer(s) by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more stabilizer(s) by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% stabilizer(s) by dry weight.

In some embodiments, the unit dosage comprises at least 1.5% of an adhesion agent by dry weight. In some embodiments, the unit dosage comprises at least 0.5%, at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more of an adhesion agent by dry weight. In some embodiments, the unit dosage comprises at most 0.5%, at most 1%, at most 2%, at most 3%, at most 4%, at most 5%, at most 6%, at most 7%, at most 8%, at most 9%, at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% of an adhesion agent by dry weight.

In some embodiments, the pharmaceutical composition is a spray-dried pharmaceutical composition. In some embodiments, the pharmaceutical composition comprises a spray-dried composition. In some embodiments, the pharmaceutical composition consults essentially of a spray-dried composition.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of a chronic bronchopulmonary disease. A subject can be male or female.

A subject can be one who has been previously diagnosed with or identified as suffering from or having a condition in need of treatment (e.g. a chronic bronchopulmonary disease) or one or more complications related to such a condition, and optionally, have already undergone treatment for a chronic bronchopulmonary disease or the one or more complications related to a chronic bronchopulmonary disease. Alternatively, a subject can also be one who has not been previously diagnosed as having a chronic bronchopulmonary disease or one or more complications related to a chronic bronchopulmonary disease. For example, a subject can be one who exhibits one or more risk factors for a chronic bronchopulmonary disease or one or more complications related to a chronic bronchopulmonary disease or a subject who does not exhibit risk factors.

A “subject in need” of treatment for a particular condition can be a subject having that condition, diagnosed as having that condition, or at risk of developing that condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a chronic bronchopulmonary disease. The term “treating” includes reducing or alleviating at least one adverse effect or symptom of a condition, disease or disorder associated with a chronic bronchopulmonary disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable excipient, stabilizer, and/or additive e.g. an excipient, stabilizer, and/or additive commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable excipient, stabilizer, and/or additive can be an excipient, stabilizer, and/or additive other than water. In some embodiments of any of the aspects, a pharmaceutically acceptable excipient, stabilizer, and/or additive can be an artificial or engineered excipient, stabilizer, and/or additive, e.g., an excipient, stabilizer, and/or additive that the active ingredient would not be found to occur in or within nature.

“Pharmaceutically acceptable” excipients, stabilizers, and/or additives are those which can reasonably be administered to a subject to provide an effective dose of the active ingredient employed (e.g., a lactic acid-producing compound). In some embodiments, these are excipients which the Federal Drug Administration (FDA) have to date designated as ‘Generally Regarded as Safe’ (GRAS).

“Powder” means a composition that consists of finely dispersed solid particles that are relatively free flowing and capable of being readily dispersed in an inhalation device and subsequently inhaled by a patient so that the particles are suitable for intranasal or pulmonary administration via the upper respiratory tract.

The “glass transition temperature” is represented by the symbol T_{g} and is the temperature at which a composition changes from a glassy or vitreous state to a syrup or rubbery state. Generally, T_{g} is determined using differential scanning calorimetry (DSC) and is standardly taken as the temperature at which onset of the change of heat capacity (Cp) of the composition occurs upon scanning through the transition. The definition of T_{g} is always arbitrary and there is no present international convention. The T_{g} can be defined as the onset, midpoint or endpoint of the transition. See the article entitled “Formation of Glasses from Liquids and Biopolymers” by C. A. Angell: Science, 267, 1924-1935 (Mar. 31, 1995) and the article entitled “Differential Scanning Calorimetry Analysis of Glass Transitions” by Jan P. Wolanczyk: Cryo-Letters, 10, 73-76 (1989). For detailed mathematical treatment see “Nature of the Glass Transition and the Glassy State” by Gibbs and DiMarzio: Journal of Chemical Physics, 28, NO. 3, 373-383 (March, 1958). These articles are incorporated herein by reference.

A “stable” formulation or composition is one in which the active material therein (e.g., the lactic acid-producing compound) essentially retains its physical stability and/or chemical stability and/or biological activity upon storage. Various analytical techniques for measuring stability are available in the art and are reviewed, e.g., in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993). Stability can be measured at a selected temperature for a selected time period. Trend analysis can be used to estimate an expected shelf life before a material has actually been in storage for that time period. In some embodiments, the composition is stable at room temperature (−25° C.) for at least 3 months, and or stable at about 2-8° C. for at least 1 year. In some embodiments, the composition is stable following freezing (to, e.g., −70° C.) and thawing of the composition.

“Unit dosage” or “unit dose” refers to a receptacle containing a therapeutically effective amount of a pharmaceutical composition as described herein, designed or formulated for administration in a single dose.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an act of inhalation, an act of ingestion, and/or manipulation of a delivery device or machine. Such activity can be performed, e.g., by a medical professional and/or the subject being treated.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A pharmaceutical composition comprising:
   a) a lactic acid-producing compound selected from:
      i) a polymeric compound that can produce lactic acid;
      ii) a non-polymeric compound that can produce lactic acid; or
      iii) lactic acid; and
   b) a pharmaceutically acceptable excipient, stabilizer, or additive; wherein the composition is formulated for administration to the lungs.
2. The pharmaceutical composition of paragraph 1, wherein the composition is formulated for administration by inhalation.
3. The pharmaceutical composition of paragraph 1 or 2, wherein the lactic acid-producing compound produces lactic acid upon delivery to a target tissue.
4. The pharmaceutical composition of paragraph 3, wherein the target tissue is a target bronchopulmonary tissue.
5. The pharmaceutical composition of paragraph 4, wherein the target bronchopulmonary tissue is the lungs, the trachea, the bronchi, the bronchioles, and/or the alveoli.
6. The pharmaceutical composition of paragraph 4, wherein the target tissue is a distal tissue site from the lungs delivered via the cardiovascular system or lymphatic system.
7. The pharmaceutical composition of any one of paragraphs 1-6, wherein the lactic acid-producing compound comprises a D enantiomer of lactic acid, an L enantiomer of lactic acid, or a racemic mixture of D and L enantiomers of lactic acid.
8. The pharmaceutical composition of any one of paragraphs 1-7, wherein the non-polymeric lactic acid-producing compound can be metabolized in the target tissue to produce lactic acid.
9. The pharmaceutical composition of any one of paragraphs 1-8, wherein the non-polymeric lactic acid-producing compound is an inorganic salt of lactic acid, an ester of lactic acid, or lactide.
10. The pharmaceutical composition of paragraph 9, wherein the inorganic salt of lactic acid is sodium lactate, potassium lactate, calcium lactate, or magnesium lactate.
11. The pharmaceutical composition of paragraph 9, wherein the ester of lactic acid is ethyl lactate, propyl lactate, butyl lactate, pentyl lactate, hexyl lactate, heptyl lactate, octyl lactate, nonyl lactate, decyl lactate, undecyl lactate, or dodecyl lactate.
12. The pharmaceutical composition of any one of paragraphs 1-11, wherein the polymeric lactic acid-producing compound can be hydrolyzed in the target tissue to produce lactic acid.
13. The pharmaceutical composition of any one of paragraphs 1-12, wherein the polymeric lactic acid-producing compound is a polylactic acid (PLA).
14. The pharmaceutical composition of any one of paragraphs 1-13, wherein the polylactic acid is poly(L-lactide) (PLLA), poly(D,L-lactide) (PDLLA), or poly(D-lactide) (PDLA).
15. The pharmaceutical composition of any one of paragraphs 1-14, wherein the polylactic acid is poly(D,L-lactide) (PDLLA).
16. The pharmaceutical composition of any one of paragraphs 1-15, wherein the polymeric lactic acid-producing compound is poly(lactic-co-glycolic acid) (PLGA).
17. The pharmaceutical composition of any one of paragraphs 1-16, wherein the polymeric lactic acid-producing compound is poly(lactic acid-co-caprolactone).
18. The pharmaceutical composition of any one of paragraphs 1-17, wherein the composition comprises at least 1.0% lactic acid-producing compound by weight.
19. The pharmaceutical composition of any one of paragraphs 1-18, wherein the composition comprises at least one excipient or at least one stabilizer.
20. The pharmaceutical composition of any one of paragraphs 1-19, further comprising at least one excipient.
21. The pharmaceutical composition of any one of paragraphs 1-20, further comprising at least two excipients.
22. The pharmaceutical composition of paragraph 20 or 21, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcamitine; propionylcamitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.
23. The pharmaceutical composition of any one of paragraphs 20-22, wherein the excipient is leucine and/or trehalose.
24. The pharmaceutical composition of any one of paragraphs 1-23, wherein the composition comprises at least 5.0% excipient by weight.
25. The pharmaceutical composition of any one of paragraphs 1-24, wherein the composition comprises at least 5.0% of a first excipient by weight, and at least 5.0% of a second excipient by weight.
26. The pharmaceutical composition of any one of paragraphs 1-25, further comprising at least one stabilizer.
27. The pharmaceutical composition of paragraph 26, wherein the stabilizer comprises a surfactant.
28. The pharmaceutical composition of paragraph 26 or 27, wherein the stabilizer is selected from the group consisting of: mannitol, carboxymethyl cellulose (CMC), polyvinyl alcohol (PVA), polysorbate, and poloxamer.
29. The pharmaceutical composition of paragraph 26 or 27, wherein the stabilizer is a polysorbate; poloxamer; or polyvinyl alcohol.
30. The pharmaceutical composition of any one of paragraphs 26-29, wherein the stabilizer is Polysorbate 20, Polysorbate 40, Polysorbate 60, or Polysorbate 80.
31. The pharmaceutical composition of any one of paragraphs 26-30, wherein the stabilizer is Polysorbate 80.
32. The pharmaceutical composition of any one of paragraphs 26-31, wherein the stabilizer is Poloxamer 184, Poloxamer 185, Poloxamer 188, Poloxamer 234, Poloxamer 235, Poloxamer 238, Poloxamer 333, Poloxamer 334, Poloxamer 335, Poloxamer 338, Poloxamer 403, or Poloxamer 407.
33. The pharmaceutical composition of any one of paragraphs 26-32, wherein the stabilizer is poloxamer 188.
34. The pharmaceutical composition of any one of paragraphs 1-33, wherein the composition comprises at least 0.10% stabilizer by weight.
35. The pharmaceutical composition of any one of paragraphs 1-34, wherein the composition comprises at least one excipient and at least one stabilizer.
36. The pharmaceutical composition of any one of paragraphs 1-35, further comprising at least one of the following:
   a) a pore-forming agent;
   b) an adhesion agent;
   c) a pH-modulating agent; and/or
   d) an ester-hydrolysis-inducing agent.
37. The pharmaceutical composition of paragraph 36, wherein the pore-forming agent is selected from the group consisting of: NaCl, sucrose, polyethylene glycol (PEG), and polyvinylpyrrolidone (PVP).
38. The pharmaceutical composition of paragraph 36, wherein the adhesion agent is selected from the group consisting of: sugars, adhesive polymers, and amine-containing compounds.
39. The pharmaceutical composition of paragraph 36, wherein the pH-modulating agent is a buffer, an acid or a base.
40. The pharmaceutical composition of paragraph 36, wherein the ester-hydrolysis-inducing agent comprises an amine.
41. The pharmaceutical composition of any one of paragraphs 1-40, further comprising at least one acid-generating molecule that can generate acid, wherein the at least one acid-generating molecule is not lactic acid or does not comprise lactic acid.
42. The pharmaceutical composition of paragraph 41, wherein the acid-generating molecule is selected from the group consisting of: acetic acid, a hydroxy acid, a multiple functional acid, and an aromatic acid, and esters, salts, and polymers thereof.
43. The pharmaceutical composition of paragraph 41, wherein the acid-generating molecule is selected from the group consisting of: acetic acid, glycolic acid, citric acid, and salicylic acid, and esters, salts, and polymers thereof.
44. The pharmaceutical composition of any one of paragraphs 41-43, wherein the acid-generating molecule is acetylsalicylic acid.
45. The pharmaceutical composition of any one of paragraphs 1-45, wherein the composition comprises at least one additional therapeutic for a chronic bronchopulmonary disorder.
46. The pharmaceutical composition of paragraph 45, wherein the at least one additional therapeutic is microencapsulated.
47. The pharmaceutical composition of paragraph 45, wherein the at least one additional therapeutic is covalently linked with a degradable linker to the lactic acid-producing compound.
48. The pharmaceutical composition of any one of paragraphs 1-47, co-administered with at least one additional therapeutic for a chronic or infectious bronchopulmonary disorder.
49. The pharmaceutical composition of paragraph 48, wherein the at least one additional therapeutic is an anti-inflammatory, an antimicrobial, an antiviral, an antifungal, a vasodilator, or a bronchodilator.
50. The pharmaceutical composition of paragraph 49, wherein the anti-inflammatory is selected from the group consisting of: non-steroidal anti-inflammatory drugs (NSAIDs); corticosteroids; glucocorticoids; methotrexate; sulfasalazine; leflunomide; anti-tumor necrosis factor (TNF) medications; cyclophosphamide; pro-resolving lipid mediators; mycophenolate; opiates; and barbiturates.
51. The pharmaceutical composition of paragraph 49, wherein the antimicrobial is selected from the group consisting of: aminoglycosides; ansamycins; beta-lactams; bis-biguanides; carbacephems; carbapenems; cationic polypeptides; cephalosporins; fluoroquinolones; glycopeptides; iron-sequestering glycoproteins; linosamides; lipopeptides; macrolides; monobactams; nitrofurans; oxazolidinones; penicillins; polypeptides; quaternary ammonium compounds; quinolones; silver compounds; sulfonamides; and tetracyclines.
52. The pharmaceutical composition of paragraph 49, wherein the vasodilator is selected from the group consisting of: an angiotensin converting enzyme (ACE) inhibitor; an angiotensin receptor blocker (ARB); a calcium channel blocker (CCB); and a nitric-oxide-producing compound.
53. The pharmaceutical composition of paragraph 49, wherein the bronchodilator is selected from the group consisting of: albuterol, levalbuterol, epinephrine, salmeterol, formoterol, ipratropium bromide, tiotropium bromide, theophylline, and aminophylline.
54. The pharmaceutical composition of any one of paragraphs 1-53, wherein the composition is formulated as a bolus dose.
55. The pharmaceutical composition of any one of paragraphs 1-54, comprising:
   a) the polymeric lactic acid-producing compound and/or the non-polymeric lactic acid-producing compound; and
   b) a bolus dose of lactic acid.
56. The pharmaceutical composition of any one of paragraphs 1-55, wherein the composition is formulated as microspheres.
57. The pharmaceutical composition of paragraph 56, wherein the microspheres have a diameter of at least 1 μm to at most 1 mm.
58. The pharmaceutical composition of any one of paragraphs 1-57, wherein the composition comprises a plurality of dried particles.
59. The pharmaceutical composition of paragraph 58, wherein the dried particles have a Dv50 of at least 0.5 μm.
60. The pharmaceutical composition of paragraph 58 or 59, wherein the dried particles have a median mass aerodynamic diameter (MMAD) of at least 1.5 μm to at most 7.5 μm.
61. The pharmaceutical composition of any one of paragraphs 58-60, wherein the dried particles have a median mass aerodynamic diameter (MMAD) of at most 5.0 μm.
62. The pharmaceutical composition of any one of paragraphs 58-61, wherein the dried particles have a dispersibility of less than 2.0.
63. The pharmaceutical composition of any one of paragraphs 58-62, wherein the dried particles have a dispersibility of at least 0.5 to 1.0.
64. The pharmaceutical composition of any one of paragraphs 58-63, wherein the dried particles have a dispersibility of at least 0.9.
65. The pharmaceutical composition of any one of paragraphs 58-64, wherein the dried particles have a delivered dose of at least 25.0% to at most 125% of the composition by mass to a target tissue.
66. The pharmaceutical composition of any one of paragraphs 58-65, wherein the dried particles have a delivered dose of at least 30% of the lactic acid-producing compound by mass to a target tissue.
67. The pharmaceutical composition of any one of paragraphs 58-66, wherein the dried particles have a delivered dose of at least 7.8 mg per unit dose of the lactic acid-producing compound by mass to a target tissue.
68. The pharmaceutical composition of any one of paragraphs 58-67, wherein the dried particles have a delivered dose of at most 50 mg per unit dose of the lactic acid-producing compound by mass to a target tissue.
69. The pharmaceutical composition of any one of paragraphs 58-68, wherein the dried particles have a bulk density of at least 0.1 g/cm³ to 0.8 g/cm³.
70. The pharmaceutical composition of any one of paragraphs 58-69, wherein the dried particles have a bulk density of at least 0.5 g/cm³.
71. The pharmaceutical composition of any one of paragraphs 58-70, wherein the dried particles have a tapped density of at least 0.2 g/cm³ to 1.0 g/cm³.
72. The pharmaceutical composition of any one of paragraphs 58-71, wherein the dried particles have a tapped density of at least 0.6 g/cm³.
73. The pharmaceutical composition of any one of paragraphs 58-72, wherein the dried particles have a moisture content of at least 1.0% to 7.0% water by weight.
74. The pharmaceutical composition of any one of paragraphs 58-73, wherein the dried particles have a moisture content of at least 2.3% water by weight.
75. The pharmaceutical composition of any one of paragraphs 1-74, wherein the composition is formulated for delivery to the trachea, the bronchi, the bronchioles, and/or the alveoli.
76. The pharmaceutical composition of any one of paragraphs 1-75, wherein the composition is formulated for delivery to the lungs.
77. The pharmaceutical composition of any one of paragraphs 1-76, wherein the composition is formulated as a capsule or a tablet.
78. The pharmaceutical composition of any one of paragraphs 1-77, wherein the composition comprises at least 7.8 mg of the lactic acid-producing compound per unit dose that is deliverable to a target tissue.
79. The pharmaceutical composition of any one of paragraphs 1-78, wherein the composition comprises at least 15 mg of the lactic acid-producing compound per unit dose.
80. The pharmaceutical composition of paragraph 79, wherein the composition is formulated for delivery by an inhaler.
81. The pharmaceutical composition of paragraph 80, wherein the composition is formulated for delivery by a dry powder inhaler (DPI).
82. The pharmaceutical composition of paragraph 80, wherein the composition is formulated for delivery by a metered dose inhaler (MDI).
83. The pharmaceutical composition of paragraph 80, wherein the composition is formulated for delivery by a soft mist inhaler (SMI).
84. The pharmaceutical composition of any one of paragraphs 1-83, in combination with an inhaler.
85. An inhalation device for bronchopulmonary delivery comprising:
   a) an inhaler; and
   b) a container containing the pharmaceutical composition of any one of paragraphs 1-83.
86. The device of paragraph 85, wherein the inhaler is a dry powder inhaler (DPI).
87. The device of paragraph 85, wherein the inhaler is a metered dose inhaler (MDI).
88. The device of paragraph 85, wherein the inhaler is a soft mist inhaler (SMI).
89. The device of any one of paragraphs 85-88, wherein the inhaler comprises:
   a) a mouthpiece comprising an opening; and
   b) means for aerosolizing or dispersing the pharmaceutical composition in the container.
90. A method of preparing a spray-dried pharmaceutical composition comprising a lactic acid-producing compound, comprising:
   a) preparing a liquid feedstock comprising the lactic acid-producing compound;
   b) introducing droplets of the liquid feedstock through an atomization nozzle into a drying chamber;
   c) exposing the liquid feedstock droplets to heated, pressurized gas in the drying chamber to create dried particles; and
   d) isolating dried particles of a predetermined range of diameters in a cyclone chamber, wherein the isolated dried particles comprise the lactic acid-producing compound.
91. A method of preparing a spray-dried pharmaceutical composition comprising a lactic acid-producing compound, comprising:
   a) obtaining a liquid feedstock comprising the lactic acid-producing compound;
   b) introducing droplets of the liquid feedstock through an atomization nozzle into a drying chamber;
   c) exposing the liquid feedstock droplets to heated, pressurized gas in the drying chamber to create dried particles; and
   d) isolating dried particles of a predetermined range of diameters in a cyclone chamber, wherein the isolated dried particles comprise the lactic acid-producing compound.
92. The method of paragraph 90 or 91, wherein the step of preparing the liquid feedstock comprises dissolving a solid feedstock into an aqueous solution.
93. The method of any one of paragraphs 90-92, wherein the step of preparing the liquid feedstock comprises dissolving a solid feedstock into an organic solution.
94. The method of any one of paragraphs 90-93, wherein the step of preparing the liquid feedstock comprises:
   a) dissolving a lactic acid-producing compound into an organic solution;
   b) dissolving a solid feedstock into an aqueous solution; and
   c) combining the resultant solutions from (a) and (b) to produce a liquid feedstock.
95. The method of any one of paragraphs 90-94, wherein the solid feedstock comprises:
   a) at least 50% lactic acid-producing compound by weight;
   b) at least 10% excipient by weight; and/or
   c) at least 1% stabilizer by weight.
96. The method of any one of paragraphs 90-95, wherein the solid feedstock comprises:
   a) at least 50% lactic acid-producing compound by weight;
   b) at least 5% of a first excipient by weight;
   c) at least 5% of a second excipient by weight; and/or
   d) at least 1% stabilizer by weight.
97. The method of any one of paragraphs 90-96, wherein the solid feedstock comprises at least 20% to at most 80% lactic acid-producing compound by weight.
98. The method of any one of paragraphs 90-97, wherein the solid feedstock comprises at least 1% to at most 15% excipient by weight.
99. The method of any one of paragraphs 90-98, wherein the solid feedstock comprises at least 2.5%-7.5% of a first excipient by weight, and at least 2.5%-7.5% of a second excipient by weight.
100. The method of any one of paragraphs 90-99, wherein the solid feedstock comprises at least 10% to at most 50% stabilizer by weight.
101. The method of any one of paragraphs 90-100, wherein the liquid feedstock comprises at least 0.1 g/L solid feedstock dissolved in an aqueous solution.
102. The method of any one of paragraphs 90-101, wherein the liquid feedstock comprises at least 5 g/L solid feedstock dissolved in an aqueous solution.
103. The method of any one of paragraphs 90-102, wherein the liquid feedstock comprises at least 0.01% to at most 10% solid feedstock dissolved in an aqueous solution.
104. The method of any one of paragraphs 90-103, wherein the liquid feedstock comprises at least 0.5% solid feedstock dissolved in an aqueous solution.
105. The method of any one of paragraphs 90-104, wherein 0.5 L of the liquid feedstock comprises:
   a) at least 2.00 g lactic acid-producing compound;
   b) at least 0.80 g excipient;
   c) at least 1.2 g stabilizer;
   d) at least 31.36 g organic solution; and/or
   e) at least 464.64 g aqueous solution.
106. The method of any one of paragraphs 90-105, wherein 0.5 L of the liquid feedstock comprises:
   a) at least 2.00 g lactic acid-producing compound;
   b) at least 0.40 g of a first excipient;
   c) at least 0.40 g of a second excipient;
   d) at least 1.2 g stabilizer;
   e) at least 31.36 g organic solution; and/or
   f) at least 464.64 g aqueous solution.
107. The method of any one of paragraphs 90-106, wherein 0.5 L of the liquid feedstock comprises at least 0.1 g to at most 10 g lactic acid-producing compound.
108. The method of any one of paragraphs 90-107, wherein 0.5 L of the liquid feedstock comprises at least 0.1 g to at most 10 g excipient.
109. The method of any one of paragraphs 90-108, wherein 0.5 L of the liquid feedstock comprises at least 0.05 g to at most 5 g of a first excipient, and at least 0.05 g to at most 5 g of a second excipient.
110. The method of any one of paragraphs 90-109, wherein 0.5 L of the liquid feedstock comprises at least 0.1 g to at most 10 g stabilizer.
111. The method of any one of paragraphs 90-110, wherein 0.5 L of the liquid feedstock comprises at least 10 g to at most 50 g organic solution.
112. The method of any one of paragraphs 90-111, wherein 0.5 L of the liquid feedstock comprises at least 420 g to at most 490 g aqueous solution.
113. The method of any one of paragraphs 90-112, wherein the liquid feedstock comprises:
   a) at least 0.40% lactic acid-producing compound;
   b) at least 0.16% excipient;
   c) at least 0.24% stabilizer;
   d) at least 6.27% organic solution; and/or
   e) at least 92.93% aqueous solution.
114. The method of any one of paragraphs 90-113, wherein the liquid feedstock comprises:
   a) at least 0.40% lactic acid-producing compound by weight;
   b) at least 0.08% of a first excipient by weight;
   c) at least 0.08% of a second excipient by weight;
   d) at least 0.24% stabilizer;
   e) at least 6.27% organic solution; and/or
   f) at least 92.93% aqueous solution by weight.
115. The method of any one of paragraphs 90-114, wherein the liquid feedstock comprises at least 0.01% to at most 1.0% lactic acid-producing compound by weight.
116. The method of any one of paragraphs 90-115, wherein the liquid feedstock comprises at least 0.01% to at most 10% excipient by weight.
117. The method of any one of paragraphs 90-116, wherein the liquid feedstock comprises at least 0.005% to at most 5% of a first excipient by weight, and at least 0.005% to at most 5% of a second excipient by weight.
118. The method of any one of paragraphs 90-117, wherein the liquid feedstock comprises at least 0.01% to at most 1.0% stabilizer by weight.
119. The method of any one of paragraphs 90-118, wherein the liquid feedstock comprises at least 1% to at most 5% organic solution by weight.
120. The method of any one of paragraphs 90-119, wherein the liquid feedstock comprises at least 90% to at most 99.9% aqueous solution by weight.
121. The method of any one of paragraphs 90-120, wherein the lactic acid-producing compound is selected from:
   a) a polymeric compound that can produce lactic acid;
   b) a non-polymeric compound that can produce lactic acid; or
   c) lactic acid.
122. The method of any one of paragraphs 90-121, wherein the non-polymeric lactic acid-producing compound is an inorganic salt of lactic acid, an ester of lactic acid, or lactide.
123. The method of any one of paragraphs 90-122, wherein the polymeric lactic acid-producing compound is a polylactic acid (PLA).
124. The method of any one of paragraphs 90-123, wherein the polylactic acid is poly(D,L-lactide) (PDLLA).
125. The method of any one of paragraphs 90-124, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcamitine; propionylcamitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.
126. The method of any one of paragraphs 90-125, wherein the excipient is leucine and/or trehalose.
127. The method of any one of paragraphs 90-126, wherein the stabilizer is a polysorbate; poloxamer; or polyvinyl alcohol.
128. The method of any one of paragraphs 90-127, wherein the stabilizer is Poloxamer 188.
129. The method of any one of paragraphs 90-128, wherein the organic solution is acetone.
130. The method of any one of paragraphs 90-129, wherein the aqueous solution is water.
131. The method of any one of paragraphs 90-130, wherein the liquid feedstock further comprises at least one additional therapeutic.
132. The method of paragraph 131, wherein the at least one additional therapeutic is selected from the group consisting of: an anti-inflammatory, an antimicrobial, an antiviral, an antifungal, a vasodilator, and a bronchodilator.
133. The method of any one of paragraphs 90-132, wherein the atomization nozzle into the drying chamber has a diameter of at least 1.2 mm.
134. The method of any one of paragraphs 90-133, wherein the droplets of liquid feedstock produced by the atomization nozzle into the drying chamber have a diameter of at least 1.2 um.
135. The method of any one of paragraphs 90-134, wherein the droplets of liquid feedstock have a flow rate through the drying chamber of at least 5 g/min.
136. The method of any one of paragraphs 90-135, wherein the droplets of liquid feedstock have a flow rate through the drying chamber of at least 15 g/min.
137. The method of any one of paragraphs 90-136, wherein the droplets of liquid feedstock have a flow rate through the drying chamber of at most 1000 g/min.
138. The method of any one of paragraphs 90-137, wherein the heated, pressurized gas is heated before being inlet into the drying chamber.
139. The method of any one of paragraphs 90-138, wherein the heated, pressurized gas is inlet into the drying chamber at a temperature of at least 100° C.
140. The method of any one of paragraphs 90-139, wherein the heated, pressurized gas is inlet into the drying chamber at a temperature of at least 135° C.
141. The method of any one of paragraphs 90-140, wherein the heated, pressurized gas is inlet into the drying chamber at a temperature of at most 195° C.
142. The method of any one of paragraphs 90-141, wherein the heated, pressurized gas is outlet from the drying chamber at a temperature of at least 40° C.
143. The method of any one of paragraphs 90-142, wherein the heated, pressurized gas is outlet from the drying chamber at a temperature of at least 60° C.
144. The method of any one of paragraphs 90-143, wherein the heated, pressurized gas is outlet from the drying chamber at a temperature of at most 85° C.
145. The method of any one of paragraphs 90-144, wherein the heated, pressurized gas is pressurized before being inlet into the drying chamber.
146. The method of any one of paragraphs 90-145, wherein the heated, pressurized gas in the drying chamber has an atomization gas pressure of at least 10 pounds per square inch gauge (psig).
147. The method of any one of paragraphs 90-146, wherein the heated, pressurized gas in the drying chamber has an atomization gas pressure of at least 20 pounds per square inch gauge (psig).
148. The method of any one of paragraphs 90-147, wherein the heated, pressurized gas in the drying chamber has an atomization gas pressure of at most 150 pounds per square inch gauge (psig).
149. The method of any one of paragraphs 90-148, wherein the heated, pressurized gas has a flow rate through the drying chamber of at least 5 kg/hr.
150. The method of any one of paragraphs 90-149, wherein the heated, pressurized gas has a flow rate through the drying chamber of at least 18 kg/hr.
151. The method of any one of paragraphs 90-150, wherein the heated, pressurized gas has a flow rate through the drying chamber of at most 150 kg/hr.
152. The method of any one of paragraphs 90-151, wherein the heated, pressurized gas is outlet through the cyclone chamber.
153. The method of any one of paragraphs 90-152, wherein the step of exposing the liquid feedstock droplets to heated, pressurized gas in the drying chamber takes at most 8 hours.
154. The method of any one of paragraphs 90-153, wherein the dried particles isolated in the cyclone chamber have a median mass aerodynamic diameter (MMAD) of at least 1.5 μm to at most 7.5 μm.
155. The method of any one of paragraphs 90-154, wherein the dried particles isolated in the cyclone chamber have a median mass aerodynamic diameter (MMAD) of at least 4.0 μm.
156. The method of any one of paragraphs 90-155, wherein the dried particles isolated in the cyclone chamber have a median mass aerodynamic diameter (MMAD) of at most 5.0 μm.
157. The method of any one of paragraphs 90-156, wherein the step of isolating dried particles of a predetermined range of diameters in the cyclone chamber occurs continuously.
158. A method of delivering a spray-dried pharmaceutical composition comprising a lactic acid-producing compound to a subject, comprising:
   a) obtaining an inhalation device for bronchopulmonary delivery comprising:
      i) an inhaler; and
      ii) a container containing a spray-dried pharmaceutical composition comprising a lactic acid-producing compound;
   b) activating the inhaler to cause aerosolization or dispersal of the spray-dried pharmaceutical composition; and
   c) inhaling the aerosolized or dispersed spray-dried pharmaceutical composition.
159. A method of delivering a spray-dried pharmaceutical composition comprising a lactic acid-producing compound to a subject, comprising:
   a) obtaining an inhalation device for bronchopulmonary delivery comprising:
      i) an inhaler; and
      ii) a container containing the pharmaceutical composition of any one of paragraphs 1-84, wherein the pharmaceutical composition is spray-dried;
   b) activating the inhaler to cause aerosolization or dispersal of the spray-dried pharmaceutical composition; and
   c) inhaling the aerosolized or dispersed spray-dried pharmaceutical composition.
160. A method of delivering a spray-dried pharmaceutical composition comprising a lactic acid-producing compound to a subject, comprising:
   a) obtaining the inhalation device of any one of paragraphs 85-89;
   b) activating the inhaler to cause aerosolization or dispersal of the spray-dried pharmaceutical composition; and
   c) inhaling the aerosolized or dispersed spray-dried pharmaceutical composition.
161. The method of any one of paragraphs 158-160, wherein the inhaler is a dry powder inhaler (DPI).
162. The method of any one of paragraphs 158-161, wherein the inhaler is a metered dose inhaler (MDI).
163. The method of any one of paragraphs 158-162, wherein the inhaler is a soft mist inhaler (SMI).
164. The method of any one of paragraphs 158-163, wherein the inhaler comprises:
   a) a mouthpiece comprising an opening; and
   b) means for aerosolizing or dispersing the spray-dried pharmaceutical composition in the container.
165. The method of any one of paragraphs 158-164, wherein the inhaler has an inspiration flow rate of at least 15 L/min to at most 60 L/min.
166. The method of any one of paragraphs 158-165, wherein at least 25% to at most 125% of the spray-dried pharmaceutical composition by mass is delivered to a target bronchopulmonary tissue.
167. The method of any one of paragraphs 158-166, wherein at least 30% of the spray-dried pharmaceutical composition by mass is delivered to a target bronchopulmonary tissue.
168. The method of any one of paragraphs 158-167, wherein the target bronchopulmonary tissue is the lungs, the trachea, the bronchi, the bronchioles, and/or the alveoli.
169. The method of any one of paragraphs 158-168, wherein the spray-dried pharmaceutical composition is delivered from the bronchopulmonary tissue to a distal tissue site via the cardiovascular system or lymphatic system.
170. A method of treating a subject in need thereof comprising administering through inhalation an effective dose of a pharmaceutical composition comprising a lactic acid-producing compound.
171. A method of treating a subject in need thereof comprising administering through inhalation an effective dose of the pharmaceutical composition of any one of paragraphs 1-84.
172. The method of paragraph 170 or 171, wherein the lactic acid-producing compound reduces neutrophilic inflammation in a target tissue.
173. The method of paragraph 172, wherein the target tissue is a target bronchopulmonary tissue.
174. The method of paragraph 173, wherein the target bronchopulmonary tissue is the lungs, the trachea, the bronchi, the bronchioles, and/or the alveoli.
175. The method of paragraphs 172, wherein the target tissue is a distal tissue site from the lungs delivered via the cardiovascular system or lymphatic system.
176. The method of any one of paragraphs 170-175, wherein the subject has been diagnosed with or is at risk of developing a chronic or infectious bronchopulmonary disease.
177. The method of paragraph 176, wherein the chronic bronchopulmonary disease is selected from the group consisting of: asthma, bronchopulmonary dysplasia (BPD), chronic obstructive pulmonary disease (COPD), bronchiectasis, non-cystic fibrosis (CF) bronchiectasis, cystic fibrosis (CF), acute respiratory distress syndrome (ARDS), idiopathic pulmonary fibrosis (IPF), interstitial lung disease (LD), pleural effusion (PE), pulmonary hypertension (PAH), silicosis, and lung cancer.
178. The method of any one of paragraphs 170-177, wherein the lung cancer is small cell lung cancer (SCLC) or non-small cell lung cancer (NSCLC).
179. The method of paragraph 178, wherein the infectious bronchopulmonary disease is caused by or associated with an infectious agent selected from: adenovirus; coronavirus; influenza virus; parainfluenza virus; parvovirus; respiratory syncytial virus; rhinovirus; enterovirus; measles virus; rubella virus; varicella virus; Corynebacterium diphtheriae; Haemophilus influenzae; Legionella pneumophila; Bordetella pertussis; Mycobacterium tuberculosis; Streptococcus species; Pseudomonas species; Escherichia coli; Aspergillus species; Cryptococcus species; and Pneumocystis species.
180. The method of any one of paragraphs 170-179, wherein the pharmaceutical composition is a spray-dried pharmaceutical composition.
181. The method of any one of paragraphs 170-180, wherein the pharmaceutical composition is administered using the standard of care for the chronic or infectious bronchopulmonary disease.
182. The method of any one of paragraphs 170-181, wherein the pharmaceutical composition is administered using an inhaler.
183. The method of paragraph 182, wherein the inhaler is a dry powder inhaler (DPI).
184. The method of paragraph 182, wherein the inhaler is a metered dose inhaler (MDI).
185. The method of paragraph 182, wherein the inhaler is a soft mist inhaler (SMI).
186. The method of any one of paragraphs 170-185, wherein the effective dose of the pharmaceutical composition is at least 7.8 mg lactic acid-producing compound per unit dose.
187. The method of any one of paragraphs 170-186, wherein the pharmaceutical composition is co-administered with at least one additional therapeutic for a chronic or infectious bronchopulmonary disorder.
188. The method of paragraph 187, wherein the at least one additional therapeutic is an anti-inflammatory, an antimicrobial, an antiviral, an antifungal, a vasodilator, or a bronchodilator.
189. A unit dosage form comprising at least 1.0 mg to at most 100.0 mg of a pharmaceutical composition comprising a lactic acid-producing compound.
190. A unit dosage form comprising at least 1.0 mg to at most 100.0 mg of the pharmaceutical composition of any one of paragraphs 1-84.
191. A unit dosage form comprising at least 1.0 mg to at most 100.0 mg of the spray-dried pharmaceutical composition prepared by the methods of any one of paragraphs 90-169.
192. A unit dosage form comprising at least 15.0 mg to at most 100.0 mg of a pharmaceutical composition comprising at least 15 mg lactic acid-producing compound per unit dose.
193. The unit dosage form of any one of paragraphs 189-192, wherein the dosage is at least 1.0 mg pharmaceutical composition.
194. The unit dosage form of any one of paragraphs 189-193, wherein the dosage comprises at least 7.8 mg lactic acid-producing compound per unit dose that is deliverable to a target tissue.
195. The unit dosage form of any one of paragraphs 189-194, wherein the dosage comprises at least 15 mg lactic acid-producing compound per unit dose.
196. The unit dosage form of any one of paragraphs 189-195, wherein the pharmaceutical composition is a spray-dried pharmaceutical composition.
197. A pharmaceutical composition comprising:
   a) a lactic acid-producing compound; and
   b) a pharmaceutically acceptable excipient, stabilizer, or additive; wherein the composition is formulated for oral administration.
198. The pharmaceutical composition of paragraph 197, wherein the lactic acid-producing compound is selected from:
   a) a polymeric compound that can produce lactic acid;
   b) a non-polymeric compound that can produce lactic acid; or
   c) lactic acid.
199. The pharmaceutical composition of paragraph 197 or 198, wherein the non-polymeric lactic acid-producing compound is an inorganic salt of lactic acid, an ester of lactic acid, or lactide.
200. The pharmaceutical composition of any one of paragraphs 197-199, wherein the polymeric lactic acid-producing compound is a polylactic acid (PLA).
201. The pharmaceutical composition of paragraph 200, wherein the polylactic acid is poly(L-lactide) (PLLA), poly(D,L-lactide) (PDLLA), or poly(D-lactide) (PDLA).
202. The pharmaceutical composition of any one of paragraphs 197-201, wherein the composition comprises at least one excipient or at least one stabilizer.
203. The pharmaceutical composition of paragraph 202, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; camitine; acetylcarnitine; propionylcamitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.
204. The pharmaceutical composition of paragraph 202 or 203, wherein the excipient is leucine and/or trehalose.
205. The pharmaceutical composition of any one of paragraphs 202-204, wherein the stabilizer is a polysorbate; poloxamer; or polyvinyl alcohol.
206. The pharmaceutical composition of any one of paragraphs 202-205, wherein the stabilizer is Poloxamer 188.
207. The pharmaceutical composition of any one of paragraphs 202-206, wherein the additive is an adhesion agent.
208. The pharmaceutical composition of any one of paragraphs 197-207, wherein the composition comprises a plurality of dried particles.
209. The pharmaceutical composition of any one of paragraphs 197-208, wherein the composition comprises a plurality of spray-dried particles.
210. The pharmaceutical composition of any one of paragraphs 197-209, wherein the composition is formulated as a capsule or a tablet.
211. A method of treating a subject in need thereof comprising orally administering an effective dose of a pharmaceutical composition comprising a lactic acid-producing compound.
212. A method of treating a subject in need thereof comprising orally an effective dose of the pharmaceutical composition of any one of paragraphs 197-210.
213. The method of paragraph 211 or 212, wherein the lactic acid-producing compound reduces neutrophilic inflammation in a target tissue.
214. The method of paragraph 213, wherein the target tissue is a bronchopulmonary target tissue.
215. The method of any one of paragraphs 211-214, wherein the subject has been diagnosed with or is at risk of developing a chronic or infectious bronchopulmonary disorder.
216. The method of any one of paragraphs 211-215, wherein the pharmaceutical composition is a spray-dried pharmaceutical composition.
217. The method of any one of paragraphs 211-216, wherein the pharmaceutical composition is co-administered with at least one additional therapeutic.
218. The method of paragraph 217, wherein the at least one additional therapeutic is an anti-inflammatory, an antimicrobial, a vasodilator, or a bronchodilator.

The technology described herein is further illustrated by the following examples which in no way should be construed as being further limiting.

### EXAMPLES

### Example 1

For FIG. 1A-1B, strains AB101, AB102, AB103, and the 1:1:1 blend were measured for the amounts of L(+) and D(−) lactic acid (LA) they produced. 10⁹ CFU of each strain was cultured in MRS at 37 C in a shaker at 200 RPM for 16 hours. Cells were spun down and measured for L(+) lactic acid and for D(−) lactic acid using the Lactate Colorimetric Assay kit (MAK064, MAK058; SIGMA).

In FIG. 2, human primary epithelial (HBE) cells were seeded on a 6 well plate with 4×10⁵ cells in each well. 16 hours later, the medium was changed to fresh DULBECCO'S Modified Eagle Medium/Nutrient Mixture F-12 (DMEM/F12) without antibiotic, and E. coli was added to the cells at a final concentration of 5.0×10⁷ E. coli cells/ml. L(+) Lactic acid was added together with E. coli at a final lactic acid concentration of 5 mM, 10 mM, or 20 mM. The cells were incubated at 37° C. for 4 hours and then harvested with QIAZOL reagent. RNA was extracted according to the QIAZOL protocol. The level of Matrix Metalloproteinase-9 (MMP-9) mRNA was measured by quantitative real-time PCR (qRT-PCR).

In FIG. 3, human intestinal epithelial cells (IECs) from the Caco-2 cell line were seeded on a 6 well plate with 4×10⁵ cells in each well. 16 hours later, the medium was changed to fresh DULBECCO'S Modified Eagle Medium/Nutrient Mixture F-12 (DMEM/F12) without antibiotic, and E. coli was added to the cells at a final concentration of 5.0×10⁷ E. coli cells/ml. L(+) Lactic acid was added together with E. coli at a final lactic acid concentration of 1.5 mM, 3 mM, or 6 mM. The cells were incubated at 37° C. for 4 hours and then harvested with QIAZOL reagent. RNA was extracted according to the QIAZOL protocol. The level of Matrix Metalloproteinase-9 (MMP-9) mRNA was measured by quantitative real-time PCR (qRT-PCR).

In FIG. 4, 2×10⁵ A549 cells were seeded in 500 μL growth medium per each well of a 24-well plate. Cells were treated with: (1) 2 uL PBS (control), (2) 5×10⁸ E. coli cells (E. coli), (3) or 5×10*8 cells E. coli with 0.25 ug, 0.5 ug, 1 ug, 2 ug, or 4 ug L-lactic acid. The cells were incubated for 4 hours at 37° C. Cells were harvested, RNA was extracted with an RNA extraction kit, the concentration of total RNA was measured, cDNA was obtained using a High-Capacity cDNA Reverse Transcription Kit, and then a TAQMAN real-time qPCR protocol was performed on the cDNA.

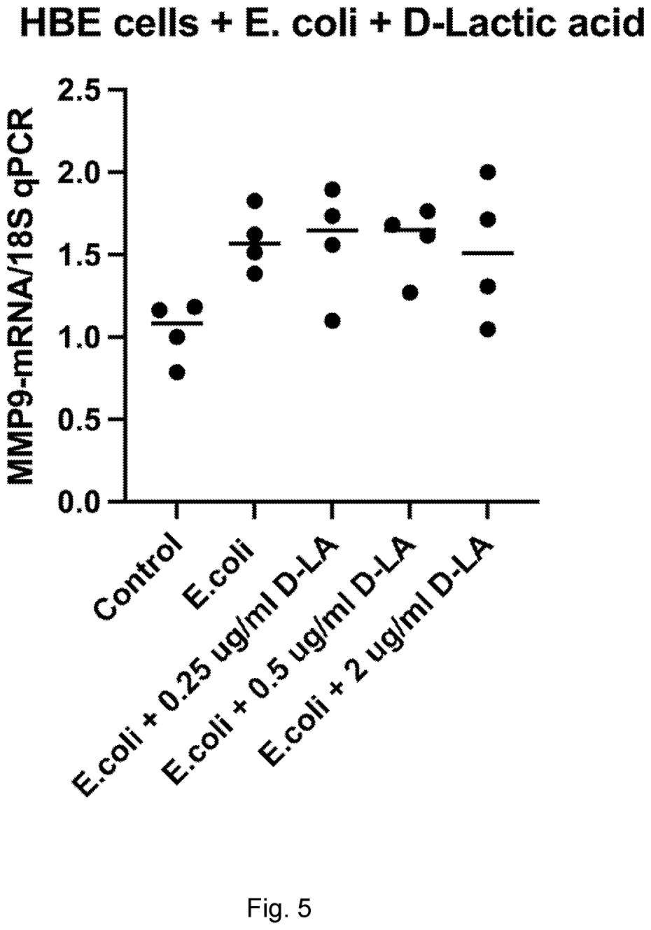

In FIG. 5, human bronchial epithelial (HBE) cells were seeded on a 6 well plate with 4×10⁵ cells in each well. 16 hours later, the medium was changed to fresh DULBECCO'S Modified Eagle Medium/Nutrient Mixture F-12 (DMEM/F12) without antibiotic, and E. coli was added to the cells at a final concentration of 5.0×10⁷ E. coli cells/ml. D(−) Lactic acid was added together with E. coli at a final lactic acid concentration of 0.25 ug/mL, 0.5 ug/mL, or 2 μg/mL. The cells were incubated at 37° C. for 4 hours and then harvested with QIAZOL reagent. RNA was extracted according to the QIAZOL protocol. The level of Matrix Metalloproteinase-9 (MMP-9) mRNA was measured by quantitative real-time PCR (qRT-PCR).

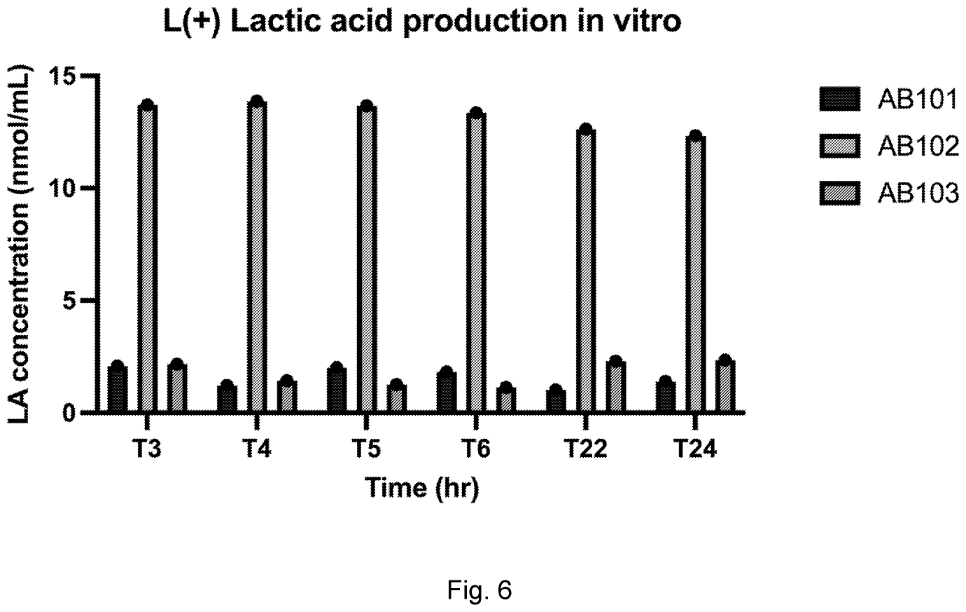

For FIG. 6, three bacterial strains AB101, AB102, and AB103 were assessed for their L(+) lactic acid output in vitro over the course of 24 hours. 5×10⁷ cells/mL of bacteria for each strain was cultured in MRS broth at 37 C in a shaker at 200 RPM for 16 hours. Cells were spun down and measured for L(+) lactic acid using the Lactate Colorimetric Assay kit (MAK064, SIGMA).

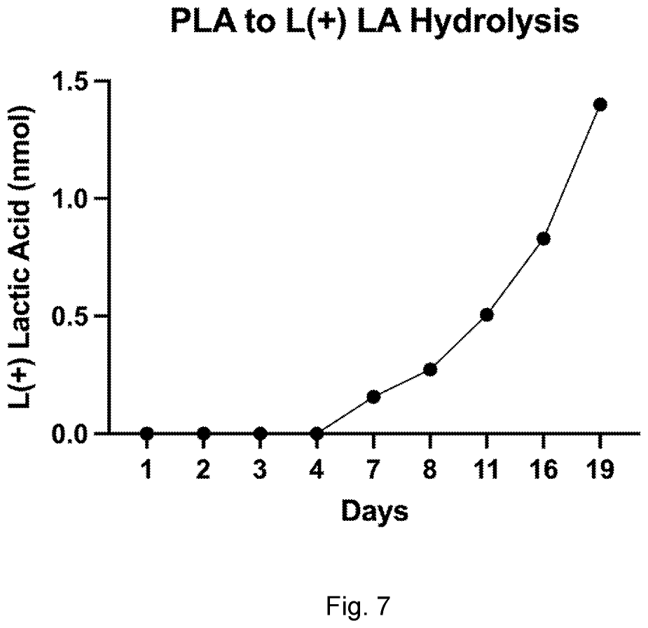

For FIG. 7, Poly-Lactic Acid powder (PLA) was dissolved in PBS at 37° C. and L(+) Lactic Acid was measured over time as the released product using the Lactate Colorimetric Assay kit (MAK064, SIGMA). A concentration of 3.3 mg/ml PLA was utilized by dissolving 0.1 g PLA powder in 30 ml PBS and incubating at 37° C. A sample volume of 200 μl from the PLA solution was taken out at each time point and measurement done according to the manufacturer's protocol.

What is claimed herein is:

1. A pharmaceutical composition comprising:
a) a lactic acid-producing compound selected from:
i) a polymeric compound that can produce lactic acid;
ii) a non-polymeric compound that can produce lactic acid; or
iii) lactic acid; and
b) a pharmaceutically acceptable excipient, stabilizer, or additive;
wherein the composition is formulated for administration to the lungs.

2. The pharmaceutical composition of claim 1, wherein the composition is formulated for administration by inhalation.

3. The pharmaceutical composition of claim 1, wherein the lactic acid-producing compound produces lactic acid upon delivery to a target tissue.

4. The pharmaceutical composition of claim 3, wherein the target tissue is a target bronchopulmonary tissue.

5. The pharmaceutical composition of claim 4, wherein the target bronchopulmonary tissue is the lungs, the trachea, the bronchi, the bronchioles, and/or the alveoli.

6. The pharmaceutical composition of claim 4, wherein the target tissue is a distal tissue site from the lungs delivered via the cardiovascular system or lymphatic system.

7. The pharmaceutical composition of claim 1, wherein the lactic acid-producing compound comprises a D enantiomer of lactic acid, an L enantiomer of lactic acid, or a racemic mixture of D and L enantiomers of lactic acid.

8. The pharmaceutical composition of claim 1, wherein the non-polymeric lactic acid-producing compound can be metabolized in the target tissue to produce lactic acid.

9. The pharmaceutical composition of claim 1, wherein the non-polymeric lactic acid-producing compound is an inorganic salt of lactic acid, an ester of lactic acid, or lactide.

10. The pharmaceutical composition of claim 9, wherein the inorganic salt of lactic acid is sodium lactate, potassium lactate, calcium lactate, or magnesium lactate.

11. The pharmaceutical composition of claim 9, wherein the ester of lactic acid is ethyl lactate, propyl lactate, butyl lactate, pentyl lactate, hexyl lactate, heptyl lactate, octyl lactate, nonyl lactate, decyl lactate, undecyl lactate, or dodecyl lactate.

12. The pharmaceutical composition of claim 1, wherein the polymeric lactic acid-producing compound can be hydrolyzed in the target tissue to produce lactic acid.

13. The pharmaceutical composition of claim 1, wherein the polymeric lactic acid-producing compound is a polylactic acid (PLA).

14. The pharmaceutical composition of claim 1, wherein the polylactic acid is poly(L-lactide) (PLLA), poly(D,L-lactide) (PDLLA), or poly(D-lactide) (PDLA).

15. The pharmaceutical composition of claim 1, wherein the polylactic acid is poly(D,L-lactide) (PDLLA).

16. The pharmaceutical composition of claim 1, wherein the polymeric lactic acid-producing compound is poly(lactic-co-glycolic acid) (PLGA).

17. The pharmaceutical composition of claim 1, wherein the polymeric lactic acid-producing compound is poly(lactic acid-co-caprolactone).

18. The pharmaceutical composition of claim 1, wherein the composition comprises at least 1.0% lactic acid-producing compound by weight.

19. The pharmaceutical composition of claim 1, wherein the composition comprises at least one excipient or at least one stabilizer.

20. The pharmaceutical composition of claim 1, further comprising at least one excipient.

21. The pharmaceutical composition of claim 1, further comprising at least two excipients.

22. The pharmaceutical composition of claim 20, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

23. The pharmaceutical composition of claim 20, wherein the excipient is leucine and/or trehalose.

24. The pharmaceutical composition of claim 1, wherein the composition comprises at least 5.0% excipient by weight.

25. The pharmaceutical composition of claim 1, wherein the composition comprises at least 5.0% of a first excipient by weight, and at least 5.0% of a second excipient by weight.

26. The pharmaceutical composition of claim 1, further comprising at least one stabilizer.

27. The pharmaceutical composition of claim 26, wherein the stabilizer comprises a surfactant.

28. The pharmaceutical composition of claim 26, wherein the stabilizer is selected from the group consisting of: mannitol, carboxymethyl cellulose (CMC), polyvinyl alcohol (PVA), polysorbate, and poloxamer.

29. The pharmaceutical composition of claim 26, wherein the stabilizer is a polysorbate; poloxamer; or polyvinyl alcohol.

30. The pharmaceutical composition of claim 26, wherein the stabilizer is Polysorbate 20, Polysorbate 40, Polysorbate 60, or Polysorbate 80.

31. The pharmaceutical composition of claim 26, wherein the stabilizer is Polysorbate 80.

32. The pharmaceutical composition of claim 26, wherein the stabilizer is Poloxamer 184, Poloxamer 185, Poloxamer 188, Poloxamer 234, Poloxamer 235, Poloxamer 238, Poloxamer 333, Poloxamer 334, Poloxamer 335, Poloxamer 338, Poloxamer 403, or Poloxamer 407.

33. The pharmaceutical composition of claim 26, wherein the stabilizer is poloxamer 188.

34. The pharmaceutical composition of claim 1, wherein the composition comprises at least 0.10% stabilizer by weight.

35. The pharmaceutical composition of claim 1, wherein the composition comprises at least one excipient and at least one stabilizer.

36. The pharmaceutical composition of claim 1, further comprising at least one of the following:
a) a pore-forming agent;
b) an adhesion agent;
c) a pH-modulating agent; and/or
d) an ester-hydrolysis-inducing agent.

37. The pharmaceutical composition of claim 36, wherein the pore-forming agent is selected from the group consisting of: NaCl, sucrose, polyethylene glycol (PEG), and polyvinylpyrrolidone (PVP).

38. The pharmaceutical composition of claim 36, wherein the adhesion agent is selected from the group consisting of: sugars, adhesive polymers, and amine-containing compounds.

39. The pharmaceutical composition of claim 36, wherein the pH-modulating agent is a buffer, an acid or a base.

40. The pharmaceutical composition of claim 36, wherein the ester-hydrolysis-inducing agent comprises an amine.

41. The pharmaceutical composition of claim 1, further comprising at least one acid-generating molecule that can generate acid, wherein the at least one acid-generating molecule is not lactic acid or does not comprise lactic acid.

42. The pharmaceutical composition of claim 41, wherein the acid-generating molecule is selected from the group consisting of: acetic acid, a hydroxy acid, a multiple functional acid, and an aromatic acid, and esters, salts, and polymers thereof.

43. The pharmaceutical composition of claim 41, wherein the acid-generating molecule is selected from the group consisting of: acetic acid, glycolic acid, citric acid, and salicylic acid, and esters, salts, and polymers thereof.

44. The pharmaceutical composition of claim 41, wherein the acid-generating molecule is acetylsalicylic acid.

45. The pharmaceutical composition of claim 1, wherein the composition comprises at least one additional therapeutic for a chronic bronchopulmonary disorder.

46. The pharmaceutical composition of claim 45, wherein the at least one additional therapeutic is microencapsulated.

47. The pharmaceutical composition of claim 45, wherein the at least one additional therapeutic is covalently linked with a degradable linker to the lactic acid-producing compound.

48. The pharmaceutical composition of claim 1, co-administered with at least one additional therapeutic for a chronic or infectious bronchopulmonary disorder.

49. The pharmaceutical composition of claim 48, wherein the at least one additional therapeutic is an anti-inflammatory, an antimicrobial, an antiviral, an antifungal, a vasodilator, or a bronchodilator.

50. The pharmaceutical composition of claim 49, wherein the anti-inflammatory is selected from the group consisting of: non-steroidal anti-inflammatory drugs (NSAIDs); corticosteroids; glucocorticoids; methotrexate; sulfasalazine; leflunomide; anti-tumor necrosis factor (TNF) medications; cyclophosphamide; pro-resolving lipid mediators; mycophenolate; opiates; and barbiturates.

51. The pharmaceutical composition of claim 49, wherein the antimicrobial is selected from the group consisting of: aminoglycosides; ansamycins; beta-lactams; bis-biguanides; carbacephems; carbapenems; cationic polypeptides; cephalosporins; fluoroquinolones; glycopeptides; iron-sequestering glycoproteins; linosamides; lipopeptides; macrolides; monobactams; nitrofurans; oxazolidinones; penicillins; polypeptides; quaternary ammonium compounds; quinolones; silver compounds; sulfonamides; and tetracyclines.

52. The pharmaceutical composition of claim 49, wherein the vasodilator is selected from the group consisting of: an angiotensin converting enzyme (ACE) inhibitor; an angiotensin receptor blocker (ARB); a calcium channel blocker (CCB); and a nitric-oxide-producing compound.

53. The pharmaceutical composition of claim 49, wherein the bronchodilator is selected from the group consisting of: albuterol, levalbuterol, epinephrine, salmeterol, formoterol, ipratropium bromide, tiotropium bromide, theophylline, and aminophylline.

54. The pharmaceutical composition of claim 1, wherein the composition is formulated as a bolus dose.

55. The pharmaceutical composition of claim 1, comprising:
a) the polymeric lactic acid-producing compound and/or the non-polymeric lactic acid-producing compound; and
b) a bolus dose of lactic acid.

56. The pharmaceutical composition of claim 1, wherein the composition is formulated as microspheres.

57. The pharmaceutical composition of claim 56, wherein the microspheres have a diameter of at least 1 μm to at most 1 mm.

58. The pharmaceutical composition of claim 1, wherein the composition comprises a plurality of dried particles.

59. The pharmaceutical composition of claim 58, wherein the dried particles have a Dv50 of at least 0.5 μm.

60. The pharmaceutical composition of claim 58, wherein the dried particles have a median mass aerodynamic diameter (MMAD) of at least 1.5 μm to at most 7.5 μm.

61. The pharmaceutical composition of claim 58, wherein the dried particles have a median mass aerodynamic diameter (MMAD) of at most 5.0 μm.

62. The pharmaceutical composition of claim 58, wherein the dried particles have a dispersibility of less than 2.0.

63. The pharmaceutical composition of claim 58, wherein the dried particles have a dispersibility of at least 0.5 to 1.0.

64. The pharmaceutical composition of claim 58, wherein the dried particles have a dispersibility of at least 0.9.

65. The pharmaceutical composition of claim 58, wherein the dried particles have a delivered dose of at least 25.0% to at most 125% of the composition by mass to a target tissue.

66. The pharmaceutical composition of claim 58, wherein the dried particles have a delivered dose of at least 30% of the lactic acid-producing compound by mass to a target tissue.

67. The pharmaceutical composition of claim 58, wherein the dried particles have a delivered dose of at least 7.8 mg per unit dose of the lactic acid-producing compound by mass to a target tissue.

68. The pharmaceutical composition of claim 58, wherein the dried particles have a delivered dose of at most 50 mg per unit dose of the lactic acid-producing compound by mass to a target tissue.

69. The pharmaceutical composition of claim 58, wherein the dried particles have a bulk density of at least 0.1 g/cm³ to 0.8 g/cm³.

70. The pharmaceutical composition of claim 58, wherein the dried particles have a bulk density of at least 0.5 g/cm³.

71. The pharmaceutical composition of claim 58, wherein the dried particles have a tapped density of at least 0.2 g/cm³ to 1.0 g/cm³.

72. The pharmaceutical composition of claim 58, wherein the dried particles have a tapped density of at least 0.6 g/cm³.

73. The pharmaceutical composition of claim 58, wherein the dried particles have a moisture content of at least 1.0% to 7.0% water by weight.

74. The pharmaceutical composition of claim 58, wherein the dried particles have a moisture content of at least 2.3% water by weight.

75. The pharmaceutical composition of claim 1, wherein the composition is formulated for delivery to the trachea, the bronchi, the bronchioles, and/or the alveoli.

76. The pharmaceutical composition of claim 1, wherein the composition is formulated for delivery to the lungs.

77. The pharmaceutical composition of claim 1, wherein the composition is formulated as a capsule or a tablet.

78. The pharmaceutical composition of claim 1, wherein the composition comprises at least 7.8 mg of the lactic acid-producing compound per unit dose that is deliverable to a target tissue.

79. The pharmaceutical composition of claim 1, wherein the composition comprises at least 15 mg of the lactic acid-producing compound per unit dose.

80. The pharmaceutical composition of claim 79, wherein the composition is formulated for delivery by an inhaler.

81. The pharmaceutical composition of claim 80, wherein the composition is formulated for delivery by a dry powder inhaler (DPI).

82. The pharmaceutical composition of claim 80, wherein the composition is formulated for delivery by a metered dose inhaler (MDI).

83. The pharmaceutical composition of claim 80, wherein the composition is formulated for delivery by a soft mist inhaler (SMI).

84. The pharmaceutical composition of claim 1, in combination with an inhaler.

85. An inhalation device for bronchopulmonary delivery comprising:
a) an inhaler; and
b) a container containing the pharmaceutical composition of claim 1.

86. The device of claim 85, wherein the inhaler is a dry powder inhaler (DPI).

87. The device of claim 85, wherein the inhaler is a metered dose inhaler (MDI).

88. The device of claim 85, wherein the inhaler is a soft mist inhaler (SMI).

89. The device of any one of claim 85, wherein the inhaler comprises:
a) a mouthpiece comprising an opening; and
b) means for aerosolizing or dispersing the pharmaceutical composition in the container.

90. A method of preparing a spray-dried pharmaceutical composition comprising a lactic acid-producing compound, comprising:
a) preparing a liquid feedstock comprising the lactic acid-producing compound;
b) introducing droplets of the liquid feedstock through an atomization nozzle into a drying chamber;
c) exposing the liquid feedstock droplets to heated, pressurized gas in the drying chamber to create dried particles; and
d) isolating dried particles of a predetermined range of diameters in a cyclone chamber, wherein the isolated dried particles comprise the lactic acid-producing compound.

91. A method of preparing a spray-dried pharmaceutical composition comprising a lactic acid-producing compound, comprising:
a) obtaining a liquid feedstock comprising the lactic acid-producing compound;
b) introducing droplets of the liquid feedstock through an atomization nozzle into a drying chamber;
c) exposing the liquid feedstock droplets to heated, pressurized gas in the drying chamber to create dried particles; and
d) isolating dried particles of a predetermined range of diameters in a cyclone chamber, wherein the isolated dried particles comprise the lactic acid-producing compound.

92. The method of claim 90, wherein the step of preparing the liquid feedstock comprises dissolving a solid feedstock into an aqueous solution.

93. The method of claim 90, wherein the step of preparing the liquid feedstock comprises dissolving a solid feedstock into an organic solution.

94. The method of claim 90, wherein the step of preparing the liquid feedstock comprises:
a) dissolving a lactic acid-producing compound into an organic solution;
b) dissolving a solid feedstock into an aqueous solution; and
c) combining the resultant solutions from (a) and (b) to produce a liquid feedstock.

95. The method of claim 90, wherein the solid feedstock comprises:
a) at least 50% lactic acid-producing compound by weight;
b) at least 10% excipient by weight; and/or
c) at least 1% stabilizer by weight.

96. The method of claim 90, wherein the solid feedstock comprises:
a) at least 50% lactic acid-producing compound by weight;
b) at least 5% of a first excipient by weight;
c) at least 5% of a second excipient by weight; and/or
d) at least 1% stabilizer by weight.

97. The method of claim 90, wherein the solid feedstock comprises at least 20% to at most 80% lactic acid-producing compound by weight.

98. The method of claim 90, wherein the solid feedstock comprises at least 1% to at most 15% excipient by weight.

99. The method of claim 90, wherein the solid feedstock comprises at least 2.5%-7.5% of a first excipient by weight, and at least 2.5%-7.5% of a second excipient by weight.

100. The method of claim 90, wherein the solid feedstock comprises at least 10% to at most 50% stabilizer by weight.

101. The method of claim 90, wherein the liquid feedstock comprises at least 0.1 g/L solid feedstock dissolved in an aqueous solution.

102. The method of claim 90, wherein the liquid feedstock comprises at least 5 g/L solid feedstock dissolved in an aqueous solution.

103. The method of claim 90, wherein the liquid feedstock comprises at least 0.01% to at most 10% solid feedstock dissolved in an aqueous solution.

104. The method of claim 90, wherein the liquid feedstock comprises at least 0.5% solid feedstock dissolved in an aqueous solution.

105. The method of claim 90, wherein 0.5 L of the liquid feedstock comprises:
a) at least 2.00 g lactic acid-producing compound;
b) at least 0.80 g excipient;
c) at least 1.2 g stabilizer;
d) at least 31.36 g organic solution; and/or
e) at least 464.64 g aqueous solution.

106. The method of claim 90, wherein 0.5 L of the liquid feedstock comprises:
a) at least 2.00 g lactic acid-producing compound;
b) at least 0.40 g of a first excipient;
c) at least 0.40 g of a second excipient;
d) at least 1.2 g stabilizer;
e) at least 31.36 g organic solution; and/or
f) at least 464.64 g aqueous solution.

107. The method of claim 90, wherein 0.5 L of the liquid feedstock comprises at least 0.1 g to at most 10 g lactic acid-producing compound.

108. The method of claim 90, wherein 0.5 L of the liquid feedstock comprises at least 0.1 g to at most 10 g excipient.

109. The method of any one of claim 90, wherein 0.5 L of the liquid feedstock comprises at least 0.05 g to at most 5 g of a first excipient, and at least 0.05 g to at most 5 g of a second excipient.

110. The method of claim 90, wherein 0.5 L of the liquid feedstock comprises at least 0.1 g to at most 10 g stabilizer.

111. The method of claim 90, wherein 0.5 L of the liquid feedstock comprises at least 10 g to at most 50 g organic solution.

112. The method of claim 90, wherein 0.5 L of the liquid feedstock comprises at least 420 g to at most 490 g aqueous solution.

113. The method of claim 90, wherein the liquid feedstock comprises:
a) at least 0.40% lactic acid-producing compound;
b) at least 0.16% excipient;
c) at least 0.24% stabilizer;
d) at least 6.27% organic solution; and/or
e) at least 92.93% aqueous solution.

114. The method of claim 90, wherein the liquid feedstock comprises:
a) at least 0.40% lactic acid-producing compound by weight;
b) at least 0.08% of a first excipient by weight;
c) at least 0.08% of a second excipient by weight;
d) at least 0.24% stabilizer;
e) at least 6.27% organic solution; and/or
f) at least 92.93% aqueous solution by weight.

115. The method of claim 90, wherein the liquid feedstock comprises at least 0.01% to at most 1.0% lactic acid-producing compound by weight.

116. The method of claim 90, wherein the liquid feedstock comprises at least 0.01% to at most 10% excipient by weight.

117. The method of claim 90, wherein the liquid feedstock comprises at least 0.005% to at most 5% of a first excipient by weight, and at least 0.005% to at most 5% of a second excipient by weight.

118. The method of claim 90, wherein the liquid feedstock comprises at least 0.01% to at most 1.0% stabilizer by weight.

119. The method of claim 90, wherein the liquid feedstock comprises at least 1% to at most 5% organic solution by weight.

120. The method of claim 90, wherein the liquid feedstock comprises at least 90% to at most 99.9% aqueous solution by weight.

121. The method of claim 90, wherein the lactic acid-producing compound is selected from:
a) a polymeric compound that can produce lactic acid;
b) a non-polymeric compound that can produce lactic acid; or
c) lactic acid.

122. The method of claim 90, wherein the non-polymeric lactic acid-producing compound is an inorganic salt of lactic acid, an ester of lactic acid, or lactide.

123. The method of claim 90, wherein the polymeric lactic acid-producing compound is a polylactic acid (PLA).

124. The method of claim 90, wherein the polylactic acid is poly(D,L-lactide) (PDLLA).

125. The method of claim 90, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcarnitine; propionylcarnitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

126. The method of claim 90, wherein the excipient is leucine and/or trehalose.

127. The method of claim 90, wherein the stabilizer is a polysorbate; poloxamer; or polyvinyl alcohol.

128. The method of claim 90, wherein the stabilizer is Poloxamer 188.

129. The method of claim 90, wherein the organic solution is acetone.

130. The method of claim 90, wherein the aqueous solution is water.

131. The method of claim 90, wherein the liquid feedstock further comprises at least one additional therapeutic.

132. The method of claim 131, wherein the at least one additional therapeutic is selected from the group consisting of: an anti-inflammatory, an antimicrobial, an antiviral, an antifungal, a vasodilator, and a bronchodilator.

133. The method of claim 90, wherein the atomization nozzle into the drying chamber has a diameter of at least 1.2 mm.

134. The method of claim 90, wherein the droplets of liquid feedstock produced by the atomization nozzle into the drying chamber have a diameter of at least 1.2 um.

135. The method of claim 90, wherein the droplets of liquid feedstock have a flow rate through the drying chamber of at least 5 g/min.

136. The method of claim 90, wherein the droplets of liquid feedstock have a flow rate through the drying chamber of at least 15 g/min.

137. The method of claim 90, wherein the droplets of liquid feedstock have a flow rate through the drying chamber of at most 1000 g/min.

138. The method of claim 90, wherein the heated, pressurized gas is heated before being inlet into the drying chamber.

139. The method of claim 90, wherein the heated, pressurized gas is inlet into the drying chamber at a temperature of at least 100° C.

140. The method of claim 90, wherein the heated, pressurized gas is inlet into the drying chamber at a temperature of at least 135° C.

141. The method of claim 90, wherein the heated, pressurized gas is inlet into the drying chamber at a temperature of at most 195° C.

142. The method of claim 90, wherein the heated, pressurized gas is outlet from the drying chamber at a temperature of at least 40° C.

143. The method of claim 90, wherein the heated, pressurized gas is outlet from the drying chamber at a temperature of at least 60° C.

144. The method of claim 90, wherein the heated, pressurized gas is outlet from the drying chamber at a temperature of at most 85° C.

145. The method of claim 90, wherein the heated, pressurized gas is pressurized before being inlet into the drying chamber.

146. The method of claim 90, wherein the heated, pressurized gas in the drying chamber has an atomization gas pressure of at least 10 pounds per square inch gauge (psig).

147. The method of claim 90, wherein the heated, pressurized gas in the drying chamber has an atomization gas pressure of at least 20 pounds per square inch gauge (psig).

148. The method of claim 90, wherein the heated, pressurized gas in the drying chamber has an atomization gas pressure of at most 150 pounds per square inch gauge (psig).

149. The method of claim 90, wherein the heated, pressurized gas has a flow rate through the drying chamber of at least 5 kg/hr.

150. The method of claim 90, wherein the heated, pressurized gas has a flow rate through the drying chamber of at least 18 kg/hr.

151. The method of claim 90, wherein the heated, pressurized gas has a flow rate through the drying chamber of at most 150 kg/hr.

152. The method of claim 90, wherein the heated, pressurized gas is outlet through the cyclone chamber.

153. The method of claim 90, wherein the step of exposing the liquid feedstock droplets to heated, pressurized gas in the drying chamber takes at most 8 hours.

154. The method of claim 90, wherein the dried particles isolated in the cyclone chamber have a median mass aerodynamic diameter (MMAD) of at least 1.5 μm to at most 7.5 μm.

155. The method of claim 90, wherein the dried particles isolated in the cyclone chamber have a median mass aerodynamic diameter (MMAD) of at least 4.0 μm.

156. The method of claim 90, wherein the dried particles isolated in the cyclone chamber have a median mass aerodynamic diameter (MMAD) of at most 5.0 μm.

157. The method of claim 90, wherein the step of isolating dried particles of a predetermined range of diameters in the cyclone chamber occurs continuously.

158. A method of delivering a spray-dried pharmaceutical composition comprising a lactic acid-producing compound to a subject, comprising:
a) obtaining an inhalation device for bronchopulmonary delivery comprising:
i) an inhaler; and
ii) a container containing a spray-dried pharmaceutical composition comprising a lactic acid-producing compound;
b) activating the inhaler to cause aerosolization or dispersal of the spray-dried pharmaceutical composition; and
d) inhaling the aerosolized or dispersed spray-dried pharmaceutical composition.

159. A method of delivering a spray-dried pharmaceutical composition comprising a lactic acid-producing compound to a subject, comprising:
a) obtaining an inhalation device for bronchopulmonary delivery comprising:
i) an inhaler; and
ii) a container containing the pharmaceutical composition of claim 1, wherein the pharmaceutical composition is spray-dried;
d) activating the inhaler to cause aerosolization or dispersal of the spray-dried pharmaceutical composition; and
e) inhaling the aerosolized or dispersed spray-dried pharmaceutical composition.

160. A method of delivering a spray-dried pharmaceutical composition comprising a lactic acid-producing compound to a subject, comprising:
a) obtaining the inhalation device of claim 85;
c) activating the inhaler to cause aerosolization or dispersal of the spray-dried pharmaceutical composition; and
d) inhaling the aerosolized or dispersed spray-dried pharmaceutical composition.

161. The method of claim 158, wherein the inhaler is a dry powder inhaler (DPI).

162. The method of claim 158, wherein the inhaler is a metered dose inhaler (MDI).

163. The method of claim 158, wherein the inhaler is a soft mist inhaler (SMI).

164. The method of claim 158, wherein the inhaler comprises:
a) a mouthpiece comprising an opening; and
b) means for aerosolizing or dispersing the spray-dried pharmaceutical composition in the container.

165. The method of claim 158, wherein the inhaler has an inspiration flow rate of at least 15 L/min to at most 60 L/min.

166. The method of claim 158, wherein at least 25% to at most 125% of the spray-dried pharmaceutical composition by mass is delivered to a target bronchopulmonary tissue.

167. The method of claim 158, wherein at least 30% of the spray-dried pharmaceutical composition by mass is delivered to a target bronchopulmonary tissue.

168. The method of claim 158, wherein the target bronchopulmonary tissue is the lungs, the trachea, the bronchi, the bronchioles, and/or the alveoli.

169. The method of claim 158, wherein the spray-dried pharmaceutical composition is delivered from the bronchopulmonary tissue to a distal tissue site via the cardiovascular system or lymphatic system.

170. A method of treating a subject in need thereof comprising administering through inhalation an effective dose of a pharmaceutical composition comprising a lactic acid-producing compound.

171. A method of treating a subject in need thereof comprising administering through inhalation an effective dose of the pharmaceutical composition of claim 1.

172. The method of claim 170, wherein the lactic acid-producing compound reduces neutrophilic inflammation in a target tissue.

173. The method of claim 172, wherein the target tissue is a target bronchopulmonary tissue.

174. The method of claim 173, wherein the target bronchopulmonary tissue is the lungs, the trachea, the bronchi, the bronchioles, and/or the alveoli.

175. The method of claim 172, wherein the target tissue is a distal tissue site from the lungs delivered via the cardiovascular system or lymphatic system.

176. The method of claim 170, wherein the subject has been diagnosed with or is at risk of developing a chronic or infectious bronchopulmonary disease.

177. The method of claim 176, wherein the chronic bronchopulmonary disease is selected from the group consisting of: asthma, bronchopulmonary dysplasia (BPD), chronic obstructive pulmonary disease (COPD), bronchiectasis, non-cystic fibrosis (CF) bronchiectasis, cystic fibrosis (CF), acute respiratory distress syndrome (ARDS), idiopathic pulmonary fibrosis (IPF), interstitial lung disease (LD), pleural effusion (PE), pulmonary hypertension (PAH), silicosis, and lung cancer.

178. The method of claim 170, wherein the lung cancer is small cell lung cancer (SCLC) or non-small cell lung cancer (NSCLC).

179. The method of claim 178, wherein the infectious bronchopulmonary disease is caused by or associated with an infectious agent selected from: adenovirus; coronavirus; influenza virus; parainfluenza virus; parvovirus; respiratory syncytial virus; rhinovirus; enterovirus; measles virus; rubella virus; varicella virus; Corynebacterium diphtheriae; Haemophilus influenzae; Legionella pneumophila; Bordetella pertussis; Mycobacterium tuberculosis; Streptococcus species; Pseudomonas species; Escherichia coli; Aspergillus species; Cryptococcus species; and Pneumocystis species.

180. The method of claim 170, wherein the pharmaceutical composition is a spray-dried pharmaceutical composition.

181. The method of claim 170, wherein the pharmaceutical composition is administered using the standard of care for the chronic or infectious bronchopulmonary disease.

182. The method of claim 170, wherein the pharmaceutical composition is administered using an inhaler.

183. The method of claim 182, wherein the inhaler is a dry powder inhaler (DPI).

184. The method of claim 182, wherein the inhaler is a metered dose inhaler (MDI).

185. The method of claim 182, wherein the inhaler is a soft mist inhaler (SMI).

186. The method of claim 170, wherein the effective dose of the pharmaceutical composition is at least 7.8 mg lactic acid-producing compound per unit dose.

187. The method of claim 170, wherein the pharmaceutical composition is co-administered with at least one additional therapeutic for a chronic or infectious bronchopulmonary disorder.

188. The method of claim 187, wherein the at least one additional therapeutic is an anti-inflammatory, an antimicrobial, an antiviral, an antifungal, a vasodilator, or a bronchodilator.

189. A unit dosage form comprising at least 1.0 mg to at most 100.0 mg of a pharmaceutical composition comprising a lactic acid-producing compound.

190. A unit dosage form comprising at least 1.0 mg to at most 100.0 mg of the pharmaceutical composition of claim 1.

191. A unit dosage form comprising at least 1.0 mg to at most 100.0 mg of the spray-dried pharmaceutical composition prepared by the methods of claim 90.

192. A unit dosage form comprising at least 15.0 mg to at most 100.0 mg of a pharmaceutical composition comprising at least 15 mg lactic acid-producing compound per unit dose.

193. The unit dosage form of claim 189, wherein the dosage is at least 1.0 mg pharmaceutical composition.

194. The unit dosage form of claim 189, wherein the dosage comprises at least 7.8 mg lactic acid-producing compound per unit dose that is deliverable to a target tissue.

195. The unit dosage form of claim 189, wherein the dosage comprises at least 15 mg lactic acid-producing compound per unit dose.

196. The unit dosage form of claim 189, wherein the pharmaceutical composition is a spray-dried pharmaceutical composition.

197. A pharmaceutical composition comprising:
a) a lactic acid-producing compound; and
b) a pharmaceutically acceptable excipient, stabilizer, or additive;
wherein the composition is formulated for oral administration.

198. The pharmaceutical composition of claim 197, wherein the lactic acid-producing compound is selected from:
a) a polymeric compound that can produce lactic acid;
b) a non-polymeric compound that can produce lactic acid; or
c) lactic acid.

199. The pharmaceutical composition of claim 197, wherein the non-polymeric lactic acid-producing compound is an inorganic salt of lactic acid, an ester of lactic acid, or lactide.

200. The pharmaceutical composition of claim 197, wherein the polymeric lactic acid-producing compound is a polylactic acid (PLA).

201. The pharmaceutical composition of claim 200, wherein the polylactic acid is poly(L-lactide) (PLLA), poly(D,L-lactide) (PDLLA), or poly(D-lactide) (PDLA).

202. The pharmaceutical composition of claim 197, wherein the composition comprises at least one excipient or at least one stabilizer.

203. The pharmaceutical composition of claim 202, wherein the excipient is selected from the group consisting of: De Man, Rogosa and Sharpe (MRS) growth medium; gelatin; whey isolate; sweet whey; reconstituted skim milk; maltodextrins; gluco-oligosaccharides; lacto-oligosaccharides; fructo-oligosaccharides; inulin; sodium caseinate; goat's milk; cow's milk; proline; carnitine; acetylcamitine; propionylcamitine; glutamate; glycine betaine; glycogen; trehalose; mannose; xylose; mannitol; sorbitol; maltose; dextrose; starch; lactose; sucrose; glucose; leucine; trileucine; sodium salts; potassium salts; lithium salts; and calcium salts.

204. The pharmaceutical composition of claim 202, wherein the excipient is leucine and/or trehalose.

205. The pharmaceutical composition of claim 202, wherein the stabilizer is a polysorbate; poloxamer; or polyvinyl alcohol.

206. The pharmaceutical composition of claim 202, wherein the stabilizer is Poloxamer 188.

207. The pharmaceutical composition of claim 202, wherein the additive is an adhesion agent.

208. The pharmaceutical composition of claim 197, wherein the composition comprises a plurality of dried particles.

209. The pharmaceutical composition of claim 197, wherein the composition comprises a plurality of spray-dried particles.

210. The pharmaceutical composition of claim 197, wherein the composition is formulated as a capsule or a tablet.

211. A method of treating a subject in need thereof comprising orally administering an effective dose of a pharmaceutical composition comprising a lactic acid-producing compound.

212. A method of treating a subject in need thereof comprising orally an effective dose of the pharmaceutical composition of claim 197.

213. The method of claim 211, wherein the lactic acid-producing compound reduces neutrophilic inflammation in a target tissue.

214. The method of claim 213, wherein the target tissue is a bronchopulmonary target tissue.

215. The method of claim 211, wherein the subject has been diagnosed with or is at risk of developing a chronic or infectious bronchopulmonary disorder.

216. The method of claim 211, wherein the pharmaceutical composition is a spray-dried pharmaceutical composition.

217. The method of claim 211, wherein the pharmaceutical composition is co-administered with at least one additional therapeutic.

218. The method of claim 217, wherein the at least one additional therapeutic is an anti-inflammatory, an antimicrobial, a vasodilator, or a bronchodilator.
